(19) United States
(12) Patent Application Publication
(10) Pub. No.: US 2008/0009552 A1
(43) Pub. Date: Jan. 10, 2008

(54) Markers of pre-term labor

(76) Inventors: Craig Pennell, Toronto (CA); Stephen Lye, Toronto (CA)

Correspondence Address: MCCARTHY TETRAULT LLP, BOX 48, SUITE 4700,, 66WELLINGTON STREET WEST, TORONTO, ON, M5K 1E6, CA

(21) Appl. No.: 11/727,179

(22) Filed: Mar. 23, 2007

Related U.S. Application Data
(60) Provisional application No. 60/785,147, filed on Mar. 23, 2006.

(51) Int. Cl. A61K 31/00; A61P 43/00; C12Q 1/68; G01N 33/48; G01N 33/53; G01N 33/68

(52) U.S. Cl. 514/789; 435/6; 436/501; 436/86; 436/94

(57) ABSTRACT

The invention relates to novel markers of pre-term labor, methods for assessing the status of pre-term labor using the markers, and methods for the diagnosis and therapy of pre-term labor.

### RELATED APPLICATIONS

This application claims priority from U.S. Provisional Patent Application No. 60/785,147, filed Mar. 23, 2006, entitled “Markers of Pre-Term Labour”. The entirety of which is herein incorporated by reference.

### FIELD OF INVENTIONS

The invention relates to novel markers of pre-term labor, methods for assessing pre-term labor using the markers, and methods for the detection, diagnosis, prediction, monitoring, preventing, and therapy of pre-term labor

### BACKGROUND OF THE INVENTION

Threatened pre-term labor occurs in many women during pregnancy and accounts for one third of all antenatal hospital admissions for pregnant women (15). Fortunately most women who present with threatened pre-term labor do not progress to pre-term delivery. Unfortunately the ability to predict the small percentage who will progress to delivery (true pre-term labor) within 7-10 days is poor (19-32), which leads to large numbers of women and their babies being hospitalized and unnecessarily exposed to potentially dangerous side effects associated with tocolytic and glucocorticoid administration. It is therefore of critical importance to develop new non-invasive methods to accurately and reliably diagnose pre-term labor which will 1) provide the means to effectively triage patients and so reduce demands on limited health care resources, and 2) limit the use of existing approaches such as antenatal glucocorticoids, short-term tocolysis, and transfer to tertiary perinatal facilities to those patients whose pre-term birth is imminent.

Currently there is no diagnostic test that will define those women in threatened pre-term labor (T-PTL) who will deliver within the next 7-10 days with both high positive and negative predictive values. A test based on fetal fibronectin (developed by Adeza, California) is widely used in the US and Australia: while it has a high negative predictive value, its positive predictive value is low (˜15%). A further major limitation of fetal fibronectin is that there are many contra-indications to performing this test limiting its use to only approximately 20% of women presenting with threatened pre-term labor.

### SUMMARY OF THE INVENTION

Applicants using micro-array technology, have identified distinct patterns of gene expression in women presenting with threatened pre-term labor who progress to delivery compared to those whose pregnancies continue to term. In particular, it was found that symptomatic women who present with threatened pre-term labor who progress to pre-term delivery (true pre-term labor) have different gene expression profiles in peripheral white blood cells when compared to those women who present with threatened pre-term labor who do not deliver within 48 hours. A test based on this gene expression “signature” will have both a high positive and negative predictive value for premature delivery in women presenting with signs and/or symptoms of pre-term labor. The use of these tests has significant advantages. They will result in a decrease in hospitalization, and administration of glucocorticoid and tocolytic therapy for symptomatic women that are not in true pre-term labor and thereby reduce costs to the health care system.

Thus, Applicants have developed a method for identifying markers associated with threatened pre-term labor that progresses to delivery. Using the method they analyzed samples from patients, and identified novel correlations between the expression of certain markers and threatened pre-term labor that progresses to delivery as well as markers associated with pregnancies that continue to term. The invention therefore provides a set of markers that can distinguish threatened pre-term labor that progresses to delivery. Methods are provided for use of these markers to distinguish between the patient groups, and to determine general courses of treatment.

In an aspect, the invention relates to a method of characterizing a biological sample by detecting or quantitating in the sample one or more polynucleotides extracted from the sample that are characteristic of pre-term labor or onset of pre-term labor the method comprising assaying for differential expression of polynucleotides in the sample. Differential expression of the polynucleotides can be determined by micro-array, hybridization or by amplification of the extracted polynucleotides.

The invention also relates to a method of characterizing or classifying a sample by detecting or quantitating in the sample one or more polypeptides extracted from the sample that are characteristic of pre-term labor or onset of pre-term labor, the method comprising assaying for differential expression of polypeptides in the sample. Differential expression of polypeptides can be assayed using procedures known in the art, including without limitation, separation techniques known in the art, antibody microarrays, or mass spectroscopy of polypeptides extracted from a sample.

An embodiment of the invention is directed to bioinformatic methods for analyzing gene expression data generated from nucleic acid micro-array experiments to identify further biomarker genes from various cell types. Another embodiment of the invention is directed to biomarker genes identified from mammalian (e.g., human, primate) peripheral blood cells at normal and/or abnormal states. The biomarker genes are useful as molecular targets for therapeutics of a disorder or disease in mammals.

The invention contemplates a gene expression “signature” identified using a method of the invention that is associated with delivery within about 48 hours in women presenting with idiopathic threatened pre-term labor. This signature provides a highly sensitive and specific test with both high positive and negative predictive values permitting diagnosis and prediction of birth.

The invention provides gene marker sets that distinguish preterm labor, term labor or onset of pre-term labor and uses therefor. A genetic marker set may comprise a plurality of genes comprising or consisting of at least 5, 10, 15, 20, 25, 30, 35, 40, 45, or 50 of the genes corresponding to the markers listed in Tables 1-6. In one aspect the genes correspond to the markers listed in: Table 1 for pre-term delivery less than 48 hours from clinical presentation; in Tables 2 or 5, or in one aspect Table 5, for pre-term delivery less than 14 days from clinical presentation; in Table 3 for pre-term delivery less than 34 weeks gestation; or in Table 4 or 6, or in one aspect Table 6, for pre-term delivery less than 37 weeks gestation. In certain aspects, the plurality of genes consists of at least 50, 60, 70, 80, 90, 100, 110, 120, 130, 140, 150, 160, 170, 180, 190, 200, 210, 220, 230, 240, 250, 260, 270, 280, 290, or 300 of the gene markers listed in Tables 1-6. In one aspect the gene markers correspond to the markers listed in: Table 1 for pre-term delivery less than 48 hours from clinical presentation; in Tables 2 or 5, or in one aspect Table 5, for pre-term delivery less than 14 days from clinical presentation; in Table 3 for pre-term delivery less than 34 weeks gestation; or in Table 4 or 6, or in one aspect Table 6, for pre-term delivery less than 37 weeks gestation. In an aspect, the gene marker sets comprise gene clusters which may be represented by dendograms or comprise genes in pathways of up and/or down regulated genes identified in accordance with the invention.

In embodiments of the invention, a gene is provided which is selected from the group consisting of the genes set forth in Tables 1-6, which gene is an up-regulated biomarker of pre-term labor. In one aspect the genes correspond to the those listed in: Table 1 for pre-term delivery less than 48 hours from clinical presentation; in Tables 2 or 5, or in one aspect Table 5, for pre-term delivery less than 14 days from clinical presentation; in Table 3 for pre-term delivery less than 34 weeks gestation; or in Table 4 or 6, or in one aspect Table 6, for pre-term delivery less than 37 weeks gestation, which gene is an up-regulated biomarker of pre-term labor.

In embodiments of the invention, a gene is provided which is selected from the group consisting of the genes set forth in Tables 1-6 which gene is a down-regulated biomarker of pre-term labor. In one aspect the genes correspond to the those listed in: Table 1 for pre-term delivery less than 48 hours from clinical presentation; in Tables 2 or 5, or in one aspect Table 5, for pre-term delivery less than 14 days from clinical presentation; in Table 3 for pre-term delivery less than 34 weeks gestation; or in Table 4 or 6, or in one aspect Table 6, for pre-term delivery less than 37 weeks gestation, which gene is a down-regulated biomarker of pre-term labor.

The invention also contemplates a sequence selected from the group consisting of the genes and sequences identified in Tables 1-6, and combinations thereof, which if a molecular target for therapeutics of pre-term labor or for the discovery of therapeutics for pre-term labor. In one aspect the genes and sequences correspond to those identified in: Table 1 for pre-term delivery less than 48 hours from clinical presentation; in Tables 2 or 5, or in one aspect Table 5, for pre-term delivery less than 14 days from clinical presentation; in Table 3 for pre-term delivery less than 34 weeks gestation; or in Table 4 or 6, or in one aspect Table 6, for pre-term delivery less than 37 weeks gestation, and combinations thereof, which if a molecular target for therapeutics of pre-term labor or for the discovery of therapeutics for pre-term labor.

The invention also contemplates protein marker sets that distinguish preterm labor and term labor, the protein marker sets comprising or consisting essentially of at least 5, 10, 15, 20, 25, 30, 35, 40, 45, or 50 of the proteins expressed by marker polynucleotides listed in Tables 1-6. In one aspect the proteins are those expressed by marker polynucleotides listed in: Table 1 for pre-term delivery less than 48 hours from clinical presentation; in Tables 2 or 5, or in one aspect Table 5, for pre-term delivery less than 14 days from clinical presentation; in Table 3 for pre-term delivery less than 34 weeks gestation; or in Table 4 or 6, or in one aspect Table 6, for pre-term delivery less than 37 weeks gestation. In certain aspects the plurality of proteins consists of at least 50, 60, 70, 80, 90, 100, 110, 120, 130, 140, 150, 160, 170, 180, 190, 200, 210, 220, 230, 240, 250, 260, 270, 280, 290, or 300 of the proteins expressed by marker polynucleotides listed in Tables 1-6. In one aspect the proteins are those expressed by marker polynucleotides listed in: Table 1 for pre-term delivery less than 48 hours from clinical presentation; in Tables 2 or 5, or in one aspect Table 5, for pre-term delivery less than 14 days from clinical presentation; in Table 3 for pre-term delivery less than 34 weeks gestation; or in Table 4 or 6, or in one aspect Table 6, for pre-term delivery less than 37 weeks gestation. In an aspect the protein marker sets comprise or consist of protein clusters, or proteins in pathways comprising the markers.

The protein markers of the invention disclosed herein including but not limited to native-sequence polypeptides, isoforms, chimeric polypeptides, all homologs, fragments, and precursors of the markers, including modified forms of the polypeptides and derivatives are referred to herein as “Pre-term Labor Marker(s)” or “PLM Markers”. Polynucleotides encoding Pre-term Labor Markers or expressing PLM Markers are referred to herein as “Pre-term Labor Polynucleotide Marker(s)”, “polynucleotides encoding Pre-term Labor Marker(s)” or “PLM Polynucleotides”. The PLM Markers and PLM Polynucleotides are sometimes collectively referred to herein as “marker(s)”.

PLM polynucleotides associated with pre-term labor or onset of pre-term labor identified in accordance with a method of the invention, (including the markers listed in Tables 1-6, or in one aspect markers listed in: Table 1 for pre-term delivery less than 48 hours from clinical presentation; Table 2 or 5, or in one aspect Table 5, for pre-term delivery less than 14 days for clinical presentation; in Table 3 for pre-term delivery less than 34 weeks gestation, or in Table 4 or 6, or in one aspect Table 6, for pre-term delivery less than 37 weeks gestation), and polypeptides expressed from the PLM polynucleotides, have application in the determination of the status of pre-term labor, and in particular in the detection of pre-term labor or onset of pre-term labor. Thus, the markers can be used for diagnosis, monitoring (i.e. monitoring progression or therapeutic treatment), prognosis, treatment, or classification of pre-term labor, or as markers before or after therapy.

The levels of PLM polynucleotides or PLM Markers in a sample may be determined by as described herein and generally known in the art. The expression levels may be determined by isolating and determining the level of nucleic acid transcribed from each PLM Polynucleotide. Alternatively or additionally, the levels of PLM Markers translated from mRNA transcribed from a PLM polynucleotide may be determined.

In accordance with methods of the invention, susceptibility to pre-term labor can be assessed or characterized, for example by detecting or identifying the presence in the sample of (a) a PLM Marker or fragment thereof; (b) a metabolite which is produced directly or indirectly by a PLM Marker; (c) a transcribed polynucleotide or fragment thereof having at least a portion with which a PLM Polynucleotide is substantially identical; and/or (c) a transcribed polynucleotide or fragment thereof, wherein the polynucleotide hybridizes with a PLM Polynucleotide.

In an aspect, the invention provides a method for characterizing or classifying a sample as pre-term labor comprising detecting a difference in the expression of a first plurality of genes relative to a control, the first plurality of genes consisting of at least 5, 10, 15, 20, 25, 30, 35, 40, 45, or 50 of the genes corresponding to the markers listed in Tables 1-6. In one aspect the genes correspond to the markers listed in: Table 1 for pre-term delivery less than 48 hours from clinical presentation; in Tables 2 or 5, or in one aspect Table 5, for pre-term delivery less than 14 days from clinical presentation; in Table 3 for pre-term delivery less than 34 weeks gestation; or in Table 4 or 6, or in one aspect Table 6, for pre-term delivery less than 37 weeks gestation. In particular aspects, the plurality of genes consists of at least 50, 60, 70, 80, 90, 100, 110, 120, 130, 140, 150, 160, 170, 180, 190, 200, 210, 220, 230, 240, 250, 260, 270, 280, 290, or 300 of the gene markers listed in Tables 1-6. In one aspect the gene markers are those listed in: In one aspect the proteins are those expressed by marker polynucleotides listed in: Table 1 for pre-term delivery less than 48 hours from clinical presentation; in Tables 2 or 5, or in one aspect Table 5, for pre-term delivery less than 14 days from clinical presentation; in Table 3 for pre-term delivery less than 34 weeks gestation; or in Table 4 or 6, or in one aspect Table 6, for pre-term delivery less than 37 weeks gestation. In another particular aspect, the control comprises polynucleotides derived from a pool of samples from individual term patients.

In an aspect, a method is provided characterizing susceptibility to pre-term labor by detecting PLM Markers or PLM Polynucleotides in a subject comprising:
(a) obtaining a sample from a subject;
(b) detecting or identifying in the sample PLM Markers or PLM Polynucleotides; and
(c) comparing the detected amount with an amount detected for a standard.

In an embodiment of the invention, a method is provided for detecting PLM Markers or PLM Polynucleotides in a subject or for diagnosing or monitoring in a subject a condition requiring regulation of labor comprising:

(a) obtaining a sample from a patient;

(b) detecting in the sample PLM Markers or PLM Polynucleotides; and

(c) comparing the detected amount with an amount detected for a standard.

The term “detect” or “detecting” includes assaying, imaging or otherwise establishing the presence or absence of the target markers or polynucleotides encoding the markers, subunits thereof, or combinations of reagent bound targets, and the like, or assaying for, imaging, ascertaining, establishing, or otherwise determining one or more factual characteristics of pre-term labor or similar conditions. The term encompasses diagnostic, prognostic, and monitoring applications for the PLM Markers and PLM Polynucleotides.

The invention also provides a method of assessing whether a patient has pre-term labor or a pre-disposition for pre-term labor comprising comparing:
(a) levels of PLM Markers or PLM Polynucleotides in a sample from the patient; and
(b) normal levels of PLM Markers or PLM Polynucleotides in samples of the same type obtained from control patients who delivered to term, wherein altered levels of the PLM Markers or PLM Polynucleotides relative to the corresponding normal levels of the markers or polynucleotides is an indication that the patient has pre-term labor or has a predisposition to pre-term labor.

In an embodiment of a method of the invention for assessing whether a patient has pre-term labor or a pre-disposition for pre-term labor, higher levels of PLM Markers or PLM Polynucleotides in a sample relative to the corresponding normal levels is an indication that the patient has pre-term labor or a pre-disposition for pre-term labor. In a particular embodiment the PLM Polynucleotides are the sequences listed on Tables 1-6, or in one aspect those listed in: Table 1 for pre-term delivery less than 48 hours from clinical presentation; in Tables 2 or 5, or in one aspect Table 5, for pre-term delivery less than 14 days from clinical presentation; in Table 3 for pre-term delivery less than 34 weeks gestation; or in Table 4 or 6, or in one aspect Table 6, for pre-term delivery less than 37 weeks gestation.

In another particular embodiment of a method of the invention for assessing whether a patient has pre-term labor or a pre-disposition for pre-term labor, lower levels of PLM Markers or PLM Polynucleotides in a sample relative to the corresponding normal levels is an indication that the patient has pre-term labor or a pre-disposition for pre-term labor. In a particular embodiment the PLM Polynucleotides are the sequences listed on Tables 1-6. In one aspect the sequences are those listed in: Table 1 for pre-term delivery less than 48 hours from clinical presentation; in Tables 2 or 5, or in one aspect Table 5, for pre-term delivery less than 14 days from clinical presentation; in Table 3 for pre-term delivery less than 34 weeks gestation; or in Table 4 or 6, or in one aspect Table 6, for pre-term delivery less than 37 weeks gestation.

In an embodiment of the invention, a method for screening or monitoring a subject for pre-term labor is provided comprising (a) obtaining a biological sample from a subject; (b) detecting the amount of PLM Markers or PLM Polynucleotides associated with pre-term labor in said sample; and (c) comparing said amount of PLM Markers or PLM Polynucleotides detected to a predetermined standard, where detection of a level of PLM Markers or PLM Polynucleotides that differs significantly from the standard indicates pre-term labor or onset of pre-term labor.

A significant difference between the levels of PLM Marker or PLM Polynucleotide levels in a patient and the normal levels is an indication that the patient has pre-term labor or a predisposition to pre-term labor.

In an embodiment the amount of PLM Marker(s) or PLM Polynucleotide(s) (e.g. see markers in Tables 1-6. In one aspect the markers are those listed in: Table 1 for pre-term delivery less than 48 hours from clinical presentation; in Tables 2 or 5, or in one aspect Table 5, for pre-term delivery less than 14 days from clinical presentation; in Table 3 for pre-term delivery less than 34 weeks gestation; or in Table 4 or 6, or in one aspect Table 6, for pre-term delivery less than 37 weeks gestation detected is greater than that of a standard and is indicative of pre-term labor. In another embodiment the amount of PLM Marker(s) or PLM Polynucleotide(s) (e.g. see markers in Tables 1-6 for the respective pre-term delivery groups) detected is lower than that of a standard and is indicative of pre-term labor or onset of pre-term labor.

A method of diagnosing or monitoring pre-term labor or onset of pre-term labor in a subject is provided comprising obtaining a biological sample from the subject, identifying polynucleotides in the sample associated with pre-term labor to identify pre-term labor of a particular etiology, and providing an individualized therapeutic strategy based on the etiology of pre-term labor identified.

In one aspect the invention provides a method for determining pre-term labor development potential in a patient at risk for the development of pre-term labor comprising the steps of determining the concentration of one or more markers in Tables 1-6 (or in one aspect, Table 1 for pre-term delivery less than 48 hours from clinical presentation; Table 2 or 5, or in one aspect Table 5, for pre-term delivery less than 14 days for clinical presentation; Table 3 for pre-term delivery less than 34 weeks gestation; or in Table 4 or 6, or in one aspect Table 6, for pre-term delivery less than 37 weeks gestation), in a sample (e.g. serum or plasma) from the patient, comparing the concentration of the markers to a cut-off concentration and determining pre-term development potential from the comparison, wherein concentrations of markers above the cut-off concentration are predictive of (e.g., correlate with) pre-term labor development in the patient.

In aspects of the methods of the invention, the methods are non-invasive for detecting pre-term labor, which in turn allow for diagnosis of a variety of conditions or diseases associated with such pre-term labor or conditions requiring regulation of labor.

In particular, the invention provides a non-invasive non-surgical method for detection, diagnosis, monitoring, or prediction of term or pre-term labor or onset of pre-term labor in a pregnant female comprising: obtaining a sample of blood, plasma, serum, urine or saliva or a tissue sample from the pregnant female; subjecting the sample to a procedure to detect PLM Marker(s) or PLM Polynucleotide(s) in the blood, plasma, serum, urine, saliva or tissue; detecting, diagnosing, and predicting term or pre-term labor by comparing the levels of PLM Marker(s) or PLM Polynucleotide(s) to the levels of PLM Marker(s) or PLM Polynucleotide(s) obtained from a pregnant non-laboring female.

In an embodiment, term or pre-term labor or onset of pre-term labor is detected, diagnosed, or predicted by determination of decreased levels of markers when compared to such levels obtained from the pregnant non-laboring female.

In another embodiment, term or pre-term labor or onset of pre-term labor is detected, diagnosed, or predicted by determination of increased levels of markers when compared to such levels obtained from the pregnant non-laboring female.

The invention provides a method for monitoring the progression of pre-term labor in a patient the method comprising:
(a) detecting PLM Markers or PLM Polynucleotides in a sample from the patient at a first time point;
(b) repeating step (a) at a subsequent point in time; and
(c) comparing the levels detected in (a) and (b), and therefrom monitoring the progression of the pre-term labor.

The invention also provides a method for assessing the potential efficacy of a test agent for preventing, inhibiting, or reducing pre-term labor or onset of pre-term labor, and a method of selecting an agent for inhibiting pre-term labor.

The invention also contemplates a method of assessing the potential of a test compound to contribute to pre-term labor or onset of pre-term labor comprising:
(a) maintaining separate aliquots of tissue from a patient in the presence and absence of the test compound; and
(b) comparing the levels of PLM Markers or PLM Polynucleotides in each of the aliquots.

A significant difference between the levels of PLM Markers or PLM Polynucleotides in an aliquot maintained in the presence of (or exposed to) the test compound relative to the aliquot maintained in the absence of the test compound, indicates that the test compound potentially contributes to pre-term labor or onset of pre-term labor.

A method for determining the effect of an environmental factor on pre-term birth comprising comparing polynucleotides associated with pre-term labor or onset of pre-term labor in the presence and absence of the environmental factor.

The invention further relates to a method of assessing the efficacy of a therapy for preventing, inhibiting, or reducing pre-term labor or onset of pre-term labor in a patient. A method of the invention comprises comparing: (a) levels of PLM Markers or PLM Polynucleotides in a sample from the patient obtained from the patient prior to providing at least a portion of a therapy to the patient; and (b) levels of PLM Markers or PLM Polynucleotides in a second sample obtained from the patient following therapy.

A significant difference between the levels of PLM Markers or PLM Polynucleotides in the second sample relative to the first sample is an indication that the therapy is efficacious for inhibiting pre-term labor or onset of pre-term labor.

In an embodiment, the method is used to assess the efficacy of a therapy for inhibiting pre-term labor or onset of pre-term labor, where lower levels of PLM Markers or PLM Polynucleotides relative to the first sample, is an indication that the therapy is efficacious for inhibiting the disease.

In an embodiment, the method is used to assess the efficacy of a therapy for inhibiting pre-term labor or onset of pre-term labor, where higher levels of PLM Markers or PLM Polynucleotides relative to the first sample, is an indication that the therapy is efficacious for inhibiting pre-term labor or onset of pre-term labor.

The “therapy” may be any therapy for treating pre-term labor or onset of pre-term labor in particular, including but not limited to therapeutics, and procedures and interventions such as antenatal glucocorticoids and tocolysis. A method of the invention can be used to evaluate a patient before, during, and after therapy.

Certain methods of the invention employ one or more polynucleotides capable of hybridizing to one or more PLM Polynucleotides. Thus, methods for monitoring pre-term labor or onset of pre-term labor are contemplated comprising detecting PLM Polynucleotide markers associated with pre-term labor.

Thus, the present invention relates to a method for diagnosing and monitoring pre-term labor or onset of pre-term labor in a sample from a subject comprising isolating polynucleotides, in particular mRNA, from the sample; and detecting PLM Polynucleotides in the sample. The presence of different levels of PLM Polynucleotides in the sample compared to a standard or control may be indicative of pre-term labor, stage of pre-term labor, onset of pre-term labor, and/or a positive prognosis.

In an embodiment of the invention, PLM Polynucleotide positive samples (e.g. higher levels of the PLM Polynucleotides compared to a normal control) are a negative diagnostic indicator. Positive tissue can be indicative of pre-term labor, advanced pre-term labor, onset of pre-term labor, or a poor prognosis.

In another embodiment of the invention, PLM Polynucleotide negative samples (e.g. lower levels of the PLM Polynucleotides compared to a normal control) are a negative diagnostic indicator. Negative tissues can be indicative of pre-term labor, advanced pre-term labor, onset of pre-term labor, or poor prognosis.

The invention provides methods for determining the presence or absence of pre-term labor in a subject comprising detecting in the sample levels of polynucleotides that hybridize to one or more PLM Polynucleotides, comparing the levels with a predetermined standard or cut-off value, and therefrom determining the presence or absence of pre-term labor in the subject. In an embodiment, the invention provides methods for determining the presence or absence of pre-term labor in a subject comprising (a) contacting a sample obtained from the subject with oligonucleotides that hybridize to one or more PLM Polynucleotides; and (b) detecting in the sample a level of polynucleotides that hybridize to the PLM Polynucleotides relative to a predetermined cut-off value, and therefrom determining the presence or absence of pre-term labor in the subject.

Within certain embodiments, the amount of polynucleotides that are mRNA are detected via polymerase chain reaction using, for example, oligonucleotide primers that hybridize to one or more PLM Polynucleotides, or complements of such polynucleotides. Within other embodiments, the amount of mRNA is detected using a hybridization technique, employing oligonucleotide probes that hybridize to one or more PLM Polynucleotides, or complements thereof.

When using mRNA detection, the method may be carried out by combining isolated mRNA with reagents to convert to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of one or more PLM Polynucleotides in the sample. For mRNA the analyzing step may be accomplished using Northern Blot analysis to detect the presence of PLM Polynucleotides. The analysis step may be further accomplished by quantitatively detecting the presence of PLM Polynucleotides in the amplification product, and comparing the quantity of marker detected against a panel of expected values for the known presence or absence of the markers in normal tissue derived using similar primers.

The invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to one or more PLM Polynucleotides to produce amplification products; (d) analyzing the amplification products to detect an amount of mRNA encoding the PLM Markers; and (e) comparing the amount of mRNA to an amount detected against a panel of expected values for normal tissue derived using similar nucleic acid primers.

In particular aspects of the invention, the methods described herein utilize the PLM Polynucleotides placed on a micro-array so that the expression status of each of the markers is assessed simultaneously.

In an embodiment, the invention provides a pre-term labour micro-array comprising a defined set of genes whose expression is significantly altered by pre-term labour.

The invention further relates to the use of the micro-array as a prognostic tool to predict pre-term delivery. In an embodiment, the pre-term labour micro-array discriminates between pre-term labor resulting from different etiologies.

In an embodiment, the invention provides for oligonucleotide arrays comprising marker sets described herein. The microarrays provided by the present invention may comprise probes to markers able to distinguish pre-term labor. In particular, the invention provides oligonucleotide arrays comprising probes to a subset or subsets of at least 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, or 300 gene markers (e.g. PLM Polynucleotides) up to a full set of markers which distinguish pre-term labor patients or samples.

The level of expression of the PLM Polynucleotides may be assessed by determining the levels of specific proteins expressed from the polynucleotides (i.e. the levels of the PLM Markers). Certain methods of the invention employ binding agents (e.g. antibodies) that specifically recognize PLM Markers.

In an embodiment, the invention provides methods for determining the presence or absence of pre-term labor or onset of pre-term labor, in a patient, comprising the steps of (a) contacting a biological sample obtained from a patient with one or more binding agent that specifically binds to one or more PLM Markers associated with pre-term labor; and (b) detecting in the sample an amount of marker that binds to the binding agent, relative to a predetermined standard or cut-off value, and therefrom determining the presence or absence of pre-term labor in the patient.

In another embodiment, the invention relates to a method for diagnosing and monitoring pre-term labor in a subject by quantitating one or more PLM Markers associated with pre-term labor in a biological sample from the subject comprising (a) reacting the biological sample with one or more binding agent specific for the PLM Markers (e.g. an antibody) that are directly or indirectly labelled with a detectable substance; and (b) detecting the detectable substance.

In another aspect the invention provides a method for using an antibody to detect expression of one or more PLM Marker in a sample, the method comprising: (a) combining antibodies specific for one or more PLM Marker with a sample under conditions which allow the formation of antibody:marker complexes; and (b) detecting complex formation, wherein complex formation indicates expression of the marker in the sample. Expression may be compared with standards and is diagnostic of pre-term labor.

PLM Markers levels can be determined by constructing an antibody microarray in which binding sites comprise immobilized, preferably monoclonal, antibodies specific to a substantial fraction of marker-derived proteins of interest.

The invention also relates to kits for carrying out the methods of the invention. In an embodiment, the kit is for assessing whether a patient is afflicted with a pre-term labor and it comprises reagents for assessing one or more PLM Markers or PLM Polynucleotides. In another embodiment, the invention provides diagnostic tools, and kits for detecting, diagnosing, and predicting the presence or impending onset of premature or pre-term labor by monitoring levels of PLM Markers or a PLM Polynucleotides.

The invention further provides kits comprising the marker sets described herein. In an aspect the kit contains a micro-array ready for hybridization to target PLM Polynucleotides, plus software for the data analyses.

The invention also provides a diagnostic composition comprising a PLM Marker or a PLM Polynucleotide. A composition is also provided comprising a probe that specifically hybridizes to PLM Polynucleotides, or a fragment thereof, or an antibody specific for PLM Markers or a fragment thereof. In another aspect, a composition is provided comprising one or more PLM Polynucleotide specific primer pairs capable of amplifying the polynucleotides using polymerase chain reaction methodologies. The probes, primers or antibodies can be labeled with a detectable substance.

Still further the invention relates to therapeutic applications for pre-term labor employing PLM Markers and PLM Polynucleotides, and/or binding agents for the markers.

In an aspect, the invention relates to pharmaceutical compositions comprising PLM Markers or parts thereof associated with pre-term labor, or binding agents (e.g antibodies) specific for PLM Markers associated with pre-term labor, and a pharmaceutically acceptable carrier, excipient, or diluent.

The invention provides a method of treating or preventing pre-term labor or onset of pre-term labor in a subject afflicted with or at risk of developing pre-term labor comprising administering to the subject an effective amount of an agonist of a down-regulated PLM Marker or PLM Polynucleotide. The term agonist is used in its broadest sense. Agonist can include any agent that results in activation, enhancement or alteration of the presence of a down-regulated PLM Marker or PLM Polynucleotide.

The invention provides a method of treating or preventing pre-term labor or onset of pre-term labor in a subject having or at risk of developing pre-term labor comprising administering to the subject an effective amount of an antagonist of an up-regulated PLM Marker or PLM Polynucleotide. The term antagonist or antagonizing is used in its broadest sense. Antagonism can include any mechanism or treatment that results in inhibition, inactivation, blocking or reduction or alteration of the presence of an up-regulated PLM Marker or PLM Polynucleotide. Examples of antagonists are antibodies specific for PLM Markers, binding agents for PLM Markers, and inhibitors of PLM Polynucleotides (e.g. antisense).

A method for treating or preventing pre-term labor or onset of pre-term labor in a subject is provided comprising administering to a subject in need thereof antibodies specific for PLM Markers. In an aspect the invention provides a method of treating a subject afflicted with or at risk of developing pre-term labor comprising inhibiting expression of one or more PLM Marker or PLM Polynucleotide.

In another aspect, the invention provides antibodies specific for PLM Markers associated with pre-term labor that can be used to inhibit PLM Marker or PLM Polynucleotide expression.

The invention contemplates a method of using antagonists or agonists of PLM Markers or PLM Polynucleotides or parts thereof in the preparation or manufacture of a medicament for the prevention or treatment of pre-term labor.

In an aspect the invention contemplates a method of using PLM Markers or parts thereof, antibodies specific for PLM Markers, or inhibitor of PLM Polynucleotides (e.g. antisense) in the preparation or manufacture of a medicament for the prevention or treatment of pre-term labor or onset of pre-term labor.

The invention also provides a method for stimulating or enhancing in a subject production of antibodies directed against one or more up-regulated PLM Marker. The method comprises administering to the subject one or more up-regulated PLM Marker, peptides derived therefrom, or chemically produced (synthetic) peptides, or any combination of these molecules of the invention in a dose effective for stimulating or enhancing production of the antibodies.

The invention contemplates the methods, compositions, and kits described herein using additional markers associated with pre-term labor (e.g. fibronectin). The methods described herein may be modified by including reagents to detect the additional markers, or polynucleotides for the markers.

In embodiments of the invention the methods, compositions and kits use one or more of the markers listed in Tables 1-6. In one aspect the markers correspond to the markers listed in: Table 1 for preterm delivery less than 48 hours from clinical presentation; Table 2 or 5, or in one aspect Table 5, for pre-term delivery less than 14 days for clinical presentation; Table 3 for pre-term delivery less than 34 weeks gestation, or Table 4 or 6, or in one aspect Table 6, for pre-term delivery less than 37 weeks gestation.

In another embodiment, they use a panel of markers selected from the markers listed in Tables 1-6 for the respective pre-term delivery groups, in particular a panel comprising two or more of the markers in Tables 1-6 for the respective pre-term delivery groups.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1 illustrates the sample processing method used in the examples.

FIG. 2 presents the baseline clinical data of the 40 women recruited into this study split based on whether they delivered within the 14 days after their clinical presentation.

FIG. 3 a scatter plot of all 19200 EST on the microarray comparing gene expression in leukocytes between the group of women who progressed to delivery within 14 days and those of delivered greater than 14 days after presentation.

FIG. 4 illustrates a Non-hierarchical Cluster Analysis: Prediction of Timing of Delivery.

FIG. 5 illustrates the four specific outcome groups evaluated in this study.

FIG. 6 is a schematic illustrating the determination of Class Prediction: Cross Validation and Permutation p-value.

FIG. 7 is a table illustrating the Cross Validation and Permutation p-value.

### DETAILED DESCRIPTION OF THE INVENTION

Methods are provided for detecting the presence of pre-term labor in a sample, the absence of pre-term labor, stage of pre-term labor, and other characteristics of pre-term labor that are relevant to prevention, diagnosis, monitoring, characterization, and therapy of pre-term labor in a patient. Methods are also provided for assessing the efficacy of one or more test agents for preventing, inhibiting, or reducing pre-term labor, assessing the efficacy of a therapy for pre-term labor, monitoring the progression of pre-term labor, selecting an agent or therapy for pre-term labor, treating a patient afflicted with pre-term labor, preventing, inhibiting, or reducing pre-term labor in a patient, and assessing the potential of a test compound to cause pre-term labor. In one embodiment, the invention provides a method of using gene expression profiles from peripheral white blood cells or decidual cells of symptomatic women in threatened pre-term labor to predict pre-term birth.

Glossary

“Pre-term labor”, refers to the premature onset of labor resulting in expulsion from the uterus of a viable infant before the normal end of gestation (i.e. pre-term birth or delivery), or more particularly, onset of labor with effacement and dilation of the cervix before the 37th week of gestation. It may or may not be associated with vaginal bleeding or rupture of membranes. Pre-term labor may be related to factors including without limitation infection (eg, bacterial vaginosis [BV], sexually transmitted diseases [STDs], urinary tract infections, chorioamnionitis), uterine distention (eg, multiple gestation, polyhydramnios), uterine distortion (eg, müllerian duct abnormalities, fibroid uterus), compromised structural support of the cervix (eg, incompetent cervix, previous cone biopsy or loop electrosurgical excision procedure [LEEP]), abruptio placentae, uteroplacental insufficiency (eg, hypertension, insulin-dependent diabetes, drug abuse, smoking, alcohol consumption), stress either indirectly by associated risk behaviors or by direct mechanisms including fetal stress.

“Threatened pre-term labor” refers to premature onset of labor before the 37th week of gestation followed by a continuation of pregnancy to term (“term labor”) or pre-term labor. Symptoms of threatened pre-term labor include without limitation regular uterine contractions, cervical dilation 0-4 cm, and/or intact fetal membranes.

“Micro-array” and “array,” refer to nucleic acid or nucleotide arrays or protein or peptide arrays that can be used to detect biomolecules associated with pre-term labor, for instance to measure gene expression. A variety of arrays are made in research and manufacturing facilities worldwide, some of which are available commercially. By way of example, spotted arrays and in situ synthesized arrays are two kinds of nucleic acid arrays that differ in the manner in which the nucleic acid materials are placed onto the array substrate. A widely used in situ synthesized oligonucleotide array is GeneChip™ made by Affymetrix, Inc. Oligonucleotide probes that are 20- or 25-base long can be synthesized in silico on the array substrate. These arrays can achieve high densities (e.g., more than 40,000 genes per cm²). Generally spotted arrays have lower densities, but the probes, typically partial cDNA molecules, are much longer than 20- or 25-mers. Examples of spotted cDNA arrays include LifeArray made by Incyte Genomics and DermArray made by IntegriDerm (or Invitrogen). Pre-synthesized and amplified cDNA sequences are attached to the substrate of spotted arrays. Protein and peptide arrays also are known [(see for example, Zhu et al., Science 293: 2101 (2001)].

The terms “sample”, “biological sample”, and the like mean a material known or suspected of expressing or containing one or more PLM Polynucleotides and/or one or more PLM Markers. A test sample can be used directly as obtained from the source or following a pretreatment to modify the character of the sample. A sample can be derived from any biological source, such as tissues, extracts, or cell cultures, including cells, cell lysates, and physiological fluids, such as, for example, whole blood, plasma, serum, saliva, ocular lens fluid, cerebral spinal fluid, sputum, sweat, urine, milk, ascites fluid, synovial fluid, peritoneal fluid, and the like.

The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like.

In embodiments of the invention the sample is blood, in particular blood cells, particularly maternal peripheral blood cells, more particularly mononuclear leukocytes. In one embodiment the sample is peripheral white blood cells. In another embodiment the sample is decidual cells.

The samples that may be analyzed in accordance with the invention include polynucleotides from clinically relevant sources, preferably expressed RNA or a nucleic acid derived therefrom (cDNA or amplified RNA derived from cDNA that incorporates an RNA polymerase promoter). The target polynucleotides can comprise RNA, including, without limitation total cellular RNA, poly(A)+messenger RNA (mRNA) or fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly(A)⁺ RNA are well known in the art, and are described generally, for example, in Sambrook et al., (1989, Molecular Cloning—A Laboratory Manual (2^{nd} Ed.), Vols. 1-3, Cold Spring Harbor Laboratory, Cold Spring Harbor, N.Y.) and Ausubel et al, eds. (1994, Current Protocols in Molecular Biology, vol. 2, Current Protocols Publishing, New York). RNA may be isolated from eukaryotic cells by procedures involving lysis of the cells and denaturation of the proteins contained in the cells. Additional steps may be utilized to remove DNA. Cell lysis may be achieved with a nonionic detergent, followed by microcentrifugation to remove the nuclei and hence the bulk of the cellular DNA. (See Chirgwin et al., 1979, Biochemistry 18:5294-5299). Poly(A)+ RNA can be selected using oligo-dT cellulose (see Sambrook et al., 1989, Molecular Cloning—A Laboratory Manual (2nd Ed.), Vols. 1-3, Cold Spring Harbor Laboratory, Cold Spring Harbor, N.Y.). In the alternative, RNA can be separated from DNA by organic extraction, for example, with hot phenol or phenol/chloroform/isoamyl alcohol.

It may be desirable to enrich mRNA with respect to other cellular RNAs, such as transfer RNA (tRNA) and ribosomal RNA (rRNA). Most mRNAs contain a poly(A) tail at their 3′ end allowing them to be enriched by affinity chromatography, for example, using oligo(dT) or poly(U) coupled to a solid support, such as cellulose or Sephadex™ (see Ausubel et al., eds., 1994, Current Protocols in Molecular Biology, vol. 2, Current Protocols Publishing, New York). Bound poly(A)+ mRNA is eluted from the affinity column using 2 mM EDTA/0.1% SDS.

A sample of RNA can comprise a plurality of different mRNA molecules each with a different nucleotide sequence. In an aspect of the invention, the mRNA molecules in the RNA sample comprise at least 100 different nucleotide sequences.

Target polynucleotides can be detectably labeled at one or more nucleotides using methods known in the art. The label is preferably uniformly incorporated along the length of the RNA, and more preferably, is carried out at a high degree of efficiency. The detectable label can be a luminescent label, fluorescent label, bio-luminescent label, chemi-luminescent label, radiolabel, and colorimetric label. In a particular embodiment, the label is a fluorescent label, such as a fluorescein, a phosphor, a rhodamine, or a polymethine dye derivative. Commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.).

Target polynucleotides from a patient sample can be labeled differentially from polynucleotides of a standard. The standard can comprise target polynucleotides from normal individuals (i.e., those not afflicted with or pre-disposed to pre-term labor), in particular pooled from samples from normal individuals. The target polynucleotides can be derived from the same individual, but taken at different time points, and thus indicate the efficacy of a treatment by a change in expression of the markers, or lack thereof, during and after the course of treatment.

The terms “subject”, “individual” or “patient” refer to a warm-blooded animal such as a mammal. In particular, the terms refer to a human. A subject, individual or patient may be afflicted with or suspected of having or being pre-disposed to pre-term labor. The present invention may be particularly useful for determining pre-term labor development potential in at-risk patients suffering from particular pre-term labor predisposing conditions. Pre-term labor predisposing conditions include without limitation a previous history of preterm delivery, previous history of a second-trimester abortion, uterine factors such as uterine volume increase, uterine anomalies, trauma and infection.

The term “PLM Marker” or “Pre-term labor Markers” includes a marker associated with pre-term labor. The term includes native-sequence polypeptides isoforms, chimeric polypeptides, complexes, all homologs, fragments, precursors, and modified forms and derivatives of the markers. The term includes a marker associated with pre-term labor identified using a method of the invention, in particular a marker expressed by a polynucleotide listed in Tables 1-6, or in one aspect a marker expressed by a polynucleotide listed in: Table 1 for pre-term delivery less than 48 hours from clinical presentation; Table 2 or 5, or in one aspect Table 5, for pre-term delivery less than 14 days for clinical presentation; Table 3 for pre-term delivery less than 34 weeks gestation, or in Table,4 or 6 or in one aspect Table 6, for pre-term delivery less than 37 weeks gestation.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 70-80%, preferably at least about 85%, more preferably at least about 90%, most preferably at least about 95% amino acid sequence identity with a native-sequence polypeptide. Particular polypeptide variants have at least 70-80%, 85%, 90%, 95% amino acid sequence identity to the sequences of the proteins expressed by the polynucleotides identified in Table 1. Such variants include, for instance, polypeptides wherein one or more amino acid residues are added to, or deleted from, the N- or C-terminus of the full-length or mature sequences of the polypeptide, including variants from other species, but excludes a native-sequence polypeptide.

The invention also includes polypeptides that are substantially identical to the sequences of a PLM Marker, in particular a pre-term labor marker, more particularly a marker expressed by a polynucleotide listed in Tables 1-6 (e.g. at least about 45%, preferably 50%, 55%, 60%, 65%, 70%, 75%, 80%, 85%, 90%,95%,97%,98%, or 99% sequence identity), and in particular polypeptides that retain the immunogenic activity of the corresponding native-sequence polypeptide. In one aspect the marker is expressed by a polynucleotide listed in: Table 1 for pre-term delivery less than 48 hours from clinical presentation; Table 2 or 5, or in one aspect Table 5, for pre-term delivery less than 14 days for clinical presentation; Table 3 for pre-term delivery less than 34 weeks gestation, or in Table,4 or 6 or in one aspect Table 6, for pre-term delivery less than 37 weeks gestation (e.g. at least about 45%, preferably 50%, 55%, 60%, 65%, 70%, 75%, 80%, 85%, 90%,95%, 97%, 98%, or 99% sequence identity), and in particular polypeptides that retain the immunogenic activity of the corresponding native-sequence polypeptide.

Percent identity of two amino acid sequences, or of two nucleic acid sequences is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410,1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

An allelic variant may also be created by introducing substitutions, additions, or deletions into a polynucleotide encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, H is), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, H is). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a polypeptide homolog, for example, a murine or rat polypeptide.

PLM Markers include chimeric or fusion proteins. A “chimeric protein” or “fusion protein” comprises all or part (preferably biologically active) of a PLM Marker operably linked to a heterologous polypeptide (i.e., a polypeptide other than a PLM Marker). Within the fusion protein, the term “operably linked” is intended to indicate that a PLM Marker and the heterologous polypeptide are fused in-frame to each other. The heterologous polypeptide can be fused to the N-terminus or C-terminus of a PLM Marker. A useful fusion protein is a GST fusion protein in which a PLM Marker is fused to the C-terminus of GST sequences. Another example of a fusion protein is an immunoglobulin fusion protein in which all or part of a PLM Marker is fused to sequences derived from a member of the immunoglobulin protein family. Chimeric and fusion proteins can be produced by standard recombinant DNA techniques.

A modified form of a polypeptide referenced herein includes modified forms of the polypeptides and derivatives of the polypeptides, including but not limited to glycosylated, phosphorylated, acetylated, methylated or lapidated forms of the polypeptides.

PLM Markers may be prepared by recombinant or synthetic methods, or isolated from a variety of sources, or by any combination of these and similar techniques.

“Pre-term Labor Polynucleotides”, “PLM Polynucleotide(s)”, or “polynucleotides encoding pre-term labor markers” refers to polynucleotides associated with pre-term labor and/or encoding PLM Markers including native-sequence polypeptides, polypeptide variants including a portion of a polypeptide, an isoform, precursor, complex, a chimeric polypeptide, or modified forms and derivatives of the polypeptides. A PLM Polynucleotides can be a polynucleotide listed in Table 1 or a fragment thereof.

PLM Polynucleotides include complementary nucleic acid sequences, and nucleic acids that are substantially identical to these sequences (e.g. at least about 45%, preferably 50%, 55%, 60%, 65%, 70%, 75%, 80%, 85%, 90%, 95%, 97%, 98%, or 99% sequence identity).

PLM Polynucleotides also include sequences that differ from a native sequence due to degeneracy in the genetic code. As one example, DNA sequence polymorphisms within the nucleotide sequence of a PLM Polynucleotide may result in silent mutations that do not affect the amino acid sequence. Variations in one or more nucleotides may exist among individuals within a population due to natural allelic variation. DNA sequence polymorphisms may also occur which lead to changes in the amino acid sequence of a polypeptide.

Polynucleotides also include nucleic acids that hybridize under stringent conditions, preferably high stringency conditions to a PLM Polynucleotide. Appropriate stringency conditions which promote DNA hybridization are known to those skilled in the art, or can be found in Current Protocols in Molecular Biology, John Wiley & Sons, N.Y. (1989), 6.3.1-6.3.6. For example, 6.0× sodium chloride/sodium citrate (SSC) at about 45° C., followed by a wash of 2.0×SSC at 50° C. may be employed. The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C.

PLM Polynucleotides also include truncated nucleic acids or nucleic acid fragments and variant forms of the nucleic acids that arise by alternative splicing of an mRNA corresponding to a DNA.

PLM Polynucleotide markers are intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The polynucleotides for use in the methods of the invention may be of any length suitable for a particular method. In certain applications the term refers to antisense polynucleotides (e.g. mRNA or DNA strand in the reverse orientation to sense polynucleotide markers).

“Statistically different levels”, “significantly altered”, or “significant difference” in levels of markers in a patient sample compared to a control or standard (e.g. normal levels or levels in other samples from a patient) may represent levels that are higher or lower than the standard error of the detection assay. In particular embodiments, the levels may be 1.5, 2, 3, 4, 5, or 6 times higher or lower than the control or standard.

“Binding agent” refers to a substance such as a polypeptide or antibody that specifically binds to one or more PLM Marker. A substance “specifically binds” to one or more PLM Marker if is reacts at a detectable level with one or more PLM Marker, and does not react detectably with peptides containing an unrelated or different sequence. Binding properties may be assessed using an ELISA, which may be readily performed by those skilled in the art (see for example, Newton et al, Develop. Dynamics 197: 1-13, 1993).

A binding agent may be a ribosome, with or without a peptide component, an aptamer, an RNA molecule, or a polypeptide. A binding agent may be a polypeptide that comprises one or more PLM Marker sequence, a peptide variant thereof, or a non-peptide mimetic of such a sequence.

An aptamer includes a DNA or RNA molecule that binds to nucleic acids and proteins. An aptamer that binds to a protein (or binding domain) or a PLM Polynucleotide can be produced using conventional techniques, without undue experimentation. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

Antibodies for use in the present invention include but are not limited to monoclonal or polyclonal antibodies, immunologically active fragments (e.g. a Fab or (Fab)₂ fragments), antibody heavy chains, humanized antibodies, antibody light chains, genetically engineered single chain Fᵥ molecules (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native or recombinant PLM Markers may be utilized to prepare antibodies. See, for example, Kohler et al. (1975) Nature 256:495-497; Kozbor et al. (1985) J. Immunol. Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. Antibodies specific for a PLM Marker may also be obtained from scientific or commercial sources. In an embodiment of the invention, antibodies are reactive against a PLM Marker if they bind with a Kₐ of greater than or equal to 10⁻⁷M.

Markers

The invention provides a set of markers correlated with pre-term labor by clustering analysis. A subset of these markers identified as useful for detection, diagnosis, prevention and therapy of pre-term labor is listed in Tables 1-6, for the respective pre-term groups. The invention also provides a method of using these markers to distinguish threatened pre-term labor that progresses to delivery from pregnancies that continue to term.

The invention provides gene marker sets that distinguish preterm labor and term labor and uses of such markers. In an aspect, the invention provides a method for classifying a sample as pre-term labor comprising detecting a difference in the expression of a first plurality of genes relative to a control, the first plurality of genes consisting of at least 5, 10, 15, 20, 25, 30, 35, 40, 45, or 50 of the genes corresponding to the markers listed in Tables 1-6. In one aspect the genes correspond to the markers listed in: Table 1 for pre-term delivery less than 48 hours from clinical presentation; Table 2 or 5, or in one aspect Table 5, for pre-term delivery less than 14 days for clinical presentation; Table 3 for pre-term delivery less than 34 weeks gestation; or in Table 4 or 6, or in one aspect Table 6, for pre-term delivery less than 37 weeks gestation. In specific aspects, the plurality of genes consists of at least 50, 100, 200, or 300 of the gene markers listed in the aforementioned Tables. In another specific aspect, the control comprises nucleic acids derived from a pool of samples from individual term patients.

Any of the markers provided herein may be used alone or with other markers of pre-term labor, or with markers for other phenotypes or conditions.

Identification of PLM Markers

As mentioned herein, the present invention provides sets of markers for detecting, diagnosing and predicting pre-term labor or onset of pre-term labor in patient samples. Generally, marker sets were identified by determining which human markers had expression patterns that correlated with pre-term labor.

Thus, the invention relates to a method of characterizing a sample, in particular a peripheral blood leukocyte sample, by detecting or quantitating in the sample one or more polynucleotides extracted from the sample that are characteristic of pre-term labor the method comprising assaying for differential expression of polynucleotides in the sample. Differential expression of the polynucleotides can be determined by micro-array analysis or by amplification of the extracted polynucleotides.

In an embodiment, a method for identifying sets or markers is provided comprising extracting and labelling target polynucleotides, and comparing the expression of all markers (genes) in a sample to the expression of all markers in a standard or control. The sample may comprise a single sample, or a pool of samples; the samples in the pool may come from different individuals. In one embodiment, the standard or control comprises target polynucleotide molecules derived from a sample from a normal individual (i.e., an individual not afflicted or pre-disposed to pre-term labor). In a particular embodiment, the standard or control is a pool of target polynucleotides derived from collected samples from a number of normal individuals.

Comparison of the patient sample and control may be accomplished by any means known in the art. By way of example, expression levels of various markers can be assessed by separation of target polynucleotides (e.g., RNA or cDNA) derived from the markers in agarose or polyacrylamide gels, followed by hybridization with marker-specific oligonucleotide probes. In the alternative, the comparison may be accomplished by the labeling of target polynucleotides followed by separation on a sequencing gel. The patient and control or standard polynucleotides can be in adjacent lanes. Expression levels can be compared visually or using a densitometer. In a particular embodiment, the expression of all markers is assessed simultaneously by hybridization to an oligonucleotide microarray. In each approach, markers meeting certain criteria are identified as associated with pre-term labor.

Markers can be selected based upon a significant difference of expression (up—or down—regulation) in a sample as compared to a standard or control. Markers can also be selected by calculation of the statistical significance (i.e., the p-value) of the correlation between the expression of the marker and pre-term labor. Both selection criteria are generally used. In an aspect of the invention, markers associated with pre-term labor are selected where the markers show more than two-fold change (increase or decrease) in expression as compared to a standard, and/or the p-value for the correlation between pre-term labor and the change in marker expression is no more than 0.01 (i.e., is statistically significant).

The expression of the identified pre-term markers can be used to identify markers that can differentiate pre-term labor into clinical types.

In particular aspects of the invention a method is provided for identifying markers associated with pre-term labor comprising:
(a) obtaining peripheral blood leukocytes from a subject;
(b) extracting polynucleotides from the peripheral blood leukocytes and producing a microarray profile of the polynucleotides; and
(c) comparing the profile with a profile for peripheral blood leukocytes from a normal individual to identify polynucleotides associated with pre-term labor.

The profile of nucleic acids can be produced by a microarray or by amplification of the nucleic acids (e.g. using PCR).

In an aspect the invention provides a method of characterizing a sample (e.g peripheral blood leukocytes) by detecting or quantitating in the sample one or more polynucleotides extracted from the sample that are characteristic of pre-term labor the method comprising assaying for differential expression of polynucleotides in the sample by microarray of polynucleotides extracted from the sample.

The preparation, use, and analysis of microarrays are described herein and are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (I 995) PCT application WO95/35505; Heller, R. A. et al. (1997) Proc. Natl. Acad. Sci. 94:2150-2155; and Heller, M. J. et al. (1997) U.S. Pat. No. 5,605,662.)

The invention also relates to a method of characterizing a sample in particular peripheral blood leukocytes by detecting or quantitating in the sample one or more polypeptides extracted from the sample that are characteristic of pre-term labor the method comprising assaying for differential expression of polypeptides in the sample. Differential expression of polypeptides can be assayed by mass spectroscopy or an antibody microarray of polypeptides extracted from the sample.

The invention relates to a method for identifying PLM Markers associated with pre-term labor comprising:
(a) obtaining a sample of peripheral blood leukocytes from a subject;
(b) extracting polypeptides from the peripheral blood leukocytes and producing a profile of the polypeptides by subjecting the polypeptides to mass spectrometry; and
(c) comparing the profile with a profile for normal peripheral blood leukocytes or for a known stage or type of pre-term labor to identify polypeptides associated with pre-term labor.

Polypeptides may be extracted from the samples in a manner known in the art. For example, polypeptides may be extracted by first digesting or disrupting cell membranes by standard methods such as detergents or homogenization in an isotonic sucrose solution, followed by ultra-centrifugation or other standard techniques.

The separated polypeptides may be digested into peptides, in particular using proteolytic enzymes such as trypsin, pepsin, subtilisin, and proteinase. For example, polypeptides may be treated with trypsin which cleaves at the sites of lysine and arginine, to provide doubly-charged peptides with a length of from about 5 to 50 amino acids. Such peptides may be particularly appropriate for mass spectrometry analysis, especially electrospray ionization mass spectrometry. Chemical reagents including cyanogen bromide may also be utilized to digest proteins.

Mass spectrometers that may be used to analyze the peptides or polypeptides include a Matrix-Assisted Laser Desorption/Ionization Time-of-Flight Mass Spectrometer (“MALDI-TOF”) (e.g. from PerSeptive Biosystems, Framingham, Mass.); an Electrospray Ionization (“ESI”) ion trap spectrometer, (e.g. from Finnigan MAT, San Jose, Calif.), an ESI quadrupole mass spectrometer (e.g. from Finnigan or Perkin-Elmer Corporation, Foster City, Calif.), a quadrupole/TOF hybrid tandem mass spectrometer, QSTAR XL (Applied Biosystems/MDS Sciex), or a Surface Enhanced Laser Desorption/Ionization (SELDI-TOF) Mass Spectrometer (e.g. from Ciphergen Biosystems Inc.).

Detection Methods

A variety of methods can be employed for the detection, diagnosis, monitoring, and prognosis of pre-term labor, onset of pre-term labor, or status of pre-term labor involving one or more PLM Markers and/or PLM Polynucleotides, and for the identification of subjects with a predisposition to pre-term labor. Such methods may, for example, utilize PLM Polynucleotides, and fragments thereof, and binding agents (e.g. antibodies) against one or more PLM Markers, including peptide fragments. In particular, the polynucleotides and antibodies may be used, for example, for (1) the detection of the presence of PLM Polynucleotide mutations, or the detection of either an over- or under-expression of PLM Polynucleotide mRNA relative to a non-pre-term state, or the qualitative or quantitative detection of alternatively spliced forms of PLM Polynucleotide transcripts which may correlate with certain conditions or susceptibility toward pre-term labor; and (2) the detection of either an over- or an under-abundance of one or more PLM Markers relative to a non-pre-term labor state or a different stage or type of injury or the presence of a modified (e.g., less than full length) PLM Marker which correlates with a pre-term labor state or a progression toward pre-term labor, or a particular type or stage of pre-term labor.

If the gene(s) represent surface antigens or secreted peptides, antibodies can be raised and standard ELISA's developed. In addition, novel automated RNA extraction can be utilized, followed by multiplex, real time RT-PCR. For example, the MagNA Pure LC & LightCycler system from Roche Diagnostic is capable of accurately quantifying RNA expression in cells within 90 minutes.

The invention contemplates a method for detecting or monitoring the stage or type of pre-term labor or onset of pre-term labor, comprising producing a profile of levels of one or more PLM Marker and/or PLM Polynucleotides, and optionally other markers associated with pre-term labor in a sample from a patient, and comparing the profile with a reference to identify a profile for the patient indicative of the stage or type of pre-term labor.

The methods described herein may be used to evaluate the probability of the presence of pre-term labor or onset of pre-term labor, for example, in a sample freshly removed from a host. Such methods can be used to detect pre-term labor and help in the diagnosis and prognosis of pre-term labor. The methods can be used to detect the potential for pre-term labor and to monitor pre-term labor or a therapy.

The invention also contemplates a method for detecting pre-term labor or onset of pre-term labor comprising producing a profile of levels of one or more PLM Marker and/or PLM Polynucleotides, and other markers associated with pre-term labor in a sample (e.g. cells) from a patient, and comparing the profile with a reference to identify a profile for the patient indicative of pre-term labor.

The methods described herein can be adapted for diagnosing and monitoring pre-term labor by detecting one or more PLM Markers or PLM Polynucleotides in biological samples from a subject. These applications require that the amount of PLM Markers or PLM Polynucleotides quantitated in a sample from a subject being tested be compared to a predetermined standard or cut-off value. The standard may correspond to levels quantitated for another sample or an earlier sample from the subject, or levels quantitated for a control sample. Levels for control samples from healthy subjects, different stages or types of pre-term labor, may be established by prospective and/or retrospective statistical studies. Healthy subjects who have no clinically evident pre-term labor or abnormalities may be selected for statistical studies. Diagnosis may be made by a finding of statistically different levels of detected PLM Markers associated with pre-term labor or PLM Polynucleotides, compared to a control sample or previous levels quantitated for the same subject.

The methods described herein may also use multiple markers for pre-term labor. Therefore, the invention contemplates a method for analyzing a biological sample for the presence of one or more PLM Markers and PLM Polynucleotides, and other markers that are specific indicators of pre-term labor. The methods described herein may be modified by including reagents to detect the additional markers.

Nucleic Acid Methods/Assays

As noted herein pre-term labor or stage or type of same may be detected based on the level of PLM Polynucleotides in a sample. Techniques for detecting polynucleotides such as polymerase chain reaction (PCR) and hybridization assays are well known in the art.

Probes may be used in hybridization techniques to detect polynucleotide markers. The technique generally involves contacting and incubating polynucleotides (e.g. recombinant DNA molecules, cloned genes) obtained from a sample from a patient or other cellular source with a probe under conditions favourable for the specific annealing of the probes to complementary sequences in the polynucleotides. After incubation, the non-annealed nucleic acids are removed, and the presence of polynucleotides that have hybridized to the probe if any are detected.

Nucleotide probes for use in the detection of nucleic acid sequences in samples may be constructed using conventional methods known in the art. Suitable probes may be based on nucleic acid sequences encoding at least 5 sequential amino acids from regions of a PLM Polynucleotide, preferably they comprise 10-30, 10-40, 15-40, 20-50, 40-80, 50-150, or 80-120 nucleotides.

A nucleotide probe may be labeled with a detectable substance such as a radioactive label that provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect PLM Polynucleotides in human samples, e.g. peripheral blood leukocytes. The nucleotide probes may also be useful in the diagnosis of pre-term labor involving one or more PLM Polynucleotides; in monitoring the progression of pre-term labor; or monitoring a therapeutic treatment.

The levels of mRNA or polynucleotides derived therefrom can be determined using hybridization methods known in the art. For example, RNA can be isolated from a sample and separated on a gel. The separated RNA can then be transferred to a solid support and nucleic acid probes representing one or more markers can be hybridized to the solid support and the amount of marker-derived RNA is determined. Such determination can be visual, or machine-aided (e.g. use of a densitometer). Dot-blot or slot-blot may also be used to determine RNA. RNA or nucleic acids derived therefrom from a sample are labeled, and then hybridized to a solid support containing oligonucleotides derived from one or more marker genes that are placed on the solid support at discrete, easily-identifiable locations. Hybridization, or the lack thereof, of the labeled RNA to the solid support oligonucleotides is determined visually or by densitometer.

The detection of PLM Polynucleotides may involve the amplification of specific gene sequences using an amplification method such as polymerase chain reaction (PCR), followed by the analysis of the amplified molecules using techniques known to those skilled in the art. Suitable primers can be routinely designed by one of skill in the art.

By way of example, at least two oligonucleotide primers may be employed in a PCR based assay to amplify a portion of a PLM Polynucleotide(s) derived from a sample, wherein at least one of the oligonucleotide primers is specific for (i.e. hybridizes to) a PLM Polynucleotide. The amplified cDNA is then separated and detected using techniques well known in the art, such as gel electrophoresis.

In order to maximize hybridization under assay conditions, primers and probes employed in the methods of the invention generally have at least about 60%, preferably at least about 75%, and more preferably at least about 90% identity to a portion of a PLM Polynucleotide; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of PLM Polynucleotide expression. For example, RNA may be isolated from a cell type or tissue known to express a PLM Polynucleotide and tested utilizing the hybridization (e.g. standard Northern analyses) or PCR techniques referred to herein. The primers and probes may be used in the above-described methods in situ i.e. directly on tissue sections (fixed and/or frozen) of patient tissue obtained from biopsies or resections.

In an aspect of the invention, a method is provided employing reverse transcriptase-polymerase chain reaction (RT-PCR), in which PCR is applied in combination with reverse transcription. Generally, RNA is extracted from a sample using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against a PLM Polynucleotide sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with a suspected pre-term labor and an individual who is not predisposed to pre-term labor. The reaction may be performed on several dilutions of cDNA spanning at least two orders of magnitude. A significant difference in expression in several dilutions of the subject sample as compared to the same dilutions of the normal sample may be considered positive for the presence of pre-term labor.

In an embodiment, the invention provides methods for determining the presence or absence of a pre-term labor in a subject comprising (a) contacting a sample obtained from the subject with oligonucleotides that hybridize to one or more PLM Polynucleotides; and (b) detecting in the sample a level of nucleic acids that hybridize to the polynucleotides relative to a predetermined cut-off value, and therefrom determining the presence or absence of pre-term labor in the subject.

The invention provides a method wherein an PLM Polynucleotide which is mRNA is detected by (a) isolating mRNA from a sample and combining the mRNA with reagents to convert it to cDNA; (b) treating the converted cDNA with amplification reaction reagents and nucleic acid primers that hybridize to one or more PLM Polynucleotides, to produce amplification products; (d) analyzing the amplification products to detect amounts of mRNA encoding PLM Polynucleotides; and (e) comparing the amount of mRNA to an amount detected against a panel of expected values for normal tissue derived using similar nucleic acid primers.

PLM Marker-positive samples or alternatively higher levels in patients compared to a control (e.g. normal tissue) may be indicative of pre-term labor or advanced pre-term labor, and/or that the patient is not responsive to or tolerant of a therapy. Alternatively, negative samples or lower levels compared to a control (e.g. normal samples or negative samples) may also be indicative of pre-term labor or advanced pre-term labor.

In another embodiment, the invention provides methods for determining the presence or absence of pre-term labor in a subject comprising (a) contacting a sample obtained from the subject with oligonucleotides that hybridize to one or more PLM Polynucleotides; and (b) detecting in the sample levels of polynucleotides that hybridize to the PLM Polynucleotides relative to a predetermined cut-off value, and therefrom determining the presence or absence of pre-term labor in the subject. In an embodiment, the PLM Polynucleotides encode one or more polynucleotides listed in Tables 1-6. In one aspect the polynucleotides correspond to those listed in: Table 1 for pre-term delivery less than 48 hours from clinical presentation; Table 2 or 5, or on one aspect Table 5, for pre-term delivery less than 14 days for clinical presentation; Table 3 for pre-term delivery less than 34 weeks gestation; or in Table 4 or 6, or in one aspect Table 6, for pre-term delivery less than 37 weeks gestation.

In a particular aspect, the invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to a PLM Polynucleotide, to produce amplification products; (d) analyzing the amplification products to detect an amount of PLM Polynucleotide mRNA; and (e) comparing the amount of mRNA to an amount detected against a panel of expected values for normal subjects derived using similar nucleic acid primers.

Marker-positive samples or alternatively higher levels, in particular significantly higher levels of PLM Polynucleotides listed in Tables 1-6 for the respective pre-term labor groups in patients compared to a control (e.g. normal) are indicative of pre-term labor.

In another particular aspect, the invention provides a method wherein PLM Polynucleotides that are mRNA are detected by (a) isolating mRNA from a sample and combining the mRNA with reagents to convert it to cDNA; (b) treating the converted cDNA with amplification reaction reagents and nucleic acid primers that hybridize to a PLM Polynucleotide, to produce amplification products; (d) analyzing the amplification products to detect an amount of PLM Polynucleotide mRNA; and (e) comparing the amount of mRNA to an amount detected against a panel of expected values for normal subjects derived using similar nucleic acid primers.

Marker-positive samples or alternatively lower levels, in particular significantly lower levels of PLM Polynucleotides listed in Tables 1-6 for the respective pre-term labor groups in patients compared to a control (e.g. normal) are indicative of pre-term labor.

Oligonucleotides or longer fragments derived from PLM Polynucleotides may be used as targets in a micro-array as described herein. The micro-array can be used to simultaneously monitor the expression levels of large numbers of genes. The micro-array can also be used to identify genetic variants, mutations, and polymorphisms. The information from the micro-array may be used to determine gene function, to understand the genetic basis of pre-term labor, to diagnose pre-term labor, and to develop and monitor the activities of therapeutic agents.

Thus, the invention also includes an array comprising one or more PLM Polynucleotides (in particular the markers listed in Tables 1-6 for the respective pre-term laborgroups, and optionally other markers. The array can be used to assay expression of PLM Polynucleotides in the array. The invention allows the quantitation of expression of one or more PLM Polynucleotides.

Micro-arrays typically contain at separate sites nanomolar quantities of individual genes, cDNAs, or ESTs on a substrate (e.g. nitrocellulose or silicon plate), or photolithographically prepared glass substrate. The arrays are hybridized to cDNA probes using conventional techniques with gene-specific primer mixes. The target polynucleotides to be analyzed are isolated, amplified and labeled, typically with fluorescent labels, radiolabels or phosphorous label probes. After hybridization is completed, the array is inserted into the scanner, where patterns of hybridization are detected. Data are collected as light emitted from the labels incorporated into the target, which becomes bound to the probe array. Probes that completely match the target generally produce stronger signals than those that have mismatches. The sequence and position of each probe on the array are known, and thus by complementarity, the identity of the target nucleic acid applied to the probe array can be determined.

Micro-arrays are prepared by selecting polynucleotide probes and immobilizing them to a solid support or surface. The probes may comprise DNA sequences, RNA sequences, copolymer sequences of DNA and RNA, DNA and/or RNA analogues, or combinations thereof. The probe sequences may be full or partial fragments of genomic DNA, or they may be synthetic oligonucleotide sequences synthesized either enzymatically in vivo, enzymatically in vitro (e.g., by PCR), or non-enzymatically in vitro.

The probe or probes used in the methods of the invention can be immobilized to a solid support or surface which may be either porous (e.g. gel), or non-porous. For example, the probes can be attached to a nitrocellulose or nylon membrane or filter covalently at either the 3′ or the 5′ end of the polynucleotide probe. The solid support may be a glass or plastic surface. In an aspect of the invention hybridization levels are measured to micro-arrays of probes consisting of a solid support on the surface of which are immobilized a population of polynucleotides.

In accordance with embodiments of the invention, a micro-array is provided comprising a support or surface with an ordered array of hybridization sites or “probes” each representing one of the markers described herein. The micro-arrays can be addressable arrays, and in particular positionally addressable arrays. Each probe of the array is typically located at a known, predetermined position on the solid support such that the identity of each probe can be determined from its position in the array. In preferred embodiments, each probe is covalently attached to the solid support at a single site.

Micro-arrays used in the present invention are preferably (a) reproducible, allowing multiple copies of a given array to be produced and easily compared with each other; (b) made from materials that are stable under hybridization conditions; (c) small, (e.g., between 1 cm² and 25 cm², between 12 cm² and 13 cm², or 3 cm²; and (d) comprise a unique set of binding sites that will specifically hybridize to the product of a single gene in a cell (e.g., to a specific mRNA, or to a specific cDNA derived therefrom). However, it will be appreciated that larger arrays may be used particularly in screening arrays, and other related or similar sequences will cross hybridize to a given binding site.

In accordance with an aspect of the invention, the micro-array is an array in which each position represents one of the markers described herein. Each position of the array can comprise a DNA or DNA analogue based on genomic DNA to which a particular RNA or cDNA transcribed from a genetic marker can specifically hybridize. A DNA or DNA analogue can be a synthetic oligomer or a gene fragment. In an embodiment, probes representing each of the PLM Markers and PLM Polynucleotides are present on the array. In a preferred embodiment, the array comprises at least 5, 10, 15, 20, 25, 30, 40, 50, 60, 70, 80, 90, 100, 125, 150, 175, 200, 225, 250, 275, or 300 of the PLM Polynucleotides (e.g. the PLM Polynucleotides of Tables 1-6. In one aspect the polynucleotides correspond to the polynucleotides listed in: Table 1 for preterm delivery less than 48 hours from clinical presentation; Table 2 or 5, or in one aspect Table 5, for pre-term delivery less than 14 days for clinical presentation; Table 3 for pre-term delivery less than 34 weeks gestation; or in Table 4 or 6, or in one aspect Table 6, for pre-term delivery less than 37 weeks gestation.).

A “probe” to which a particular polynucleotide molecule specifically hybridizes according to the invention contains a complementary genomic polynucleotide sequence. The nucleotide sequences of the probes can be about 10-200 nucleotides in length. The probes can be genomic sequences of a species of organism, such that a plurality of different probes is present, with complementary sequences capable of hybridizing to the genome of such a species of organism. In other embodiments, the probes are about 10-30, 10-40, 20-50, 40-80, 50-150, 80-120 nucleotides in length, and in particular about 60 nucleotides in length.

The probes may comprise DNA or DNA mimics (e.g., derivatives and analogues) corresponding to a portion of an organism's genome, or complementary RNA or RNA mimics. Mimics are polymers comprising subunits capable of specific, Watson-Crick-like hybridization with DNA, or of specific hybridization with RNA. The nucleic acids can be modified at the base moiety, at the sugar moiety, or at the phosphate backbone.

DNA can be obtained using standard methods such as polymerase chain reaction (PCR) amplification of genomic DNA or cloned sequences. (See, for example, in Innis et al., eds., 1990, PCR Protocols: A Guide to Methods and Applications, Academic Press Inc., San Diego, Calif.). Computer programs known in the art can be used to design primers with the required specificity and optimal amplification properties, such as Oligo version 5.0 (National Biosciences). Controlled robotic systems may be useful for isolating and amplifying nucleic acids.

Probes for the microarray can be synthesized using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between about 10 and about 500 bases, 20-100 bases, or 40-70 bases in length. Synthetic nucleic acid probes can include non-natural bases, such as, without limitation, inosine. Nucleic acid analogues such as peptide nucleic acid may be used as binding sites for hybridization. (see, e.g., Egholm et al., 1993, Nature 363:566-568; U.S. Pat. No. 5,539,083).

Probes can be selected using an algorithm that takes into account binding energies, base composition, sequence complexity, cross-hybridization binding energies, and secondary structure (see Friend et al., International Patent Publication WO 01/05935, published Jan. 25, 2001).

Positive control probes, (e.g., probes known to be complementary and hybridize to sequences in the target polynucleotides), and negative control probes, (e.g., probes known to not be complementary and hybridize to sequences in the target polynucleotides) are typically included on the array. Positive controls can be synthesized along the perimeter of the array or synthesized in diagonal stripes across the array. A reverse complement for each probe can be next to the position of the probe to serve as a negative control.

The probes can be attached to a solid support or surface, which may be made from glass, plastic (e.g., polypropylene, nylon), polyacrylamide, nitrocellulose, gel, or other porous or nonporous material. The probes can be printed on surfaces such as glass plates (see Schena et al., 1995, Science 270:467-470). This method may be particularly useful for preparing microarrays of cDNA (See also, DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

High-density oligonucleotide arrays containing thousands of oligonucleotides complementary to defined sequences, at defined locations on a surface can be produced using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). Using these methods oligonucleotides (e.g., 60-mers) of known sequence are synthesized directly on a surface such as a derivatized glass slide. The array produced may be redundant, with several oligonucleotide molecules per RNA.

Microarrays can be made by other methods including masking (Maskos and Southern, 1992, Nuc. Acids. Res. 20:1679-1684).

In an embodiment, microarrays of the present invention are produced by synthesizing polynucleotide probes on a support wherein the nucleotide probes are attached to the support covalently at either the 3′ or the 5′ end of the polynucleotide.

The invention provides micro-arrays comprising a disclosed marker set. In one embodiment, the invention provides a micro-array for distinguishing pre-term samples comprising a positionally-addressable array of polynucleotide probes bound to a support, the polynucleotide probes comprising a plurality of polynucleotide probes of different nucleotide sequences, each of the different nucleotide sequences comprising a sequence complementary and hybridizable to a plurality of genes, the plurality consisting of at least 5, 10, 15, or 20 of the genes corresponding to the markers listed in Tables 1-6. An aspect of the invention provides micro-arrays comprising at least 20, 25, 30, 35, 40, 45, 50, 75, 100, 125, 150, 175, 200, 225, 250, 275, or 300 of the marker genes listed in Tables 1-6 for the respective pre-term labor groups. In a particular embodiment, a micro-array comprises the genes listed in Tables 1-6. In one aspect the genes correspond to the markers listed in: Table 1 for pre-term delivery less than 48 hours from clinical presentation; Table 2 or 5, or in one aspect Table 5, for pre-term delivery less than 14 days for clinical presentation; Table 3 for pre-term delivery less than 34 weeks gestation; or in Table 4 or 6, or in one aspect Table 6, for pre-term delivery less than 37 weeks gestation.

The invention provides gene marker sets that distinguish preterm labor and term labor and uses therefor. In an aspect, the invention provides a method for classifying a sample as pre-term labor comprising detecting a difference in the expression of a first plurality of genes relative to a control, the first plurality of genes consisting of at least 5, 10, 15, or 20, of the genes corresponding to the markers listed in Tables 1-6. In one aspect the genes correspond to the markers listed in: Table 1 for pre-term delivery less than 48 hours from clinical presentation; Table 2 or 5, or in one aspect Table 5, for pre-term delivery less than 14 days for clinical presentation; Table 3 for pre-term delivery less than 34 weeks gestation; or in Table 4 or 6, or in one aspect Table 6, for pre-term delivery less than 37 weeks gestation.

In specific aspects, the plurality of genes consists of at least 20, 25, 30, 35, 40, 45, 50, 75, 100, 125, 150, 175, 200, 225, 250, 275, or 3000f the gene markers listed in Tables 1-6. In one aspect the genes correspond to the markers listed in: Table 1 for pre-term delivery less than 48 hours from clinical presentation; Table 2 or 5, or in one aspect Table 5, for pre-term delivery less than 14 days for clinical presentation; Table 3 for pre-term delivery less than 34 weeks gestation; or in Table 4 or 6, or in one aspect Table 6, for pre-term delivery less than 37 weeks gestation. In another specific aspect, the control comprises nucleic acids derived from a pool of samples from individual term patients.

The invention provides a method for classifying a sample as pre-term labor by calculating the similarity between the expression of at least 5, 10, 15, 20, 25, 30, 40, or 50 of the markers listed in Tables 1-6 for the respective pre-term labor groups, in the sample to the expression of the same markers in a term pool, comprising the steps of:
(a) labeling nucleic acids derived from a sample, with a first fluorophore to obtain a first pool of fluorophore-labeled nucleic acids;
(b) labeling with a second fluorophore a first pool of nucleic acids derived from two or more preterm samples, and a second pool of nucleic acids derived from two or more term samples;
(c) contacting the first fluorophore-labeled nucleic acid and the first pool of second fluorophore-labeled nucleic acid with a first micro-array under conditions such that hybridization can occur, and contacting the first fluorophore-labeled nucleic acid and the second pool of second fluorophore-labeled nucleic acid with a second microarray under conditions such that hybridization can occur, detecting at each of a plurality of discrete loci on the first microarray a first flourescent emission signal from the first fluorophore-labeled nucleic acid and a second fluorescent emission signal from the first pool of second fluorophore-labeled genetic matter that is bound to the first microarray and detecting at each of the marker loci on the second microarray the first fluorescent emission signal from the first fluorophore-labeled nucleic acid and a third fluorescent emission signal from the second pool of second fluorophore-labeled nucleic acid;
(d) determining the similarity of the sample to the term and preterm pools by comparing the first fluorescence emission signals and the second fluorescence emission signals, and the first emission signals and the third fluorescence emission signals; and
(e) classifying the sample as preterm where the first fluorescence emission signals are more similar to the second fluorescence emission signals than to the third fluorescent emission signals, and classifying the sample as term where the first fluorescence emission signals are more similar to the third fluorescence emission signals than to the second fluorescent emission signals, wherein the first microarray and the second microarray are similar to each other, exact replicas of each other, or are identical, and wherein the similarity is defined by a statistical method such that the sample and control are similar where the p value of the similarity is less than 0.01, more particularly less than 0.001.

In an embodiment, the array can be used to monitor the time course of expression of one or more PLM Polynucleotides in the array. This can occur in various biological contexts such as progression of pre-term labor.

Arrays are also useful for ascertaining differential expression patterns of PLM Polynucleotides as described herein, and optionally other markers, in normal and abnormal samples. This may provide a battery of nucleic acids that could serve as molecular targets for diagnosis or therapeutic intervention.

Protein Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of pre-term labor or stage or type of pre-term labor in a subject may be determined by (a) contacting a sample from the subject with a binding agent; (b) detecting in the sample a level of PLM polypeptide(s) that binds to the binding agent; and (c) comparing the level of PLM Polypeptide(s) with a predetermined standard or cut-off value.

In particular embodiments of the invention, the binding agent is an antibody. Antibodies specifically reactive with one or more PLM Marker, or derivatives, such as enzyme conjugates or labeled derivatives, may be used to detect one or more PLM Marker in various samples (e.g. biological materials). They may be used as diagnostic or prognostic reagents and they may be used to detect abnormalities in the level of expression of one or more PLM Marker, or abnormalities in the structure, and/or temporal, tissue, cellular, or subcellular location of one or more PLM Marker. Antibodies may also be used to screen potentially therapeutic compounds in vitro to determine their effects on pre-term labor involving one or more PLM Markers, and other conditions. In vitro immunoassays may also be used to assess or monitor the efficacy of particular therapies.

In an aspect, the invention provides a diagnostic method for monitoring or diagnosing pre-term labor in a subject by quantitating one or more PLM Markers in a biological sample from the subject comprising reacting the sample with antibodies specific for one or more PLM Markers, which are directly or indirectly labeled with detectable substances and detecting the detectable substances. In a particular embodiment of the invention, PLM Markers are quantitated or measured.

In an aspect of the invention, a method for detecting pre-term labor is provided comprising:
(a) obtaining a sample suspected of containing one or more PLM Markers associated with pre-term labor;
(b) contacting said sample with antibodies that specifically bind to the PLM Markers under conditions effective to bind the antibodies and form complexes;
(c) measuring the amount of PLM Markers present in the sample by quantitating the amount of the complexes; and
(d) comparing the amount of PLM Markers present in the samples with the amount of PLM Markers in a control, wherein a change or significant difference in the amount of PLM Markers in the sample compared with the amount in the control is indicative of pre-term labor.

In an embodiment, the invention contemplates a method for monitoring the progression of pre-term labor in an individual, comprising:
(a) contacting antibodies which bind to one or more PLM Markers with a sample from the individual so as to form complexes comprising the antibodies and one or more PLM Markers in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of pre-term labor in said individual.

The amount of complexes may also be compared to a value representative of the amount of the complexes from an individual not at risk of, or afflicted with, a pre-term labor at different stages. A significant difference in complex formation may be indicative of advanced pre-term labor, or an unfavourable prognosis.

In an embodiment of methods of the invention, the PLM Markers encoded by the polynucleotides in Tables 1-6 (in one aspect the genes correspond to the markers listed in: Table 1 for pre-term delivery less than 48 hours from clinical presentation; Table 2 or 5, or in one aspect Table 5, for pre-term delivery less than 14 days for clinical presentation; Table 3 for pre-term delivery less than 34 weeks gestation; or in Table 4 or 6, or in one aspect Table 6, for pre-term delivery less than 37 weeks gestation) are detected in samples and higher levels, in particular significantly higher levels compared to a control (normal) is indicative of pre-term labor.

In a further embodiment of methods of the invention, the PLM Markers encoded by the polynucleotides in Tables 1-6 (in one aspect the genes correspond to the markers listed in: Table 1 for pre-term delivery less than 48 hours from clinical presentation; Table 2 or 5, or in one aspect Table 5, for pre-term delivery less than 14 days for clinical presentation; Table 3 for pre-term delivery less than 34 weeks gestation; or in Table 4 or 6, or in one aspect Table 6, for pre-term delivery less than 37 weeks gestation) are detected in samples and lower levels, in particular significantly lower levels compared to a control (normal) is indicative of pre-term labor.

A particular embodiment of the invention comprises the following steps
(a) incubating a biological sample with first antibodies specific for one or more PLM Markers which are directly or indirectly labeled with a detectable substance, and second antibodies specific for one or more PLM Markers which are immobilized;
(b) detecting the detectable substance thereby quantitating PLM Markers in the biological sample; and
(c) comparing the quantitated PLM Markers with levels for a predetermined standard.

The standard may correspond to levels quantitated for samples from control subjects without pre-term labor (normal), with a different stage of pre-term labor, or from other samples of the subject. In an embodiment, increased levels of PLM Markers as compared to the standard may be indicative of pre-term labor. In another embodiment, lower levels of PLM Markers as compared to the standard may be indicative of pre-term labor.

Embodiments of the methods of the invention involve (a) reacting a biological sample from a subject with antibodies specific for one or more PLM Markers which are directly or indirectly labelled with an enzyme; (b) adding a substrate for the enzyme wherein the substrate is selected so that the substrate, or a reaction product of the enzyme and substrate forms fluorescent complexes; (c) quantitating one or more PLM Markers in the sample by measuring fluorescence of the fluorescent complexes; and (d) comparing the quantitated levels to levels obtained for other samples from the subject patient, or control subjects.

In another embodiment the quantitated levels are compared to levels quantitated for control subjects (e.g. normal) without pre-term labor wherein an increase in PLM Marker levels compared with the control subjects is indicative of pre-term labor.

In further embodiment the quantitated levels are compared to levels quantitated for control subjects (e.g. normal) without pre-term labor wherein a decrease in PLM Marker levels compared with the control subjects is indicative of pre-term labor.

Antibodies may be used in any known immunoassays that rely on the binding interaction between antigenic determinants of one or more PLM Marker and the antibodies. Immunoassay procedures for in vitro detection of antigens in fluid samples are also well known in the art. [See for example, Paterson et al., Int. J. Can. 37:659 (1986) and Burchell et al., Int. J. Can. 34:763 (1984) for a general description of immunoassay procedures]. Qualitative and/or quantitative determinations of one or more PLM Marker in a sample may be accomplished by competitive or non-competitive immunoassay procedures in either a direct or indirect format. Detection of one or more PLM Marker using antibodies can be done utilizing immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. These terms are well understood by those skilled in the art. A person skilled in the art will know, or can readily discern, other immunoassay formats without undue experimentation.

In an embodiment of the invention, an immunoassay for detecting more than one PLM Marker in a biological sample comprises contacting binding agents that specifically bind to PLM Markers in the sample under conditions that allow the formation of first complexes comprising a binding agent and PLM Markers and determining the presence or amount of the complexes as a measure of the amount of PLM Markers contained in the sample. In a particular embodiment, the binding agents are labeled differently or are capable of binding to different labels.

Binding agents (e.g. antibodies) may be used in immunohistochemical analyses, for example, at the cellular and sub-subcellular level, to detect one or more PLM Markers, to localize them to particular cells and tissues, and to specific subcellular locations, and to quantitate the level of expression.

Immunohistochemical methods for the detection of antigens in tissue samples are well known in the art. For example, immunohistochemical methods are described in Taylor, Arch. Pathol. Lab. Med. 102:112 (1978). Briefly, in the context of the present invention, a tissue sample obtained from a subject suspected of having a pre-term labor is contacted with antibodies, preferably monoclonal antibodies recognizing one or more PLM Markers. The site at which the antibodies are bound is determined by selective staining of the sample by standard immunohistochemical procedures. The same procedure may be repeated on the same sample using other antibodies that recognize one or more PLM Markers. Alternatively, a sample may be contacted with antibodies against one or more PLM Markers simultaneously, provided that the antibodies are labeled differently or are able to bind to a different label.

Antibodies specific for one or more PLM Marker may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways an antibody can be detectably labeled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steriod isomerase, yeast alcohol dehydrogenase, alpha-glycerophosphate, triose phosphate isomerase, asparaginase, glucose oxidase, and acetylcholine esterase.

A bioluminescent compound may also be used as a detectable substance. Bioluminescence is a type of chemiluminescence found in biological systems where a catalytic protein increases the efficiency of the chemiluminescent reaction. The presence of a bioluminescent molecule is determined by detecting the presence of luminescence. Examples of bioluminescent detectable substances are luciferin, luciferase and aequorin.

Indirect methods may also be employed in which the primary antigen-antibody reaction is amplified by the introduction of a second antibody, having specificity for the antibody reactive against one or more PLM Markers. By way of example, if the antibody having specificity against one or more PLM Markers is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect one or more PLM Markers. Generally, antibodies may be labeled with detectable substances and one or more PLM Markers may be localised in tissues and cells based upon the presence of the detectable substances.

In the context of the methods of the invention, the sample, binding agents (e.g. antibodies specific for one or more PLM Markers), or one or more PLM Markers may be immobilized on a carrier or support. Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling. An antibody may be indirectly immobilized using a second antibody specific for the antibody. For example, mouse antibody specific for a PLM Marker may be immobilized using sheep anti-mouse IgG Fc fragment specific antibody coated on the carrier or support.

Where a radioactive label is used as a detectable substance, one or more PLM Marker may be localized by radioautography. The results of radioautography may be quantitated by determining the density of particles in the radioautographs by various optical methods, or by counting the grains.

One or more PLM Marker antibodies may also be indirectly labelled with an enzyme using ligand binding pairs. For example, the antibodies may be conjugated to one partner of a ligand binding pair, and the enzyme may be coupled to the other partner of the ligand binding pair. Representative examples include avidin-biotin, and riboflavin-riboflavin binding protein. In an embodiment, the antibodies are biotinylated, and the enzyme is coupled to streptavidin. In another embodiment, an antibody specific for PLM Marker antibody is labeled with an enzyme.

Computer Systems

The analytic methods described herein can be implemented by use of computer systems and methods described below and known in the art. Thus the invention provides computer readable media comprising one or more PLM Markers, and/or PLM Polynucleotides, and optionally other markers (e.g. markers of pre-term labor). “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on one or more PLM Markers, and/or PLM Polynucleotides, and optionally other markers.

A variety of data processor programs and formats can be used to store information on one or more PLM Markers, and/or PLM Polynucleotides, and other markers on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of data processor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention also provides in an electronic system and/or in a network, a method for determining whether a subject has pre-term labor or a pre-disposition to pre-term labor, comprising determining the presence or absence of one or more PLM Markers, and/or PLM Polynucleotides, and optionally other markers, and based on the presence or absence of the one or more PLM Markers, and/or LI Polynucleotides, and optionally other markers, determining whether the subject has pre-term labor, or a pre-disposition to pre-term labor, and optionally recommending a procedure or treatment.

The invention further provides in a network, a method for determining whether a subject has pre-term labor or a pre-disposition to pre-term labor comprising: (a) receiving phenotypic information on the subject and information on one or more PLM Markers, and/or PLM Polynucleotides, and optionally other markers associated with samples from the subject; (b) acquiring information from the network corresponding to the one or more PLM Markers, and/or PLM Polynucleotides, and optionally other markers; and (c) based on the phenotypic information and information on the one or more PLM Markers, and/or PLM Polynucleotides, and optionally other markers, determining whether the subject has pre-term labor or a pre-disposition to pre-term labor; and (d) optionally recommending a procedure or treatment.

The invention still further provides a system for identifying selected records that identify pre-term labor. A system of the invention generally comprises a digital computer; a database server coupled to the computer; a database coupled to the database server having data stored therein, the data comprising records of data comprising one or more PLM Markers, and/or PLM Polynucleotides, and optionally other markers, and a code mechanism for applying queries based upon a desired selection criteria to the data file in the database to produce reports of records which match the desired selection criteria.

In an aspect of the invention a method is provided for detecting cells or tissues associated with pre-term labor using a computer having a processor, memory, display, and input/output devices, the method comprising the steps of:
(a) creating records of one or more PLM Markers, and/or PLM Polynucleotides, and optionally other markers, identified in a sample suspected of containing PLM Markers, and/or PLM Polynucleotides associated with pre-term labor;
(b) providing a database comprising records of data comprising one or more PLM Markers, and/or PLM Polynucleotides, and optionally other markers of pre-term labor; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from cells or tissue that are associated with pre-term labor.

The invention contemplates a business method for determining whether a subject has pre-term labor or a pre-disposition to pre-term labor comprising: (a) receiving phenotypic information on the subject and information on one or more PLM Markers, and/or PLM Polynucleotides, and optionally other markers, associated with samples from the subject; (b) acquiring information from a network corresponding to one or more PLM Markers, and/or PLM Polynucleotides, and optionally other markers; and (c) based on the phenotypic information, information on one or more PLM Markers, and/or PLM Polynucleotides encoding the markers, and optionally other markers, and acquired information, determining whether the subject has pre-term labor or a pre-disposition to a pre-term labor; and (d) optionally recommending a procedure or treatment.

In an aspect of the invention, the computer systems, components, and methods described herein are used to monitor pre-term labor or determine the stage or type of pre-term labor.

Screening Methods

The invention also contemplates methods for evaluating test agents or compounds for their ability to prevent, inhibit or reduce pre-term labor, potentially contribute to pre-term labor, or inhibit or enhance a type of pre-term labor. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The agents or compounds may be endogenous physiological compounds or natural or synthetic compounds.

The invention provides a method for assessing the potential efficacy of a test agent for inhibiting pre-term labor or onset of pre-term labor in a patient, the method comprising comparing:
(a) levels of one or more PLM Markers, and/or PLM Polynucleotides, and optionally other markers in a first sample obtained from a patient and exposed to the test agent; and
(b) levels of one or more PLM Markers and/or PLM Polynucleotides, and optionally other markers in a second sample obtained from the patient, wherein the sample is not exposed to the test agent, wherein a significant difference in the levels of expression of one or more PLM Markers, and/or PLM Polynucleotides, and optionally the other markers, in the first sample, relative to the second sample, is an indication that the test agent is potentially efficacious for inhibiting pre-term labor or onset of pre-term labor in the patient.

The first and second samples may be portions of a single sample obtained from a patient or portions of pooled samples obtained from a patient.

In an aspect, the invention provides a method of selecting an agent for inhibiting pre-term labor or onset of pre-term labor in a patient comprising:
(a) obtaining a sample from the patient;
(b) separately maintaining aliquots of the sample in the presence of a plurality of test agents;
(c) comparing one or more PLM Markers, and/or PLM Polynucleotides, and optionally other markers, in each of the aliquots; and
(d) selecting one of the test agents which alters the levels of one or more PLM Markers, and/or PLM Polynucleotides, and optionally other markers in the aliquot containing that test agent, relative to other test agents.

Still another aspect of the present invention provides a method of conducting a drug discovery business comprising:
(a) providing one or more methods or assay systems for identifying agents that inhibit, prevent or reduce pre-term labor, onset of pre-term labor, or affect a stage or type of pre-term labor in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test compound to contribute to pre-term labor or onset of pre-term labor comprising:
(a) maintaining separate aliquots of cells or tissues from a patient with pre-term labor in the presence and absence of the test compound; and
(b) comparing one or more PLM Markers, and/or PLM Polynucleotides, and optionally other markers in each of the aliquots.

A significant difference between the levels of the markers in the aliquot maintained in the presence of (or exposed to) the test compound relative to the aliquot maintained in the absence of the test compound, indicates that the test compound possesses the potential to contribute to pre-term labor or onset of pre-term labor.

Kits

The invention also contemplates kits for carrying out the methods of the invention. Kits may typically comprise two or more components required for performing a diagnostic assay. Components include but are not limited to compounds, reagents, containers, and/or equipment.

The methods described herein may be performed by utilizing pre-packaged diagnostic kits comprising one or more specific PLM Marker, PLM Polynucleotide, or binding agent (e.g. antibody) described herein, which may be conveniently used, e.g., in clinical settings to screen and diagnose patients and to screen and identify those individuals exhibiting a predisposition to developing pre-term labor.

In an embodiment, a container with a kit comprises a binding agent as described herein. By way of example, the kit may contain antibodies or antibody fragments which bind specifically to epitopes of one or more PLM Markers, and optionally other markers, antibodies against the antibodies labelled with an enzyme, and a substrate for the enzyme. The kit may also contain microtiter plate wells, standards, assay diluent, wash buffer, adhesive plate covers, and/or instructions for carrying out a method of the invention using the kit.

In an aspect of the invention, the kit includes antibodies or fragments of antibodies which bind specifically to an epitope of one or more markers encoding polynucleotides listed in Tables 1-6 (in one aspect the genes correspond to the markers listed in: Table 1 for pre-term delivery less than 48 hours from clinical presentation; Table 2 or 5, or in one aspect Table 5, for pre-term delivery less than 14 days for clinical presentation; Table 3 for pre-term delivery less than 34 weeks gestation; or in Table 4 or 6, or in one aspect Table 6, for pre-term delivery less than 37 weeks gestation) and means for detecting binding of the antibodies to their epitope associated with pre-term labor, either as concentrates (including lyophilized compositions), which may be further diluted prior to use or at the concentration of use, where the vials may include one or more dosages.

A kit may be designed to detect the level of polynucleotides encoding one or more PLM Polynucleotides in a sample. In an embodiment, the polynucleotides encode one or more polynucleotides listed in Tables 1-6 (in one aspect the genes correspond to the markers listed in: Table 1 for pre-term delivery less than 48 hours from clinical presentation; Table 2 or 5, or in one aspect Table 5, for pre-term delivery less than 14 days for clinical presentation; Table 3 for pre-term delivery less than 34 weeks gestation; or in Table 4 or 6, or in one aspect Table 6, for pre-term delivery less than 37 weeks gestation. Such kits generally comprise at least one oligonucleotide probe or primer, as described herein, that hybridizes to a PLM Polynucleotide. Such an oligonucleotide may be used, for example, within a PCR or hybridization procedure.

The invention provides a kit containing a micoarray described herein ready for hybridization to target PLM Polynucleotides, plus software for the data analysis of the results. The software to be included with the kit comprises data analysis methods, in particular mathematical routines for marker discovery, including the calculation of correlation coefficients between clinical categories and marker expression. The software may also include mathematical routines for calculating the correlation between sample marker expression and control marker expression, using array-generated fluorescence data, to determine the clinical classification of the sample.

The reagents suitable for applying the screening methods of the invention to evaluate compounds may be packaged into convenient kits described herein providing the necessary materials packaged into suitable containers.

The invention relates to a kit for assessing the suitability of each of a plurality of test compounds for inhibiting pre-term labor or onset of pre-term labor in a patient. The kit comprises reagents for assessing one or more PLM Markers or PLM Polynucleotides, and optionally a plurality of test agents or compounds.

The invention contemplates a kit for assessing the presence of cells and tissues associated with pre-term labor or onset of pre-term labor, wherein the kit comprises antibodies specific for one or more PLM Markers, or primers or probes for PLM Polynucleotides, and optionally probes, primers or antibodies specific for other markers associated with pre-term labor (e.g. fibronectin).

Additionally the invention provides a kit for assessing the potential of a test compound to contribute to pre-term labor. The kit comprises cells and tissues associated with pre-term labor or onset of pre-term labor and reagents for assessing one or more PLM Markers, PLM Polynucleotides, and optionally other markers associated with pre-term labor.

Therapeutic Applications

One or more PLM Markers may be targets for immunotherapy. Immunotherapeutic methods include the use of antibody therapy. In one aspect, the invention provides one or more PLM Marker antibodies that may be used to prevent onset of pre-term labor associated with the marker. In another aspect, the invention provides a method of preventing, inhibiting or reducing pre-term labor or the onset of pre-term labor, comprising administering to a patient an antibody which binds specifically to one or more PLM Markers in an amount effective to prevent, inhibit, or reduce pre-term labor or the onset of pre-term labor.

The methods of the invention contemplate the administration of single PLM Marker antibodies as well as combinations, or “cocktails”, of different individual antibodies such as those recognizing different epitopes of other markers. Such cocktails may have certain advantages inasmuch as they contain antibodies that bind to different epitopes of PLM Markers and/or exploit different effector mechanisms. Such antibodies in combination may exhibit synergistic therapeutic effects. In addition, the administration of one or more PLM Marker specific antibodies may be combined with other therapeutic agents. The PLM Marker specific antibodies may be administered in their “naked” or unconjugated form, or may have therapeutic agents conjugated to them.

The PLM Marker specific antibodies used in the methods of the invention may be formulated into pharmaceutical compositions comprising a carrier suitable for the desired delivery method. Suitable carriers include any material which when combined with the antibodies retains the function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16.sup.th Edition, A. Osal., Ed., 1980).

One or more PLM Marker specific antibody formulations may be administered via any route capable of delivering the antibodies to the site or injury. Routes of administration include, but are not limited to, intravenous, intraperitoneal, intramuscular, intradermal, and the like. Antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration at an effective dose. Dosages will depend upon various factors generally appreciated by those of skill in the art, including the etiology of the pre-term labor, stage of pre-term labor, the binding affinity and half life of the antibodies used, the degree of PLM Marker expression in the patient, the desired steady-state antibody concentration level, frequency of treatment, and the influence of any therapeutic agents used in combination with a treatment method of the invention. A determining factor in defining the appropriate dose is the amount of a particular antibody necessary to be therapeutically effective in a particular context. Repeated administrations may be required to achieve a desired effect. Direct administration of one or more PLM Marker antibodies is also possible and may have advantages in certain situations.

Patients may be evaluated for markers in-order to assist in the determination of the most effective dosing regimen and related factors. The assay methods described herein, or similar assays, may be used for quantitating PLM Marker levels in patients prior to treatment. Such assays may also be used for monitoring throughout therapy, and may be useful to gauge therapeutic success in combination with evaluating other parameters such as levels of PLM Markers.

PLM Polynucleotides associated with pre-term labor can be turned off by transfecting a cell or tissue with vectors that express high levels of a desired PLM Polynucleotide. Such constructs can inundate cells with untranslatable sense or antisense sequences. Even in the absence of integration into the DNA, such vectors may continue to transcribe RNA molecules until all copies are disabled by endogenous nucleases.

Vectors derived from retroviruses, adenovirus, herpes or vaccinia viruses, or from various bacterial plasmids, may be used to deliver PLM Polynucleotides to a targeted organ, tissue, or cell population. Methods well known to those skilled in the art may be used to construct recombinant vectors that will express PLM Polynucleotides such as antisense. (See, for example, the techniques described in Sambrook et al (supra) and Ausubel et al (supra).)

Methods for introducing vectors into cells or tissues include those methods discussed herein and which are suitable for in vivo, in vitro and ex vivo therapy. For example, delivery by transfection and by liposome are well known in the art.

Modifications of gene expression can be obtained by designing antisense molecules, DNA, RNA or PNA, to the regulatory regions of a PLM Polynucleotide, i.e., the promoters, enhancers, and introns. Preferably, oligonucleotides are derived from the transcription initiation site, e.g. between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA are reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of PLM Polynucleotides.

Specific ribozyme cleavage sites within any potential RNA target may initially be identified by scanning the target molecule for ribozyme cleavage sites which include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

One or more PLM Markers and PLM Polynucleotides (e.g. down-regulated PLM Markers and PLM Polynucleotides), and fragments thereof, and compounds or agents identified using a method of the invention may be used to prevent, treat, or reduce pre-term labor or onset of pre-term labor in a subject. The markers or polynucleotides may be formulated into compositions for administration to subjects with a pre-disposition for or suffering from pre-term labor. Therefore, the present invention also relates to a composition comprising one or more PLM Markers or PLM Polynucleotides, or a fragment thereof, and a pharmaceutically acceptable carrier, excipient or diluent. A method for treating or preventing pre-term labor in a subject is also provided comprising administering to a patient in need thereof, one or more PLM Markers or PLM Polynucleotides, an agent or compound identified using a method of the invention, or a composition of the invention.

The invention further provides a method of preventing, inhibiting, or reducing pre-term labor in a patient comprising:
(a) obtaining a sample comprising tissue or cells associated with or diagnostic for pre-term labor from the patient;
(b) separately maintaining aliquots of the sample in the presence of a plurality of test agents;
(c) comparing levels of one or more PLM Markers, and/or PLM Polynucleotides in each aliquot;
(d) administering to the patient at least one of the test agents which alters the levels of the PLM Markers, and/or PLM Polynucleotides in the aliquot containing that test agent, relative to the other test agents.

An active therapeutic substance described herein may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment (e.g. antenatal glucocorticoids or tocolysis). The compositions of the invention may be administered concurrently, separately, or sequentially with other therapeutic agents or therapies.

The therapeutic activity of compositions and agents/compounds identified using a method of the invention and may be evaluated in vivo using a suitable animal model.

The methods of the invention for use on subjects/individuals/patients contemplate prophylactic as well as therapeutic or curative use. Typical subjects for treatment include persons susceptible to, suffering from or that have suffered pre-term labor. In embodiments of the invention, the methods and compositions described herein are used prophylactically to prevent development of pre-term labor.

The following non-limiting example is illustrative of the present invention:

### EXAMPLE

A cDNA micro-array analysis was used to define gene expression profiles from peripheral blood leukocytes of symptomatic women in threatened pre-term labor. A novel “non-biased” approach was used that surveys the whole genome for potential markers of imminent pre-term birth. The cDNA micro-array is a non-biased screen of the entire genome or at least the number of genes represented on the cDNA chipset.

This technique makes no predictions as to which gene or more likely gene clusters expressed by maternal mononuclear leukocytes will be predictive of imminent pre-term delivery. For the development of a diagnostic test there is also no requirement that the role of the gene product be known or even that it be identified beyond its sequence within the genome database, merely that the marker predicts imminent pre-term delivery with a high degree of sensitivity and specificity. In many ways this is an ideal use of micro-array technology. Moreover, unlike fetal fibronectin, this unbiased approach has the potential to discriminate between pre-term labors resulting from different etiologies (e.g. infection vs. fetal stress vs. myometrial activation) based on the differential gene expression profiles that might be induced. This may ultimately provide the opportunity for the provision of an individualized therapeutic strategy that is likely to be a more effective approach to the management of pre-term labor.

A diagnostic test based on the gene expression profiles identified in the peripheral blood leukocytes of symptomatic women in threatened pre-term labor will have a better ability to predict imminent pre-term birth ability than fetal fibronectin for a number of reasons. First, it is likely that a diagnostic test based on an expression profile of a specifically selected gene cluster will have a higher positive predictive value than that reported with fetal fibronectin (14%) when it is assessed in symptomatic women in threatened pre-term labor. Complex statistical analysis and predictive modeling will allow the selection of a gene cluster that possesses the best combination of sensitivity, specificity, and positive and negative predictive values for the dataset available from the study. Second, a test based on a maternal blood sample is unlikely to have the multiple contraindications that are a practical issue with FFN testing (cervical dilation greater than 3 cm, pre-term premature rupture of fetal membranes, moderate or heavy bleeding, cervical examination, coitus or transvaginal ultrasound within the previous 24 hours, obstetric cream or KY jelly) which preclude its use in up to 80% of women who present to tertiary centers with threatened pre-term labor.

Rationale for Mononuclear Leukocytes as a Diagnostic Marker of True Pre-Term Labor

While the molecular information required to diagnose true pre-term labor likely resides within the myometrium itself, an acceptable diagnostic test must use a more non-invasive approach. The source of RNA for the micro-array analysis is maternal peripheral blood cells, essentially mononuclear leukocytes. Peripheral leukocytes can be used to monitor a variety of pathophysiologic situations, including the progression of labor (62) and myometrial responsiveness to β-adrenergic tocolysis (63). Mononuclear leukocytes from women in active labor exhibit a significant attenuation of β-adrenergic receptor function due to reduced adenyl cyclase activity. This effect could be induced in mononuclear leukocytes from non-laboring women by pre-incubation with PGE but not oxytocin or PGF. It has been shown that, (1) mononuclear leukocyte and myometrial p-receptor number are positively correlated, (2) the mechanism responsible for desensitization of mononuclear leukocytes from pregnant women involves a down-regulation of the P-receptor with post receptor mechanisms remaining fully functional, (3) the process of mononuclear leukocyte desensitization can be monitored temporally during administration of tocolytic therapy to women.

Evaluation of Women Presenting with Threatened Pre-Term Labour:

Methods

Patient Recruitment:

Blood samples were analyzed from women who present with symptoms of threatened idiopathic pre-term labour. To select a homogenous group of women with idiopathic pre-term labour, detailed inclusion and exclusion criteria were determined. The inclusion criteria include: 1) 24 to 37 week's gestation; 2) regular uterine contractions; 3) cervical dilation <4 cm; 4) intact fetal membranes. The exclusion criteria include: 1) antepartum haemorrhage [abruptio placenta, placenta praevia]; 2) pre-term pre-labour rupture of membranes; 3) clinical chorioamnionitis [febrile (>37.5° C.), uterine tenderness, mother systemically unwell, fetal tachycardia]; 4) fetal anomaly; 5) preeclampsia; 6) intrauterine growth restriction; 7) diabetes or gestational diabetes; 8) maternal medical condition; 9) multi-fetal pregnancy.

Sample Processing

Detailed maternal data was collected from the clinical record. Maternal blood samples (12.5 mL) was collected into five PAXgene blood collection tubes (Qiagen, Mississauga, Ontario, Canada) designed specifically for the extraction of RNA from whole blood. Preliminary studies determined that approximately 53 μg of high quality RNA (OD 260:280 ratio 2.03+0.22 {mean ±SD}) can be extracted from each 12.5 mL maternal blood sample. This amount was adequate to 1) prepare four micro-arrays on each sample, and 2) to confirm results using real time polymerase chain reaction (RT-PCR). RNA extraction samples were concentrated with MinElute columns (Qiagen, Mississauga, Ontario, Canada) to obtain optimum RNA concentrations for micro-array analysis and their integrity was assessed using the Agilent 2100 Bioanalyser system prior to micro-array preparation.

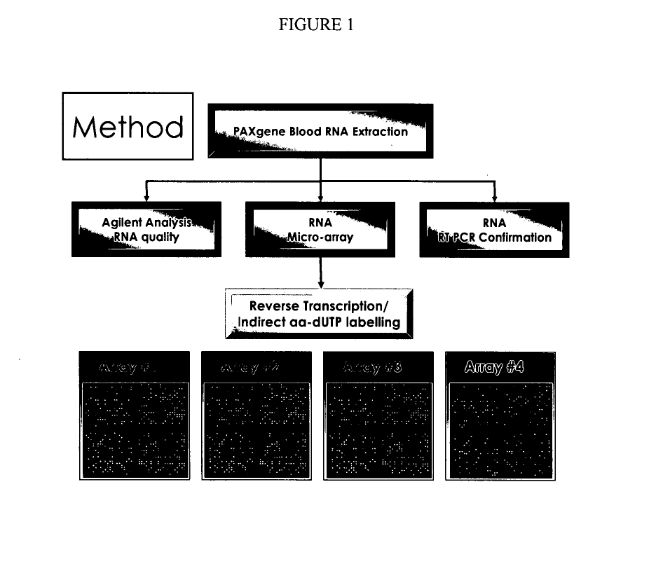

Maternal RNA samples from each woman were used to generate two -four micro-arrays (i.e. 4 technical replicates) on each example. RNA from each woman was conserved for reverse transcription PCT confirmation of the microarray results. To control for variations during reverse transcription, four separate reverse transcriptions were performed. On each array the sample was compared to human universal reference RNA (Stratagene, California, USA). An indirect (amino-allyl) labeling protocol was utilized to reduce dye incorporation bias. Dye swapping was performed on one of the four micro-arrays performed on each sample to further reduce bias induced by variable dye incorporation. The micro-array protocol used in the study was validated. In brief, cDNA was prepared by reverse transcription of both sample RNA and reference RNA using amino allyl labeled dUTP. After purification of the labeled cDNA (Qiagen PCR purification kit), cDNA was resuspended in individually prepared Cy3 and Cy5 aliquots as appropriate (GE Healthcare, Quebec, Canada). The reaction was then quenched and unincorporated Cy dyes was removed (Qiagen PCR purification kit). Labeled sample cDNA and reference cDNA was then combined and hybridized onto the micro-arrays. After incubation overnight in hybridization chambers (BioRad, Mississauga, Ontario, Canada) the arrays were washed, dried and the fluorescent signals on the micro-array were imaged and scanned (Axon Genepix 4000A). Subsequently, a ratio of the fluorescence of the two fluors was obtained for each DNA spot on the array. The principal behind this approach was that this ratio was proportional to the relative expression of that RNA species in the tissue sample. Since human universal reference RNA was used as a control for each chip, ratios (relative RNA expression) could be compared across all chips. Statistical algorithms and pattern recognition techniques were then applied to ratio data to identify gene or gene clusters that were specific to experimental endpoints. An outline of the method is illustrated in FIG. 1.

Primary Outcome Measures

The primary outcome measures in the study were relative measures of gene expression compared to gestation at delivery. The four time points for delivery that were considered in the study were: 1) delivery within 48 hours of clinical presentation (Table 1); 2) delivery within 14 days of clinical presentation (Table 2); 3) delivery prior to 34 weeks gestation (Table 3); and 4) delivery prior to 37 weeks gestation (Table 4). The first two time points (delivery within 48 hours and 7 days) were selected as they are clinically relevant for decision making relating to corticosteroid therapy and transfer to tertiary centers for potential delivery. The second two time points (delivery prior to 34 and 37 weeks) were considered to enable a direct comparison with previously published data relating to the predictive value of fetal fibronectin testing which reported predictive values for all four of the time points that were considered in the study.

Of the forty (40) women in the study, twenty-one (21) delivered pre-term (<37 wks) and 19 delivered at term. Of the 21 pre-term births, 12 occurred prior to 34 weeks.

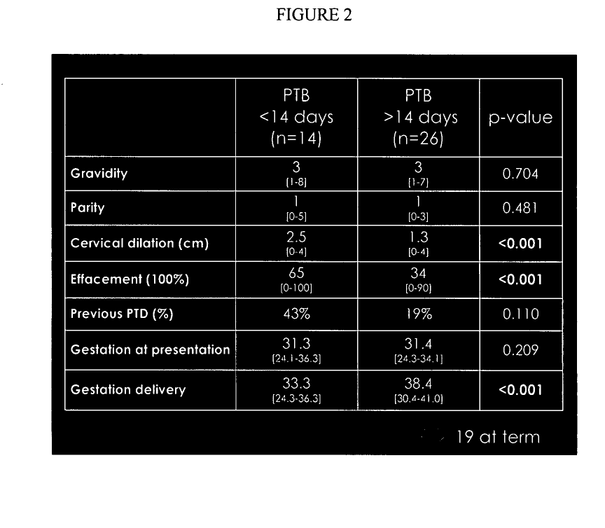

Thirteen (13) of the 21 pre-term births occurred within 48 hours of clinical presentation and 14 occurred within 14 days of clinical presentation. FIG. 2 illustrates the baseline clinical data of the 40 women in the study.

Validation of Micro-Array Data:

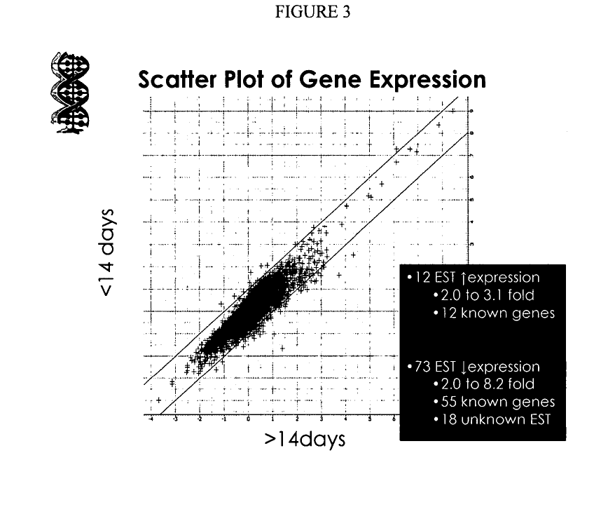

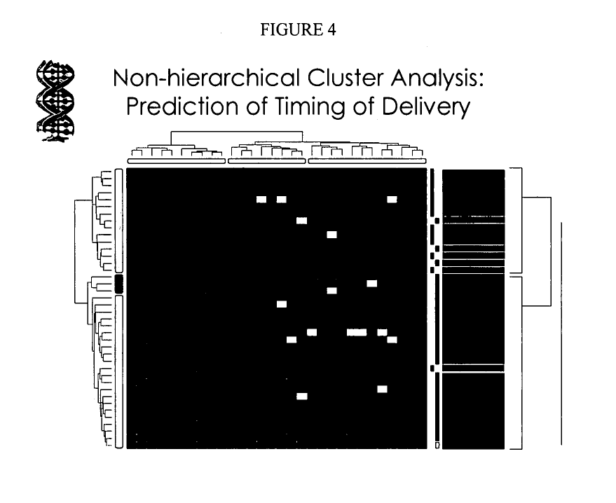

A large number of genes (half up-regulated and half down-regulated) were identified with significantly different gene expression when compared to samples from women who deliver pre-term with samples from women whose pregnancies continue. FIG. 3 is a scatter plot of all 19200 EST on the microarray comparing gene expression in leukocytes between the group of women who progressed to delivery within 14 days and those of delivered greater than 14 days after presentation. The two diagonal lines represent two fold up regulation and down regulation of gene expression comparing the two groups. The red crosses above the diagonal line are 12 ESTs where there was increased expression in women who progressed to delivery. The green crosses below the line represent the 73 ESTs with down regulated gene expression. From this subset of genes, genes could be identified where expression patterns reliably predict the timing of delivery. A number of different techniques can be utilised to identify a set of genes that are capable of predicting a particular outcome, i.e. timing of delivery. FIG. 4 presents one of these techniques—non-hierarchical cluster analysis. On this figure, each gene is represented as a column and each patient is a row of squares. Here 25 genes were filtered out that had the most discordant for delivery in <48 hours. It can be seen from the cluster analysis on the left side of the figure (using non-hierarchical clustering using complete linkage of Euclidean distance) that all bar one case that delivered in less than 48 hours was identified in the upper cluster and the lower cluster identified the vast majority of cases who delivered >48 hours after presentation. This is a lot clearer if the cases are replaced with coloured bars on the right where it can be seen that most of the red is in the upper group and most of the blue is in the lower group. Further, the cluster analysis along the top of the diagram is a cluster analysis of gene expression patterns. The genes fall into 3 specific groups. In one embodiment, this analysis can be used to identify not only true PTB but also the particular types of PTB such as infection mediated PTB vs. stretch mediated PTB, and the like.

To validate the data obtained from the micro-array studies, real time polymerase chain reaction (RT-PCR) was performed on selected genes. The genes selected for RT-PCR confirmation were those with the greatest ability to predict the outcome groups.

Complementary DNA is synthesized from each maternal RNA sample using the TaqMan Reverse Transcription Kit (Applied Biosystems, Foster City, Calif., USA). The total volume of the reverse transcription reaction is 100 μl, which contains 110 μl of 10× TaqMan RT buffer, 22 μl of 25 mM MgCl₂, 201 of 2.5 mM deoxyNTPs mixture, 5 μl of 50 uM random hexamers, 2 μl of 20 U/L RNase inhibitor, 2.5 μl of 50 U/μl MultiScribe Reverse Transcriptase, 26.5 μl of RNase-free water, and 10 μl of 100 mM DTT (Invitrogen, GmbH, Karlsruhe, Germany). To this mixture, 21g of total RNA is added for conversion into cDNA. The thermal cycling conditions include primer incubation step for 10 min at 25° C., reverse transcription for 30 min at 48° C., and reverse transcription inactivation for 5 min at 95° C.

Gene-specific primers are then designed using Primer Express (Applied Biosystems) to fulfill all criteria for real-time PCR primers. PCR is performed in an optical 96-well plate with an ABI PRISM 7900 HT Sequence Detection System (Applied Biosystems), using the SYBR Green detection chemistry. Reaction conditions for each gene including primer and template concentrations, and thermal profile will be optimized. Each reaction contains 12.5 μl of 2× SYBR Green master mix, optimized amount of primers and cDNA concentration, and water to make up a total reaction volume of 25 μl. A general thermal profile is 95° C. for 10 min, 40 cycles of 95° C. for 15 sec and 60° C. for 1 min. After PCR, a dissociation curve is constructed by increasing temperature from 65° C. to 95° C. for detection of PCR product specificity.

Data was analyzed using the SDS 2.1 software (Applied Biosystems). The relative standard curve or the delta Ct method was used for data analysis, depending on the number of genes that display significant expression changes from micro-array. The results are illustrated in Tables 1-4 and 5 and 6.

Development of the Custom Pre-Term Labor Micro-Array

The “pre-term birth genetic signature” determined as described herein will be the basis for the development of a custom pre-term labor micro-array. Custom micro-arrays can be produced using both cDNA and oligonucleotide technology. These are currently being produced by a large number of both commercial and academic institutions.

When developing a custom micro-array to validate a “gene expression signature” for particular conditions it is important that the number of genes represented on the array is not overly restrictive. The subset of genes for the custom array should include: all of the sequences with the highest ability to discriminate outcomes; all of the sequences with significant changes in expression (>2-fold change) identified comparing the experimental groups and; an adequate sample of sequences (approximately one third of the total) where there is no change in expression between the study groups to enable reliable normalization and interpretation of data.

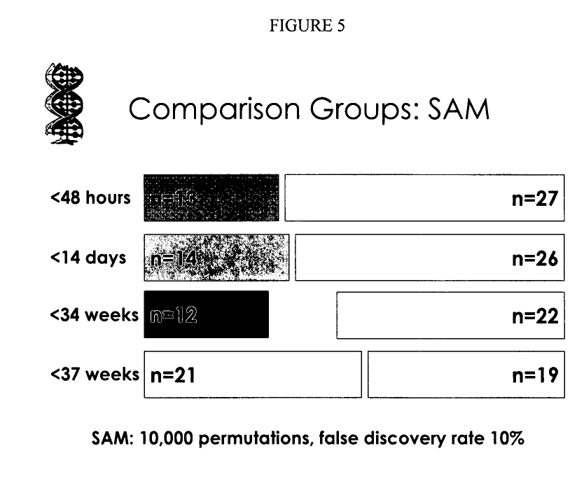

The four specific outcome groups evaluated in this study are presented in FIG. 5. To determine statistically significant differences in gene expression based on micro-array data, and account for the issue of multiple testing, a technique called Statistical Analysis of Microarray data or SAM was used. SAM uses a combination of multiple permutations to identify false positives and allows the definition of a false discovery rate prior to the analyses, to address the issue of multiple testing. For 10,000 permutations, the false discovery rate was set at 10%.

Comparing the four outcome groups, the inventors identified 146 ESTs that were significantly different in the group that delivered less that 48 hours after presentation, 92 that were different in the group delivering in less than 14 days, 19 that were different in the less than 34 week group and 19 that were different in the less than 37 week group. These EST's are associated with 118, 74, 16 and 15 genes respectively. Although there is some overlap between these groups, many of the genes that were different at each time point are specific for that time point—e.g., not all of the 74 genes in the <14 day group are in the 118 genes that were different at 48 hours. As such, in one aspect, the invention further provides the ability to identify different gene expression signatures for delivery at each specific time point. The results are illustrated in Tables 1-4 for the respective groups (Table 1, PTB<48 hours from clinical presentation; Table 2, PTB<14 days from clinical presentation; Table 3, PTB<34 weeks; Table 4 PTB<37 weeks. Group comparisons were performed using SAM (Statistical Analyses of Microarray).

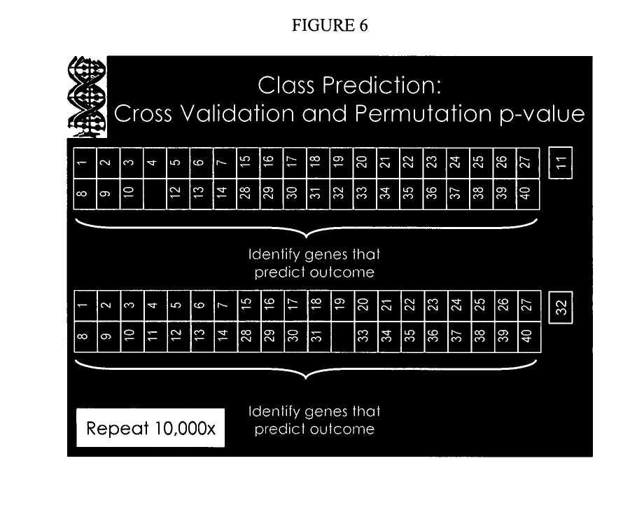

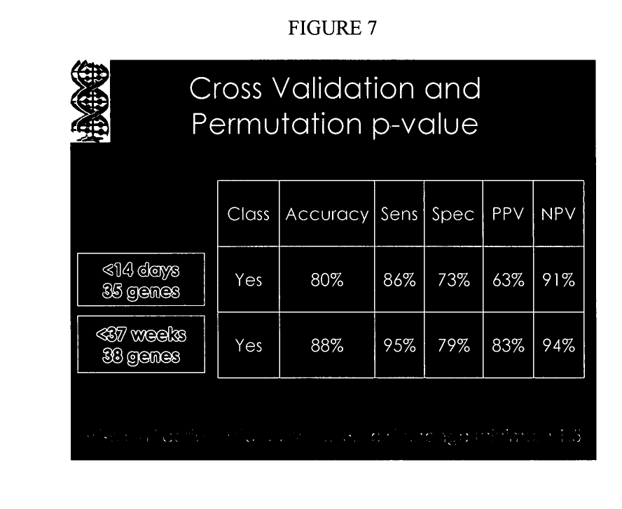

One of the problems with traditional cluster analysis is that predictors developed on a set of patients are effective on that population but not necessarily translatable to different populations. Cross validation and permutation p-value is one of a number of techniques developed to try and get around this problem. In brief, one case is randomly removed from the dataset. A set of predictive genes for a particular outcome is then identified and then tested as a predictor on the excluded case. This is then repeated with a different case randomly excluded—and then this is repeated a number of times—10,000× was used in the present case. See FIG. 6 for illustration of method and FIG. 7 for cross-validation and permutation P-value statistics for the different groups illustrated. The p-values (and t-values) for predictors of pre-term delivery <14 days is illustrated in Table 5. Table 5 illustrates expression sequence tags (ESTs) that were statistically different between women who delivered <14 days compared to those who delivered >14 days. SAM was utilized using a predetermined false discovery rate of 10% and 10,000 permutations. Table 6 illustrates ESTs that were identified as predictors of delivery in the <34 weeks pre-term delivery group. Genes were selected for prediction models based on univariate misclassification rate of less than 0.25 with fold-ratio of geometric means between the two classes exceeding 1.5. Leave-one-out method was used for cross validation of predictors.

The custom pre-term labour micro-arrays can be used to investigate gene-environment interactions in pre-term birth. In addition the custom micro-array will have the ability to potentially discriminate between pre-term labors resulting from different etiologies (e.g. infection vs. fetal stress vs. myometrial activation) based on the differential gene expression profiles that might be induced. This may ultimately provide the opportunity for the provision of an individualized therapeutic strategy that is likely to be a more effective approach to the management of pre-term labor.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the domains, cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a cell” includes a plurality of such cells, reference to the “antibody” is a reference to one or more antibodies and equivalents thereof known to those skilled in the art, and so forth.

Table Legend

In Tables 1-6, Group comparisons were performed using SAM (Statistical Analyses of Microarray) which uses both multiple permutations and a predetermined false discovery rate to identify gene expression that is different between two specific outcomes.

Table 1. Expression sequence tags (ESTs) that were statistically different between women who delivered <48 hours compared to those who delivered >48 hours. SAM was utilised using a predetermined false discovery rate of 10% and 10,000 permutations.

Table 2. Expression sequence tags (ESTs) that were statistically different between women who delivered <14 days compared to those who delivered >14 days. SAM was utilised using a predetermined false discovery rate of 10% and 10,000 permutations.

Table 3. Expression sequence tags (ESTs) that were statistically different between women who delivered <34 weeks gestation compared to those who delivered >34 weeks gestation. SAM was utilised using a predetermined false discovery rate of 10% and 10,000 permutations.

Table 4. Expression sequence tags (ESTs) that were statistically different between women who delivered <37 weeks gestation compared to those who delivered >37 weeks gestation. SAM was utilised using a predetermined false discovery rate of 10% and 10,000 permutations.

Table 5. ESTs that were identified as predictors of delivery in <14 days from clinical presentation. Genes were selected for prediction models based on univariate misclassification rate of less than 0.25 with fold-ratio of geometric means between the two classes exceeding 1.5. Leave-one-out method was used for cross validation of predictors.

Table 6 ESTs that were identified as predictors of delivery in <37 weeks gestation. Genes were selected for prediction models based on univariate misclassification rate of less than 0.25 with fold-ratio of geometric means between the two classes exceeding 1.5. Leave-one-out method was used for cross validation of predictors.

TABLE 1
Primary Outcome Measures: Pre-Term Delivery less than 48 hours from Clinical Presentation

| | Geom | Geom | | | |
|---|---|---|---|---|---|
| | mean of | mean of | Fold | | |
| | ratios in | ratios in | difference | | |
| | class 1: | class 2: | of geom | | |
| | No | Yes | means | Unique id | Description |
| 1 | 0.677 | 0.4 | 1.693 | 327676 | Keratin 13 |
| 2 | 0.644 | 0.431 | 1.494 | 219590 | Chromosome 8 open reading frame 1 |
| 3 | 0.788 | 0.499 | 1.579 | 277523 | Transcribed locus |
| 4 | 0.656 | 0.367 | 1.787 | 40134 | Major facilitator superfamily domain |
| | | | | | containing 3 |
| 5 | 1.072 | 0.807 | 1.328 | 503300 | WD repeat domain 5B |
| 6 | 0.726 | 0.434 | 1.673 | 273048 | Muscleblind-like 2 (Drosophila) |
| 7 | 0.753 | 0.42 | 1.793 | 152863 | Phospholipid transfer protein |
| 8 | 0.784 | 0.553 | 1.418 | 214113 | Transmembrane channel-like 1 |
| 9 | 0.703 | 0.482 | 1.459 | 223589 | Leukocyte cell derived chemotaxin 1 |
| 10 | 0.539 | 0.307 | 1.756 | 32221 | Rho GDP dissociation inhibitor (GDI) |
| | | | | | alpha |
| 11 | 0.818 | 0.548 | 1.493 | 273492 | Chromosome 14 open reading frame 142 |
| 12 | 0.979 | 0.729 | 1.343 | 342827 | Hypothetical protein MGC3196 |
| 13 | 0.835 | 0.572 | 1.46 | 469201 | Hormonally upregulated New-associated |
| | | | | | kinase |
| 14 | 0.902 | 0.543 | 1.661 | 471129 | UPF3 regulator of nonsense transcripts |
| | | | | | homolog A (yeast) |
| 15 | 0.812 | 0.579 | 1.402 | 485952 | CCR4-NOT transcription complex, |
| | | | | | subunit 8 |
| 16 | 0.589 | 0.348 | 1.693 | 222563 | Oxidase assembly 1-like, mRNA (cDNA |
| | | | | | clone MGC: 28641 IMAGE: 4223847) |
| 17 | 0.97 | 0.716 | 1.355 | 270300 | Transgelin |
| 18 | 0.693 | 0.509 | 1.361 | 214169 | |
| 19 | 0.69 | 0.382 | 1.806 | 151515 | Hypothetical protein MGC26963 |
| 20 | 0.997 | 0.668 | 1.493 | 210551 | Tumor rejection antigen (gp96) 1 |
| 21 | 0.874 | 0.609 | 1.435 | 282302 | |
| 22 | 0.488 | 0.282 | 1.73 | 213747 | Myosin IB |
| 23 | 0.775 | 0.57 | 1.36 | 109858 | Cytoplasmic linker associated protein 1 |
| 24 | 0.701 | 0.509 | 1.377 | 215003 | UDP-Gal:betaGlcNAc beta 1,4- |
| | | | | | galactosyltransferase, polypeptide 1 |
| 25 | 1.258 | 0.877 | 1.434 | 503345 | Fatty acid binding protein 4, adipocyte |
| 26 | 0.929 | 0.579 | 1.604 | 486465 | Transcribed locus |
| 27 | 0.853 | 0.558 | 1.529 | 471734 | Glutamate-rich 1 |
| 28 | 0.722 | 0.497 | 1.453 | 471889 | Nuclear receptor subfamily 2, group F, |
| | | | | | member 1 |
| 29 | 0.815 | 0.517 | 1.576 | 40635 | Prickle-like 1 (Drosophila) |
| 30 | 0.822 | 0.563 | 1.46 | 145731 | SEC 15-like 2 (S. cerevisiae) |
| 31 | 0.784 | 0.57 | 1.375 | 172807 | Glutamate receptor, ionotrophic, AMPA 4 |
| 32 | 0.915 | 0.633 | 1.445 | 340511 | Adipose differentiation-related protein |
| 33 | 0.698 | 0.448 | 1.558 | 502362 | BCL2-associated athanogene 3 |
| 34 | 0.925 | 0.607 | 1.524 | 282099 | |
| 35 | 0.662 | 0.359 | 1.844 | 47178 | Chromosome 21 open reading frame 42 |
| 36 | 0.862 | 0.576 | 1.497 | 307297 | Peptidylprolyl isomerase domain and WD |
| | | | | | repeat containing 1 |
| 37 | 0.982 | 0.637 | 1.542 | 376845 | ATP-binding cassette, sub-family A |
| | | | | | (ABC1), member 9 |
| 38 | 0.628 | 0.388 | 1.619 | 154170 | Protein tyrosine phosphatase, non- |
| | | | | | receptor type 23 |
| 39 | 0.766 | 0.543 | 1.411 | 239886 | |
| 40 | 0.91 | 0.666 | 1.366 | 485736 | Poly (ADP-ribose) polymerase family, |
| | | | | | member 8 |
| 41 | 0.572 | 0.36 | 1.589 | 206793 | Kelch domain containing 3 |
| 42 | 0.665 | 0.429 | 1.55 | 273602 | PiggyBac transposable element derived 2 |
| 43 | 0.858 | 0.631 | 1.36 | 683306 | Ribosomal protein S2 |
| 44 | 1.056 | 0.722 | 1.463 | 294161 | Ankyrin repeat domain 10 |
| 45 | 0.859 | 0.614 | 1.399 | 261713 | Protein tyrosine phosphatase, receptor |
| | | | | | type, M |
| 46 | 0.859 | 0.618 | 1.39 | 428713 | Importin 9 |
| 47 | 0.745 | 0.54 | 1.38 | 503743 | Pregnancy-associated plasma protein A, |
| | | | | | pappalysin 1 |
| 48 | 0.928 | 0.641 | 1.448 | 504173 | SWI/SNF related, matrix associated, |
| | | | | | actin dependent regulator of chromatin, |
| | | | | | subfamily c, member 2 |
| 49 | 1.183 | 0.861 | 1.374 | 280586 | 60S ribosomal protein L6 (RPL6A) |
| 50 | 0.725 | 0.479 | 1.514 | 327677 | Periostin, osteoblast specific factor |
| 51 | 0.936 | 0.578 | 1.619 | 229486 | Chromosome 14 open reading frame 106 |
| 52 | 0.89 | 0.641 | 1.388 | 487825 | FCH and double SH3 domains 1 |
| 53 | 0.858 | 0.595 | 1.442 | 299067 | Phosphatidylinositol glycan, class K |
| 54 | 0.872 | 0.629 | 1.386 | 485944 | Matrix metallopeptidase 11 (stromelysin |
| | | | | | 3) |
| 55 | 0.866 | 0.629 | 1.377 | 484939 | Transcribed locus |
| 56 | 1.026 | 1.807 | 0.568 | 47408 | GrpE-like 2, mitochondrial (E. coli) |
| 57 | 0.991 | 1.665 | 0.595 | 195917 | Family with sequence similarity 20, |
| | | | | | member C |
| 58 | 1.694 | 2.772 | 0.611 | 683190 | Transcribed locus, weakly similar to |
| | | | | | XP_512541.1 PREDICTED: similar to |
| | | | | | hypothetical protein [Pan troglodytes] |
| 59 | 0.854 | 1.343 | 0.636 | 323823 | Zinc finger protein 364 |
| 60 | 1.714 | 2.676 | 0.641 | 167085 | Synaptotagmin XI |
| 61 | 1.044 | 1.789 | 0.584 | 183230 | FK506 binding protein 14, 22 kDa |
| 62 | 1.721 | 2.838 | 0.606 | 193713 | Torsin A interacting protein 1 |
| 63 | 0.881 | 1.348 | 0.654 | 42910 | Malic enzyme 1, NADP(+)-dependent, |
| | | | | | cytosolic |
| 64 | 1.331 | 1.978 | 0.673 | 203767 | |
| 65 | 0.911 | 1.474 | 0.618 | 37458 | Hydroxysteroid (17-beta) dehydrogenase 6 |
| 66 | 1.127 | 1.98 | 0.569 | 238885 | |
| 67 | 1.602 | 2.724 | 0.588 | 71211 | |
| 68 | 1.019 | 1.656 | 0.615 | 29947 | Zic family member 4 |
| 69 | 0.784 | 1.353 | 0.579 | 43158 | Hypothetical protein FLJ21908 |
| 70 | 0.706 | 1.435 | 0.492 | 29331 | TruB pseudouridine (psi) synthase |
| | | | | | homolog 1 (E. coli) |
| 71 | 1.1 | 1.749 | 0.629 | 119625 | Activating signal cointegrator 1 complex |
| | | | | | subunit 3-like 1 |
| 72 | 0.83 | 1.877 | 0.442 | 200260 | Cytochrome P450, family 3, subfamily A, |
| | | | | | polypeptide 7 |
| 73 | 0.766 | 1.501 | 0.51 | 32102 | Transcribed locus |
| 74 | 1.152 | 1.78 | 0.647 | 270849 | Jumonji, AT rich interactive domain 2 |
| 75 | 0.924 | 1.472 | 0.628 | 166141 | Mannosyl (alpha-1,6-)-glycoprotein beta- |
| | | | | | 1,6-N-acetyl-glucosaminyltransferase, |
| | | | | | isoenzyme B |
| 76 | 0.728 | 1.24 | 0.587 | 151231 | C-type lectin domain family 9, member A |
| 77 | 1.368 | 2.017 | 0.678 | 198559 | KIAA0853 |
| 78 | 1.283 | 2.174 | 0.59 | 346396 | Homeodomain interacting protein kinase 2 |
| 79 | 0.847 | 1.315 | 0.644 | 33743 | |
| 80 | 1.519 | 2.14 | 0.71 | 491648 | Reticulocalbin 1, EF-hand calcium |
| | | | | | binding domain |
| 81 | 1.171 | 1.935 | 0.605 | 186852 | Ribosomal protein S6 kinase, 90 kDa, |
| | | | | | polypeptide 2 |
| 82 | 1.105 | 1.738 | 0.636 | 186842 | Pericentriolar material 1 |
| 83 | 1.264 | 1.925 | 0.657 | 182864 | Uveal autoantigen with coiled-coil |
| | | | | | domains and ankyrin repeats |
| 84 | 0.806 | 1.132 | 0.712 | 41803 | Splicing factor, arginine/serine-rich 14 |
| 85 | 1.311 | 1.853 | 0.708 | 253268 | SDS3 protein |
| 86 | 0.89 | 1.19 | 0.748 | 40300 | SYF2 homolog, RNA splicing factor (S. cerevisiae) |
| 87 | 1.349 | 1.901 | 0.71 | 195513 | Alanine-glyoxylate aminotransferase 2 |
| 88 | 0.854 | 1.164 | 0.734 | 199177 | Glucosidase, alpha; neutral C |
| 89 | 1.115 | 1.704 | 0.654 | 119429 | Ring finger protein 10 |
| 90 | 0.527 | 1.053 | 0.5 | 27870 | Mesenchyme homeo box 2 (growth arrest |
| | | | | | specific homeo box) |
| 91 | 0.87 | 1.412 | 0.616 | 31893 | Family with sequence similarity 11, |
| | | | | | member A |
| 92 | 1.188 | 1.843 | 0.645 | 186831 | Zinc finger protein 32 (KOX 30) |
| 93 | 1.086 | 1.395 | 0.778 | 177057 | Hypothetical protein LOC149351 |
| 94 | 0.796 | 1.479 | 0.538 | 27217 | Transcribed locus |
| 95 | 1.043 | 1.687 | 0.618 | 66380 | Internexin neuronal intermediate filament |
| | | | | | protein, alpha |
| 96 | 0.891 | 1.539 | 0.579 | 28361 | Ras-related GTP binding D |
| 97 | 1.122 | 1.534 | 0.731 | 67002 | |
| 98 | 0.852 | 1.231 | 0.692 | 39167 | Acyl-Coenzyme A oxidase 3, pristanoyl |
| 99 | 0.953 | 1.274 | 0.748 | 418296 | |
| 100 | 0.681 | 1.229 | 0.554 | 39153 | Cullin-assodated and neddylation- |
| | | | | | dissociated 1 |
| 101 | 0.955 | 1.335 | 0.723 | 45921 | Hypothetical protein MGC11332 |
| 102 | 0.905 | 1.264 | 0.716 | 179846 | KIAA0773 gene product |
| 103 | 1.109 | 1.656 | 0.67 | 151941 | 26 serine protease |
| 104 | 1.159 | 1.565 | 0.741 | 504180 | Hypothetical gene supported by |
| | | | | | AK026189 |
| 105 | 1.148 | 1.636 | 0.702 | 376888 | S100 calcium binding protein, beta |
| | | | | | (neural) |
| 106 | 1.256 | 1.943 | 0.646 | 182717 | Valyl-tRNA synthetase |
| 107 | 1.002 | 1.742 | 0.575 | 126592 | COBL-like 1 |
| 108 | 0.658 | 1.011 | 0.651 | 26863 | TBC1 domain family, member 8 (with |
| | | | | | GRAM domain) |
| 109 | 0.701 | 1.069 | 0.656 | 347751 | Gardner-Rasheed feline sarcoma viral (v- |
| | | | | | fgr) oncogene homolog |
| 110 | 0.908 | 1.405 | 0.646 | 50990 | Family with sequence similarity 13, |
| | | | | | member C1 |
| 111 | 0.948 | 1.452 | 0.653 | 42439 | |
| 112 | 1.058 | 1.545 | 0.685 | 250092 | Heterogeneous nuclear ribonucleoprotein F |
| 113 | 0.552 | 1.044 | 0.529 | 22896 | ADP-dependent glucokinase |
| 114 | 2.522 | 3.662 | 0.689 | 195110 | Hypothetical protein LOC339751 |
| 115 | 1.094 | 1.534 | 0.713 | 195875 | Chromosome 18 open reading frame 1 |
| 116 | 0.861 | 1.617 | 0.532 | 32305 | Ets variant gene 1 |
| 117 | 0.698 | 1.378 | 0.507 | 30262 | Coiled-coil domain containing 14 |
| 118 | 1.27 | 1.713 | 0.741 | 239288 | Transcribed locus |
| 119 | 1.172 | 1.718 | 0.682 | 152377 | GTPase activating Rap/RanGAP domain- |
| | | | | | like 3 |
| 120 | 0.876 | 1.267 | 0.691 | 151605 | |
| 121 | 0.95 | 1.335 | 0.712 | 42081 | Hypothetical protein MGC5242 |
| 122 | 0.875 | 1.226 | 0.714 | 51606 | Hypothetical LOC389182 |
| 123 | 0.719 | 1.13 | 0.636 | 26823 | ATG5 autophagy related 5 homolog S. cerevisiae) |
| 124 | 1.018 | 1.542 | 0.66 | 377166 | Calsequestrin 2 (cardiac muscle) |
| 125 | 1.117 | 1.685 | 0.663 | 120006 | CDNA FLJ44280 fis. clone |
| | | | | | TRACH2001684 |
| 126 | 0.848 | 1.193 | 0.711 | 39191 | KIAA1912 protein |
| 127 | 0.923 | 1.278 | 0.722 | 33196 | Similar to BcDNA: GH11415 gene product |
| 128 | 1.156 | 1.522 | 0.76 | 239874 | Transcribed locus |
| 129 | 1.561 | 2.483 | 0.629 | 252993 | CDNA FLJ46752 fis, clone |
| | | | | | TRACH3023063 |
| 130 | 1.421 | 2.044 | 0.695 | 177788 | Zinc finger protein, multitype 2 |
| 131 | 0.932 | 1.235 | 0.755 | 37455 | PR domain containing 10 |
| 132 | 0.921 | 1.347 | 0.684 | 28147 | Megalencephalic leukoencephalopathy |
| | | | | | with subcortical cysts 1 |
| 133 | 1.224 | 2.06 | 0.594 | 665661 | Hypothetical protein MGC29671 |
| 134 | 1.085 | 1.619 | 0.67 | 37371 | RAB9B, member RAS oncogene family |
| 135 | 0.761 | 1.105 | 0.689 | 27687 | Transcribed locus |
| 136 | 1.017 | 1.472 | 0.691 | 29988 | |
| 137 | 0.635 | 0.994 | 0.639 | 27210 | Catenin (cadherin-associated protein), |
| | | | | | alpha 2 |
| 138 | 0.882 | 1.608 | 0.549 | 192364 | Clone 24841 mRNA sequence |
| 139 | 1.722 | 2.812 | 0.612 | 286320 | Eukaryotic translation initiation factor 2, |
| | | | | | subunit 3 gamma, 52 kDa |
| 140 | 0.899 | 1.169 | 0.769 | 110747 | Chemokine-like factor |
| 141 | 1.124 | 1.775 | 0.633 | 683068 | Hypothetical protein MGC52498 |
| 142 | 0.984 | 1.383 | 0.711 | 32103 | Hypothetical protein FLJ11506 |
| 143 | 0.975 | 1.356 | 0.719 | 42819 | Numb homolog (Drosophila) |
| 144 | 1.029 | 1.459 | 0.705 | 26588 | Similar to CG11994-PA |
| 145 | 0.907 | 1.347 | 0.673 | 30103 | MRNA full length insert cDNA clone |
| | | | | | EUROIMAGE 30103 |
| 146 | 0.966 | 1.379 | 0.701 | 34407 | Hypothetical protein LOC283267 |

| | Clone | GB acc | UG cluster | Gene symbol | Map Location |
|---|---|---|---|---|---|
| 1 | 327676 | W35201 | Hs.463032 | KRT13 | 17q12-q21.2 |
| 2 | 219590 | H83804 | Hs.436445 | C8orf1 | 8q21 |
| 3 | 277523 | N47352 | Hs.445365 | | |
| 4 | 40134 | R53347 | Hs.7678 | LOC113655 | 8q24.3 |
| 5 | 501516 | AA135682 | Hs.142395 | WDR5B | 3q21.1 |
| 6 | 273048 | N44278 | Hs.125715 | MBNL2 | 13q32.1 |
| 7 | 152863 | | Hs.439312 | PLTP | 20q12-q13.1 |
| 8 | 214113 | H72508 | Hs.371614 | TMC1 | 9q21.12 |
| 9 | 223589 | H86896 | Hs.421391 | LECT1 | 13q14-q21 |
| 10 | 5180471 | BI821973 | Hs.159161 | ARHGDIA | 17q25.3 |
| 11 | 5804008 | BQ054704 | Hs.20142 | C14orf142 | 14q32.13 |
| 12 | 342827 | | Hs.381134 | LOC374395 | 11q12.3 |
| 13 | 469201 | | Hs.109437 | HUNK | 21q22.1 |
| 14 | 471129 | AA034360 | Hs.533855 | UPF3A | 13q34 |
| 15 | 488564 | AA044787 | Hs.26703 | CNOT8 | 5q31-q33 |
| 16 | 5702324 | BM994858 | Mm.182340 | Oxa1I | 14 C1 |
| 17 | 270300 | BM852954 | Hs.503998 | TAGLN | 11q23.2 |
| 18 | 214169 | | Data not | | |
| | | | found | | |
| 19 | 151515 | H03751 | Hs.48343 | MGC26963 | 4q25 |
| 20 | 210551 | | Hs.192374 | TRA1 | 12q24.2-q24.3 |
| 21 | 282302 | N53743 | Hs.257341 | | |
| 22 | 213747 | H71756 | Hs.439620 | MYO1B | 2q12-q34 |
| 23 | 109858 | T84239 | Hs.469840 | CLASP1 | 2q14.2-q14.3 |
| 24 | 215003 | H74175 | Hs.272011 | B4GALT1 | 9p13 |
| 25 | 503345 | AA128249 | Hs.391561 | FABP4 | 8q21 |
| 26 | 486465 | BM686432 | Hs.553244 | | |
| 27 | 471734 | AA035475 | Hs.128130 | LOC157697 | 8p23.3 |
| 28 | 471889 | AA035764 | Hs.519445 | NR2F1 | 5q14 |
| 29 | 40635 | | Hs.524348 | PRICKLE1 | 12q12 |
| 30 | 145731 | R78322 | Hs.303454 | SEC15L2 | 2p13.2 |
| 31 | 172807 | H19718 | Hs.503743 | GRIA4 | 11q22 |
| 32 | 340511 | W55903 | Hs.3416 | ADFP | 9p22.1 |
| 33 | 5541293 | BM800951 | Hs.523309 | BAG3 | 10q25.2-q26.2 |
| 34 | 282074 | N51481 | Data not | | |
| | | | found | | |
| 35 | 3223091 | BE672389 | Hs.234016 | C21orf42 | 21q21.3 |
| 36 | 307297 | W21229 | Hs.121432 | KIAA0073 | 5q12.3 |
| 37 | 1857635 | AI284184 | Hs.131686 | ABCA9 | 17q24.2 |
| 38 | 154170 | R52028 | Hs.25524 | PTPN23 | 3p21.3 |
| 39 | 239886 | Unresolved | Data not | | |
| | | | found | | |
| 40 | 485736 | Unresolved | Hs.369581 | PARP8 | 5q11.1 |
| 41 | 206793 | R98288 | Hs.412468 | KLHDC3 | 6p21.1 |
| 42 | 273602 | N46282 | Hs.433322 | PGBD2 | 1q44 |
| 43 | 683306 | | Hs.506997 | RPS2 | 16p13.3 |
| 44 | 294161 | W01990 | Hs.525163 | ANKRD10 | 13q34 |
| 45 | 261713 | Unresolved | Hs.49774 | PTPRM | 18p11.2 |
| 46 | 428713 | Unresolved | Hs.497384 | IPO9 | 1q32.1 |
| 47 | 503743 | AA131649 | Hs.494928 | PAPPA | 9q33.2 |
| 48 | 504173 | AA132128 | Hs.236030 | SMARCC2 | 12q13-q14 |
| 49 | 280586 | N50403 | Hs.524599 | NAP1L1 | 12q21.2 |
| 50 | 327677 | W35228 | Hs.136348 | POSTN | 13q13.3 |
| 51 | 229486 | Unresolved | Hs.437941 | C14orf106 | 14q21.3 |
| 52 | 446626 | AA203283 | Hs.120094 | FCHSD1 | 5q31.3 |
| 53 | 4806339 | BG778094 | Hs.293653 | PIGK | 1p31.1 |
| 54 | 485944 | AA040502 | Hs.143751 | MMP11 | 22q11.2 |
| 55 | 484939 | AA037633 | Hs.191422 | | |
| 56 | 47408 | H10463 | Hs.511816 | GRPEL2 | 5q32 |
| 57 | 195917 | R92625 | Hs.512235 | ITPR2 | 12p11 |
| 58 | 683190 | | Hs.536673 | | |
| 59 | 323823 | W46196 | Hs.523550 | ZNF364 | 1q21.1 |
| 60 | 167085 | R89738 | Hs.32984 | SYT11 | 1q21.2 |
| 61 | 183230 | H45017 | Hs.390838 | FKBP14 | 7p15.1 |
| 62 | 193713 | H47769 | Hs.496459 | LAP1B | 1q24.2 |
| 63 | 42910 | R60083 | Hs.21160 | ME1 | 6q12 |
| 64 | 203767 | H56293 | Data not | | |
| | | | found | | |
| 65 | 37458 | R35197 | Hs.524513 | RODH | 12q13 |
| 66 | 238885 | | Data not | | |
| | | | found | | |
| 67 | 71211 | | Data not | | |
| | | | found | | |
| 68 | 29947 | | Hs.415766 | ZIC4 | 3q24 |
| 69 | 43158 | R60476 | Hs.437855 | FLJ21908 | 12q13.11 |
| 70 | 29331 | R12880 | Hs.21187 | TRUB1 | 10q25.3 |
| 71 | 119625 | | Hs.246112 | ASCC3L1 | 2q11.2 |
| 72 | 200260 | R96774 | Hs.111944 | CYP3A7 | 7q21-q22.1 |
| 73 | 49443 | H15482 | Hs.22930 | | |
| 74 | 270849 | N42634 | Hs.269059 | JARID2 | 6p24-p23 |
| 75 | 166141 | R87580 | Hs.144531 | MGAT5B | 17q25.2 |
| 76 | 151231 | H02425 | Hs.531189 | UNQ9341 | 12p13.31-p13.2 |
| 77 | 198559 | R94802 | Hs.136102 | KIAA0853 | 13q14.13 |
| 78 | 4933116 | BG820204 | Hs.397465 | HIPK2 | 7q32-q34 |
| 79 | 31060 | R42493 | Data not | | |
| | | | found | | |
| 80 | 491648 | BM969982 | Hs.97887 | RCN1 | 11p13 |
| 81 | 186852 | R88127 | Hs.147119 | RPS6KA2 | 6q27 |
| 82 | 186842 | R88122 | Hs.491148 | PCM1 | 8p22-p21.3 |
| 83 | 182864 | H45100 | Hs.33032 | UACA | 15q22-q24 |
| 84 | 41803 | R59287 | Hs.515271 | SFRS14 | 19p12 |
| 85 | 3573662 | BF431222 | Hs.416630 | SDS3 | 12q24.23 |
| 86 | 5933194 | BQ052067 | Hs.20013 | P29 | 1p36.11 |
| 87 | 195513 | R91749 | Hs.34494 | AGXT2 | 5p13 |
| 88 | 199177 | R95789 | Hs.143261 | CAPN3 | 15q15.1-q21.1 |
| 89 | 119429 | T94904 | Hs.442798 | RNF10 | 12q24.31 |
| 90 | 27870 | R13205 | Hs.510504 | MEOX2 | 7p22.1-p21.3 |
| 91 | 31893 | R17865 | Hs.522172 | FAM11A | Xq28 |
| 92 | 186831 | R88121 | Hs.522885 | ZNF32 | 10q22-q25 |
| 93 | 177057 | H40961 | Hs.546492 | LOC149351 | 1p22.2 |
| 94 | 27217 | | Hs.410148 | | |
| 95 | 66380 | T66888 | Hs.500916 | INA | 10q24.33 |
| 96 | 28361 | R13372 | Hs.485938 | RRAGD | 6q15-q16 |
| 97 | 67002 | T70366 | Data not | | |
| | | | found | | |
| 98 | 39167 | R54351 | Hs.479122 | ACOX3 | 4p15.3 |
| 99 | 418296 | W90797 | Hs.26232 | | |
| 100 | 39153 | R54235 | Hs.546407 | TIP120A | 12q14 |
| 101 | 45921 | H08971 | Hs.98798 | MGC11332 | 2q12.1 |
| 102 | 179846 | H51953 | Hs.135343 | KIAA0773 | 7q34 |
| 103 | 4776061 | BG740239 | Hs.997 | P11 | 12q13.1 |
| 104 | 504180 | AA132039 | Hs.399719 | | 6p22.3 |
| 105 | 376888 | | Hs.422181 | S100B | 21q22.3 |
| 106 | 182717 | H43482 | Hs.520026 | VARS2 | 6p21.3 |
| 107 | 126592 | R06875 | Hs.470457 | COBLL1 | 2q24.3 |
| 108 | 26863 | R13756 | Hs.442657 | TBC1D8 | 2q11.2 |
| 109 | 347751 | W81591 | Hs.1422 | FGR | 1p36.2-p36.1 |
| 110 | 50990 | H18544 | Hs.499705 | FAM13C1 | 10q21.1 |
| 111 | 42439 | R61106 | Hs.20107 | | |
| 112 | 250092 | N23517 | Hs.808 | HNRPF | 10q11.21-q11.22 |
| 113 | 22896 | T75252 | Hs.513013 | ADPGK | 15q24.1 |
| 114 | 195110 | R91202 | Hs.444451 | ZAK | 2q24.2 |
| 115 | 195875 | R92320 | Hs.149353 | C18orf1 | 18p11.2 |
| 116 | 39786 | R53267 | Hs.22634 | ETV1 | 7p22 |
| 117 | 4560236 | BG323782 | Hs.17731 | FLJ12892 | 3q21.1 |
| 118 | 239288 | H71486 | Hs.135233 | | |
| 119 | 152377 | R46443 | Hs.29304 | GARNL3 | 9q33.3 |
| 120 | 151605 | H03263 | Hs.12489 | | |
| 121 | 42081 | R61019 | Hs.555958 | MGC5242 | 7q33 |
| 122 | 51606 | H19377 | Hs.22347 | | 3q27.1 |
| 123 | 26823 | R14042 | Hs.486063 | APG5L | 6q21 |
| 124 | 377166 | AA055268 | Hs.57975 | CASQ2 | 1p13.3-p11 |
| 125 | 120006 | T94951 | Hs.209587 | | |
| 126 | 39191 | R54542 | Hs.117136 | KIAA1912 | 2p16.1 |
| 127 | 33196 | R18950 | Hs.151443 | LOC151963 | 3q28-q29 |
| 128 | 239874 | H79772 | Hs.40061 | | |
| 129 | 252993 | H88581 | Hs.370024 | | |
| 130 | 177788 | H46129 | Hs.431009 | ZFPM2 | 8q23 |
| 131 | 37455 | R35195 | Hs.275086 | PRDM10 | 11q25 |
| 132 | 28147 | Unresolved | Hs.517729 | MLC1 | 22q13.33 |
| 133 | 665661 | | Hs.511912 | MGC29671 | 17p13.2 |
| 134 | 37371 | Unresolved | Hs.522736 | RAB9B | Xq22.1-q22.3 |
| 135 | 27687 | R13012 | Hs.412408 | | |
| 136 | | | | | |
| 137 | 5262145 | BI553762 | Hs.167368 | CTNNA2 | 2p12-p11.1 |
| 138 | 192364 | H39125 | Hs.4892 | | |
| 139 | 3432142 | BG055080 | Hs.539684 | EIF2S3 | Xp22.2-p22.1 |
| 140 | 5298687 | T83106 | Hs.15159 | CKLF | 16q22.1 |
| 141 | 683068 | | Hs.424589 | MGC52498 | 1p32.3 |
| 142 | 4779048 | BG741203 | Hs.254642 | FLJ11506 | 15q22.33-q23 |
| 143 | 42819 | R60109 | Hs.509909 | NUMB | 14q24.3 |
| 144 | 26588 | R13891 | Hs.533913 | | 15q15.3 |
| 145 | 30103 | R16307 | Hs.21754 | | |
| 146 | 34407 | R24559 | Hs.44402 | LOC283267 | 11p13 |

TABLE 2
Primary Outcome Measures: Pre-Term Delivery Less Than 14 Days From Clinical Presentation

| | Geom | Geom | | | | | | | | |
|---|---|---|---|---|---|---|---|---|---|---|
| | mean of | mean of | Fold | | | | | | | |
| | ratios in | ratios in | difference | | | | | | | |
| | class 1: | class 2: | of geom | Unique | | | | | | Map |
| | No | Yes | means | id | Description | Clone | GB acc | UG cluster | Gene symbol | Location |
| 1 | 0.689 | 0.407 | 1.693 | 327676 | Keratin 13 | 327676 | W35201 | Hs.463032 | KRT13 | 17q12-q21.2 |
| 2 | 0.647 | 0.444 | 1.457 | 219590 | Chromosome 8 open reading | 219590 | H83804 | Hs.436445 | C8orf1 | 8q21 |
| | | | | | frame 1 | | | | | |
| 3 | 0.659 | 0.384 | 1.716 | 40134 | Major facilitator superfamily | 40134 | R53347 | Hs.7678 | LOC113655 | 8q24.3 |
| | | | | | domain containing 3 | | | | | |
| 4 | 0.788 | 0.573 | 1.375 | 109858 | Cytoplasmic linker associated | 109858 | T84239 | Hs.469840 | CLASP1 | 2q14.2-q14.3 |
| | | | | | protein 1 | | | | | |
| 5 | 0.501 | 0.284 | 1.764 | 213747 | Myosin IB | 213747 | H71756 | Hs.439620 | MYO1B | 2q12-q34 |
| 6 | 0.785 | 0.528 | 1.487 | 277523 | Transcribed locus | 277523 | N47352 | Hs.445365 | | |
| 7 | 0.541 | 0.319 | 1.696 | 32221 | Rho GDP dissociation inhibitor | 5180471 | BI821973 | Hs.159161 | ARHGDIA | 17q25.3 |
| | | | | | (GDI) alpha | | | | | |
| 8 | 0.752 | 0.438 | 1.717 | 152863 | Phospholipid transfer protein | 152863 | | Hs.439312 | PLTP | 20q12-q13.1 |
| 9 | 0.854 | 1.343 | 0.636 | 323823 | Zinc finger protein 364 | 323823 | W46196 | Hs.523550 | ZNF364 | 1q21.1 |
| 10 | 0.981 | 1.608 | 0.61 | 195917 | Family with sequence similarity | 195917 | R92625 | Hs.512235 | ITPR2 | 12p11 |
| | | | | | 20, member C | | | | | |
| 11 | 1.021 | 1.712 | 0.596 | 47408 | GrpE-like 2, mitochondrial (E. coli) | 47408 | H10463 | Hs.511816 | GRPEL2 | 5q32 |
| 12 | 1.703 | 2.762 | 0.617 | 193713 | Torsin A interacting protein 1 | 193713 | H47769 | Hs.496459 | LAP1B | 1q24.2 |
| 13 | 0.899 | 1.445 | 0.622 | 37458 | Hydroxysteroid (17-beta) | 37458 | R35197 | Hs.524513 | RODH | 12q13 |
| | | | | | dehydrogenase 6 | | | | | |
| 14 | 1.004 | 1.637 | 0.613 | 29947 | Zic family member 4 | 29947 | | Hs.415766 | ZIC4 | 3q24 |
| 15 | 1.706 | 2.569 | 0.664 | 167085 | Synaptotagmin XI | 167085 | R89738 | Hs.32984 | SYT11 | 1q21.2 |
| 16 | 1.319 | 1.922 | 0.686 | 203767 | | 203767 | H56293 | Data not | | |
| | | | | | | | | found | | |
| 17 | 1.041 | 1.696 | 0.614 | 183230 | FK506 binding protein 14, 22 kDa | 183230 | H45017 | Hs.390838 | FKBP14 | 7p15.1 |
| 18 | 0.682 | 1.404 | 0.486 | 29331 | TruB pseudauridine (psi) | 29331 | R12880 | Hs.21187 | TRUB1 | 10q25.3 |
| | | | | | synthase homolog 1 (E. coli) | | | | | |
| 19 | 0.901 | 1.465 | 0.615 | 166141 | Mannosyl (alpha-1,6-)- | 166141 | R87580 | Hs.144531 | MGAT5B | 17q25.2 |
| | | | | | glycoprotein beta-1,6-N-acetyl- | | | | | |
| | | | | | glucosaminyltransferase, | | | | | |
| | | | | | isoenzyme B | | | | | |
| 20 | 1.114 | 1.916 | 0.581 | 238885 | | 238885 | | Data not | | |
| | | | | | | | | found | | |
| 21 | 0.877 | 1.294 | 0.678 | 42910 | Malic enzyme 1, NADP(+)- | 42910 | R60083 | Hs.21160 | ME1 | 6q12 |
| | | | | | dependent, cytosolic | | | | | |
| 22 | 0.925 | 1.36 | 0.68 | 42081 | Hypothetical protein MGC5242 | 42081 | R61019 | Hs.555958 | MGC5242 | 7q33 |
| 23 | 0.834 | 1.296 | 0.644 | 33743 | | 31060 | R42493 | Data not | | |
| | | | | | | | | found | | |
| 24 | 1.502 | 2.119 | 0.709 | 491648 | Reticulocalbin 1, EF-hand | 491648 | BM969982 | Hs.97887 | RCN1 | 11p13 |
| | | | | | calcium binding domain | | | | | |
| 25 | 0.78 | 1.286 | 0.607 | 43158 | Hypothetical protein FLJ21908 | 43158 | R60476 | Hs.437855 | FLJ21908 | 12q13.11 |
| 26 | 1.091 | 1.708 | 0.639 | 186842 | Pericentriolar material 1 | 186842 | R88122 | Hs.491148 | PCM1 | 8p22-p21.3 |
| 27 | 1.093 | 1.682 | 0.65 | 119625 | Activating signal cointegrator 1 | 119625 | | Hs.246112 | ASCC3L1 | 2q11.2 |
| | | | | | complex subunit 3-like 1 | | | | | |
| 28 | 0.776 | 1.469 | 0.528 | 27217 | Transcribed locus | 27217 | | Hs.410148 | | |
| 29 | 0.819 | 1.739 | 0.471 | 200260 | Cytochrome P450, family 3, | 200260 | R96774 | Hs.111944 | CYP3A7 | 7q21-q22.1 |
| | | | | | subfamily A, polypeptide 7 | | | | | |
| 30 | 0.761 | 1.423 | 0.535 | 32102 | Transcribed locus | 49443 | H15482 | Hs.22930 | | |
| 31 | 0.72 | 1.192 | 0.604 | 151231 | C-type lectin domain family 9, | 151231 | H02425 | Hs.531189 | UNQ9341 | 12p13.31- |
| | | | | | member A | | | | | p13.2 |
| 32 | 0.855 | 1.398 | 0.612 | 31893 | Family with sequence similarity | 31893 | R17865 | Hs.522172 | FAM11A | Xq28 |
| | | | | | 11, member A | | | | | |
| 33 | 1.718 | 2.542 | 0.676 | 683190 | Transcribed locus, weakly similar | 683190 | | Hs.536673 | | |
| | | | | | to XP_512541.1 PREDICTED: | | | | | |
| | | | | | similar to hypothetical protein | | | | | |
| | | | | | [Pan troglodytes] | | | | | |
| 34 | 0.844 | 1.149 | 0.735 | 199177 | Glucosidase, alpha; neutral C | 199177 | R95789 | Hs.143261 | CAPN3 | 15q15.1- |
| | | | | | | | | | | q21.1 |
| 35 | 1.611 | 2.552 | 0.631 | 71211 | | 71211 | | Data not | | |
| | | | | | | | | found | | |
| 36 | 1.11 | 1.519 | 0.731 | 67002 | | 67002 | T70366 | Data not | | |
| | | | | | | | | found | | |
| 37 | 0.862 | 1.314 | 0.656 | 39640 | Synovial sarcoma translocation, | 39640 | BM840962 | Hs.404263 | SS18 | 18q11.2 |
| | | | | | chromosome 18 | | | | | |
| 38 | 1.149 | 1.705 | 0.674 | 270849 | Jumonji, AT rich interactive | 270849 | N42634 | Hs.269059 | JARID2 | 6p24-p23 |
| | | | | | domain 2 | | | | | |
| 39 | 1.365 | 1.95 | 0.7 | 198559 | KIAA0853 | 198559 | R94802 | Hs.136102 | KIAA0853 | 13q14.13 |
| 40 | 1.077 | 1.521 | 0.708 | 195875 | Chromosome 18 open reading | 195875 | R92320 | Hs.149363 | C18orf1 | 18p11.2 |
| | | | | | frame 1 | | | | | |
| 41 | 1.104 | 1.651 | 0.669 | 119429 | Ring finger protein 10 | 119429 | T94904 | Hs.442798 | RNF10 | 12q24.31 |
| 42 | 1.304 | 1.803 | 0.723 | 253268 | SDS3 protein | 3573662 | BF431222 | Hs.416630 | SDS3 | 12q24.23 |
| 43 | 0.887 | 1.166 | 0.761 | 40300 | SYF2 homolog, RNA splicing | 5933194 | BQ052067 | Hs.20013 | P29 | 1p36.11 |
| | | | | | factor (S. cerevisiae) | | | | | |
| 44 | 0.658 | 1.011 | 0.651 | 26863 | TBC1 domain family, member 8 | 26863 | R13756 | Hs.442657 | TBC1D8 | 2q11.2 |
| | | | | | (with GRAM domain) | | | | | |
| 45 | 1.262 | 1.858 | 0.679 | 182864 | Uveal autoantigen with coiled-coil | 182864 | H45100 | Hs.33032 | UACA | 15q22-q24 |
| | | | | | domains and ankyrin repeats | | | | | |
| 46 | 0.803 | 1.098 | 0.731 | 41803 | Splicing factor, arginine/serine- | 41803 | R59287 | Hs.515271 | SFRS14 | 19p12 |
| | | | | | rich 14 | | | | | |
| 47 | 0.681 | 1.343 | 0.507 | 30262 | Coiled-coil domain containing 14 | 4560236 | BG323782 | Hs.17731 | FLJ12892 | 3q21.1 |
| 48 | 1.174 | 1.848 | 0.635 | 186852 | Ribosomal protein S6 kinase, | 186852 | R88127 | Hs.147119 | RPS6KA2 | 6q27 |
| | | | | | 90 kDa, polypeptide 2 | | | | | |
| 49 | 0.96 | 1.311 | 0.732 | 45921 | Hypothetical protein MGC11332 | 45921 | H08971 | Hs.98798 | MGC11332 | 2q12.1 |
| 50 | 1.002 | 1.466 | 0.683 | 29988 | | | | | | |
| 51 | 1.139 | 1.602 | 0.711 | 376888 | S100 calcium binding protein, | 376888 | | Hs.422181 | S100B | 21q22.3 |
| | | | | | beta (neural) | | | | | |
| 52 | 0.75 | 1.096 | 0.684 | 27687 | Transcribed locus | 27687 | R13012 | Hs.412408 | | |
| 53 | 1.05 | 1.52 | 0.691 | 250092 | Heterogeneous nuclear | 250092 | N23517 | Hs.808 | HNRPF | 10q11.21- |
| | | | | | ribonucleoprotein F | | | | | q11.22 |
| 54 | 0.957 | 1.579 | 0.606 | 123229 | Lines homolog 1 (Drosophila) | 123229 | R00425 | Hs.105633 | WINS1 | 15q26.3 |
| 55 | 0.523 | 0.991 | 0.528 | 27870 | Mesenchyme homeo box 2 | 27870 | R13205 | Hs.510504 | MEOX2 | 7p22.1-p21.3 |
| | | | | | (growth arrest-specific homeo | | | | | |
| | | | | | box) | | | | | |
| 56 | 0.73 | 1.166 | 0.626 | 42773 | Similar to kinase suppressor of | 42773 | R59802 | Hs.535123 | | 18p11.21 |
| | | | | | ras | | | | | |
| 57 | 0.884 | 1.48 | 0.597 | 28361 | Ras-related GTP binding D | 28361 | R13372 | Hs.485938 | RRAGD | 6q15-q16 |
| 58 | 0.85 | 1.204 | 0.706 | 39167 | Acyl-Coenzyme A oxidase 3, | 39167 | R54351 | Hs.479122 | ACOX3 | 4p15.3 |
| | | | | | pristanoyl | | | | | |
| 59 | 0.941 | 1.422 | 0.662 | 42439 | | 42439 | R61106 | Hs.20107 | | |
| 60 | 1.136 | 1.593 | 0.713 | 240937 | Peptidylprolyl isomerase domain | 240937 | H90997 | Hs.121432 | KIAA0073 | 5q12.3 |
| | | | | | and WD repeat containing 1 | | | | | |
| 61 | 0.901 | 1.375 | 0.655 | 50990 | Family with sequence similarity | 50990 | H18544 | Hs.499705 | FAM13C1 | 10q21.1 |
| | | | | | 13, member C1 | | | | | |
| 62 | 0.866 | 1.15 | 0.753 | 239881 | Transcribed locus | 239881 | H81996 | Hs.560895 | | |
| 63 | 1.538 | 2.449 | 0.628 | 252993 | CDNA FLJ46752 fis, clone | 252993 | H88581 | Hs.370024 | | |
| | | | | | TRACH3023063 | | | | | |
| 64 | 0.995 | 1.689 | 0.589 | 126592 | COBL-like 1 | 126592 | R06875 | Hs.470457 | COBLL1 | 2q24.3 |
| 65 | 1.145 | 1.505 | 0.761 | 239874 | Transcribed locus | 239874 | H79772 | Hs.40061 | | |
| 66 | 0.916 | 1.265 | 0.724 | 33196 | Similar to BcDNA: GH11415 gene | 33196 | R18950 | Hs.151443 | LOC151963 | 3q28-q29 |
| | | | | | product | | | | | |
| 67 | 0.862 | 1.579 | 0.546 | 192364 | Clone 24841 mRNA sequence | 192364 | H39125 | Hs.4892 | | |
| 68 | 1.186 | 1.769 | 0.67 | 186831 | Zinc finger protein 32 (KOX 30) | 186831 | R88121 | Hs.522885 | ZNF32 | 10q22-q25 |
| 69 | 1.351 | 1.837 | 0.735 | 195513 | Alanine-glyoxylate | 195513 | R91749 | Hs.34494 | AGXT2 | 5p13 |
| | | | | | aminotransferase 2 | | | | | |
| 70 | 0.847 | 1.551 | 0.546 | 32305 | Ets variant gene 1 | 39786 | R53267 | Hs.22634 | ETV1 | 7p22 |
| 71 | 1.29 | 2.029 | 0.636 | 346396 | Homeodomain interacting protein | 4933116 | BG820204 | Hs.397465 | HIPK2 | 7q32-q34 |
| | | | | | kinase 2 | | | | | |
| 72 | 0.869 | 1.209 | 0.719 | 51606 | Hypothetical LOC389182 | 51606 | H19377 | Hs.22347 | | 3q27.1 |
| 73 | 0.96 | 1.374 | 0.699 | 49906 | Transcribed locus | 49906 | H15234 | Hs.7847 | | |
| 74 | 0.697 | 1.04 | 0.67 | 347751 | Gardner-Rasheed feline sarcoma | 347751 | W81591 | Hs.1422 | FGR | 1p36.2-p36.1 |
| | | | | | viral (v-fgr) oncogene homolog | | | | | |
| 75 | 0.911 | 1.327 | 0.687 | 28147 | Megalencephalic | 28147 | Unresolved | Hs.517729 | MLC1 | 22q13.33 |
| | | | | | leukoencephalopathy with | | | | | |
| | | | | | subcortical cysts 1 | | | | | |
| 76 | 0.895 | 1.332 | 0.672 | 30103 | MRNA full length insert cDNA | 30103 | R16307 | Hs.21754 | | |
| | | | | | clone EUROIMAGE 30103 | | | | | |
| 77 | 1.201 | 2.009 | 0.598 | 665661 | Hypothetical protein MGC29671 | 665661 | | Hs.511912 | MGC29671 | 17p13.2 |
| 78 | 1.696 | 2.756 | 0.615 | 286320 | Eukaryotic translation initiation | 3432142 | BG055080 | Hs.539684 | EIF2S3 | Xp22.2- |
| | | | | | factor 2, subunit 3 gamma. 52 kDa | | | | | p22.1 |
| 79 | 0.691 | 1.105 | 0.625 | 26217 | CDC23 (cell division cycle 23, | 3918656 | BE892625 | Hs.153546 | CDC23 | 5q31 |
| | | | | | yeast, homolog) | | | | | |
| 80 | 0.979 | 1.284 | 0.762 | 35478 | Ubiquitin specific peptidase 43 | 35478 | R25687 | Hs.23935 | USP43 | 17p13.1 |
| 81 | 0.953 | 1.244 | 0.766 | 418296 | | 418296 | W90797 | Hs.26232 | | |
| 82 | 0.894 | 1.158 | 0.772 | 110747 | Chemokine-like factor | 5298687 | T83106 | Hs.15159 | CKLF | 16q22.1 |
| 83 | 1.302 | 1.704 | 0.764 | 198476 | STEAP family member 3 | 198476 | R94668 | Hs.20805 | TSAP6 | 2q14.2 |
| 84 | 0.858 | 1.268 | 0.677 | 33860 | Transcribed locus, moderately | 33860 | BM842766 | Hs.326475 | | |
| | | | | | similar to NP_008471.1 NADH | | | | | |
| | | | | | dehydrogenase subunit 1 [Canis | | | | | |
| | | | | | familiaris] | | | | | |
| 85 | 0.88 | 1.156 | 0.761 | 146567 | T-complex-associated-testis- | 146567 | R80870 | Hs.25094 | TCTE3 | 6q27 |
| | | | | | expressed 3 | | | | | |
| 86 | 0.96 | 1.376 | 0.698 | 38826 | Potassium voltage-gated | 39188 | R54540 | Hs.416139 | KCNA1 | 12p13 |
| | | | | | channel, shaker-related | | | | | |
| | | | | | subfamily, member 1 (episodic | | | | | |
| | | | | | ataxia with myokymia) | | | | | |
| 87 | 1.155 | 1.518 | 0.761 | 504180 | Hypothetical gene supported by | 504180 | AA132039 | Hs.399719 | | 6p22.3 |
| | | | | | AK026189 | | | | | |
| 88 | 0.895 | 1.215 | 0.737 | 50282 | Microtubule-associated protein | 50282 | H17600 | Hs.535786 | MAP1B | 5q13 |
| | | | | | 1B | | | | | |
| 89 | 1 | 1.498 | 0.668 | 109836 | GRIP and coiled-coil domain | 109836 | T85141 | Hs.521168 | GCC1 | 7q32.2 |
| | | | | | containing 1 | | | | | |
| 90 | 1.065 | 1.636 | 0.651 | 151943 | 5-azacytidine induced 2 | 151943 | H03342 | Hs.444724 | AZI2 | 3p24.1 |
| 91 | 1.254 | 1.864 | 0.673 | 182717 | Valyl-tRNA synthetase | 182717 | H43482 | Hs.520026 | VARS2 | 6p21.3 |
| 92 | 1.085 | 1.349 | 0.804 | 177057 | Hypothetical protein LOC149351 | 177057 | H40961 | Hs.546492 | LOC149351 | 1p22.2 |

TABLE 3
Primary Outcome Measures: Pre-Term Delivery Less than 34 Weeks of Gestation

| | Geom | Geom | | | | | | | | |
|---|---|---|---|---|---|---|---|---|---|---|
| | mean of | mean of | Fold | | | | | | | |
| | ratios in | ratios in | difference | | | | | | | |
| | class 1: | class 2: | of geom | Unique | | | | | | |
| | No | Yes | means | id | Description | Clone | GB acc | UG cluster | Gene symbol | Map Location |
| 1 | 1.758 | 3.851 | 0.457 | 202410 | CDNA FLJ40954 fis, | 202410 | H52618 | Hs.513659 | | |
| | | | | | clone UTERU2010525 | | | | | |
| 2 | 0.823 | 1.374 | 0.599 | 323823 | Zinc finger protein 364 | 323823 | W46196 | Hs.523550 | ZNF364 | 1q21.1 |
| 3 | 0.933 | 1.475 | 0.633 | 195917 | Family with sequence | 195917 | R92625 | Hs.512235 | ITPR2 | 12p11 |
| | | | | | similarity 20, member C | | | | | |
| 4 | 1.801 | 3.797 | 0.474 | 238966 | Tubulin, alpha 3 | 4891728 | BI196362 | Hs.524395 | TUBA3 | 12q12-12q14.3 |
| 5 | 1.169 | 2.096 | 0.558 | 182717 | Valyl-tRNA synthetase | 182717 | H43482 | Hs.520026 | VARS2 | 6p21.3 |
| 6 | 0.983 | 1.423 | 0.691 | 245016 | Receptor tyrosine | 245016 | N76287 | Hs.98255 | ROR2 | 9q22 |
| | | | | | kinase-like orphan | | | | | |
| | | | | | receptor 2 | | | | | |
| 7 | 1.654 | 2.426 | 0.682 | 167085 | Synaptotagmin XI | 167085 | R89738 | Hs.32984 | SYT11 | 1q21.2 |
| 8 | 0.813 | 1.357 | 0.599 | 488037 | Sushi-repeat- | 3882497 | BE787518 | Hs.15154 | SRPX | Xp21.1 |
| | | | | | containing protein, X- | | | | | |
| | | | | | linked | | | | | |
| 9 | 0.971 | 1.919 | 0.506 | 683190 | Transcribed locus, | 683190 | | Hs.536673 | | |
| | | | | | weakly similar to | | | | | |
| | | | | | XP_512541.1 | | | | | |
| | | | | | PREDICTED: similar | | | | | |
| | | | | | to hypothetical protein | | | | | |
| | | | | | [Pan troglodytes] | | | | | |
| 10 | 1.231 | 1.573 | 0.783 | 239240 | Testis specific A2 | 239240 | H71468 | Hs.145925 | TSGA2 | 21q22.3 |
| | | | | | homolog (mouse) | | | | | |
| 11 | 1.323 | 1.96 | 0.675 | 198559 | KIAA0853 | 198559 | R94802 | Hs.136102 | KIAA0853 | 13q14.13 |
| 12 | 0.768 | 1.141 | 0.673 | 415891 | Hermansky-Pudlak | 415891 | W86390 | Hs.474436 | HPS4 | 22cen-q12.3 |
| | | | | | syndrome 4 | | | | | |
| 13 | 0.636 | 1.068 | 0.596 | 26823 | ATG5 autophagy | 26823 | R14042 | Hs.486063 | APG5L | 6q21 |
| | | | | | related 5 homolog (S. cerevisiae) | | | | | |
| 14 | 1.077 | 1.917 | 0.562 | 683068 | Hypothetical protein | 683068 | | Hs.424589 | MGC52498 | 1p32.3 |
| | | | | | MGC52498 | | | | | |
| 15 | 0.967 | 1.458 | 0.663 | 47408 | GrpE-like 2, | 47408 | H10463 | Hs.511816 | GRPEL2 | 5q32 |
| | | | | | mitochondrial (E. coli) | | | | | |
| 16 | 0.894 | 1.356 | 0.659 | 42081 | Hypothetical protein | 42081 | R61019 | Hs.555958 | MGC5242 | 7q33 |
| | | | | | MGC5242 | | | | | |
| 17 | 0.672 | 1.075 | 0.625 | 502522 | WW domain | 502522 | AA156977 | Hs.533440 | WWP1 | 8q21 |
| | | | | | containing E3 ubiquitin | | | | | |
| | | | | | protein ligase 1 | | | | | |
| 18 | 1.231 | 2.114 | 0.582 | 346396 | Homeodomain | 4933116 | BG820204 | Hs.397465 | HIPK2 | 7q32-q34 |
| | | | | | interacting protein | | | | | |
| | | | | | kinase 2 | | | | | |
| 19 | 1.13 | 1.585 | 0.713 | 504180 | Hypothelical gene | 504180 | AA132039 | Hs.399719 | | 6p22.3 |
| | | | | | supported by | | | | | |
| | | | | | AK026189 | | | | | |

TABLE 4
Primary Outcome Measures: Pre-Term Delivery Less Than 37 Weeks of Gestation

| | Geom | Geom | | | | | | | | |
|---|---|---|---|---|---|---|---|---|---|---|
| | mean of | mean of | Fold | | | | | | | |
| | ratios in | ratios in | difference | | | | | | | |
| | class 1: | class 2: | of geom | | | | | | Gene | Map |
| | No | Yes | means | Unique id | Description | Clone | GB acc | UG cluster | symbol | Location |
| 1 | 0.746 | 0.474 | 1.574 | 503670 | Zinc finger protein 403 | 503670 | AV662024 | Hs.514116 | ZNF403 | 17q12 |
| 2 | 0.996 | 0.759 | 1.312 | 231472 | | 231472 | H92449 | Hs.520636 | | |
| 3 | 0.927 | 1.478 | 0.627 | 195917 | Family with sequence | 195917 | R92625 | Hs.512235 | ITPR2 | 12p11 |
| | | | | | similarity 20, member C | | | | | |
| 4 | 1.041 | 1.635 | 0.637 | 270849 | Jumonji, AT rich interactive | 270849 | N42634 | Hs.269059 | JARID2 | 6p24-p23 |
| | | | | | domain 2 | | | | | |
| 5 | 0.825 | 1.242 | 0.664 | 323823 | Zinc finger protein 364 | 323823 | W46196 | Hs.523550 | ZNF364 | 1q21.1 |
| 6 | 0.717 | 1.638 | 0.438 | 200260 | Cytochrome P450, family 3, | 200260 | R96774 | Hs.111944 | CYP3A7 | 7q21-q22.1 |
| | | | | | subfamily A, polypeptide 7 | | | | | |
| 7 | 0.645 | 1.109 | 0.582 | 42773 | Similar to kinase | 42773 | R59802 | Hs.535123 | | 18p11.21 |
| | | | | | suppressor of ras | | | | | |
| 8 | 0.789 | 1.066 | 0.74 | 47374 | | 47374 | H10692 | Hs.134521 | | |
| 9 | 1.619 | 2.331 | 0.695 | 167085 | Synaptotagmin XI | 167085 | R89738 | Hs.32984 | SYT11 | 1q21.2 |
| 10 | 0.952 | 1.28 | 0.744 | 245016 | Receptor tyrosine kinase- | 245016 | N76287 | Hs.98255 | ROR2 | 9q22 |
| | | | | | like orphan receptor 2 | | | | | |
| 11 | 0.839 | 1.259 | 0.666 | 376599 | CDC-like kinase 1 | 376599 | AA046192 | Hs.433732 | CLK1 | 2q33 |
| 12 | 0.888 | 1.285 | 0.691 | 42081 | Hypothetical protein | 42081 | R61019 | Hs.555958 | MGC5242 | 7q33 |
| | | | | | MGC5242 | | | | | |
| 13 | 0.647 | 1.059 | 0.611 | 26823 | ATG5 autophagy related 5 | 26823 | R14042 | Hs.486063 | APG5L | 6q21 |
| | | | | | homolog (S. cerevisiae) | | | | | |
| 14 | 0.63 | 1.195 | 0.527 | 29331 | TruB pseudouridine (psi) | 29331 | R12880 | Hs.21187 | TRUB1 | 10q25.3 |
| | | | | | synthase homolog 1 (E. coli) | | | | | |
| 15 | 1.238 | 1.725 | 0.718 | 253268 | SDS3 protein | 3573662 | BF431222 | Hs.416630 | SDS3 | 12q24.23 |
| 16 | 0.957 | 1.326 | 0.722 | 366848 | Transcription factor 7-like 2 | 366848 | AA029516 | Hs.501080 | TCF7L2 | 10q25.3 |
| | | | | | (T-cell specific, HMG-box) | | | | | |
| 17 | 1.074 | 1.717 | 0.626 | 238885 | | 238885 | | Data not found | | |
| 18 | 0.858 | 1.362 | 0.63 | 47396 | Family with sequence | 47396 | H10439 | Hs.500419 | FAM35A | 10q23.2 |
| | | | | | similarity 35, member A | | | | | |
| 19 | 1.813 | 2.911 | 0.623 | 509859 | Epithelial protein lost in | 509859 | | Hs.525419 | EPLIN | 12q13 |
| | | | | | neoplasm beta | | | | | |

TABLE 5
Predictors of Delivery - Pre-Term Delivery Less Than 14 Days From Clinical Presentation
Performance of the Compound Covariate Predictor Classifier: Accuracy 80%

| Class | Sensitivity | Specificity | PPV | NPV |
|---|---|---|---|---|
| No | 73.10% | 85.70% | 90.50% | 63.20% |
| Yes | 85.70% | 73.10% | 63.20% | 90.50% |

Composition of classifier:
Table - Sorted by t-value:

| | | | | Geom | Geom | | |
|---|---|---|---|---|---|---|---|
| | | | | mean of | mean of | | |
| | | | | ratios in | ratios in | | |
| | | Parametric p | % CV | class 1: | class 2: | | |
| | t-value | value | support | No | Yes | Unique id | Description |
| 1 | −4.13 | 0.000243 | 100 | 1.004 | 1.637 | 29947 | Zic family member 4 |
| 2 | −4.04 | 0.000358 | 100 | 0.899 | 1.445 | 37458 | Hydroxysteroid (17-beta) |
| | | | | | | | dehydrogenase 6 |
| 3 | −3.98 | 0.000422 | 100 | 1.703 | 2.762 | 193713 | Torsin A interacting protein 1 |
| 4 | −3.97 | 0.000512 | 100 | 0.981 | 1.608 | 195917 | Family with sequence similarity |
| | | | | | | | 20, member C |
| 5 | −3.69 | 0.001103 | 100 | 1.021 | 1.712 | 47408 | GrpE-like 2, mitochondrial (E. coli) |
| 6 | −3.51 | 0.001496 | 100 | 1.114 | 1.916 | 238885 | |
| 7 | −3.4 | 0.001853 | 100 | 0.855 | 1.398 | 31893 | Family with sequence similarity |
| | | | | | | | 11, member A |
| 8 | −3.2 | 0.003175 | 100 | 0.776 | 1.469 | 27217 | Transcribed locus |
| 9 | −2.9 | 0.007255 | 100 | 0.761 | 1.423 | 32102 | Transcribed locus |
| 10 | −2.67 | 0.012293 | 100 | 0.884 | 1.48 | 28361 | Ras-related GTP binding D |
| 11 | −2.62 | 0.014756 | 100 | 0.72 | 1.192 | 151231 | C-type lectin domain family 9, |
| | | | | | | | member A |
| 12 | −2.6 | 0.016239 | 100 | 0.682 | 1.404 | 29331 | TruB pseudouridine (psi) |
| | | | | | | | synthase homolog 1 (E. coli) |
| 13 | −2.46 | 0.019447 | 100 | 0.399 | 0.641 | 172069 | Rap guanine nucleotide |
| | | | | | | | exchange factor (GEF)-like 1 |
| 14 | −2.45 | 0.020769 | 100 | 0.523 | 0.991 | 27870 | [symbol] |
| | | | | | | | (growth arrest-specific homeo |
| | | | | | | | box) |
| 15 | −2.42 | 0.022742 | 100 | 0.957 | 1.579 | 123229 | Lines homolog 1(Drosophila) |
| 16 | −2.42 | 0.022545 | 100 | 0.862 | 1.579 | 192364 | Clone 24841 mRNA sequence |
| 17 | −2.38 | 0.023844 | 100 | 0.782 | 1.428 | 200882 | Sex comb on midleg-like 1 |
| | | | | | | | (Drosophila) |
| 18 | −2.16 | 0.042664 | 100 | 0.819 | 1.739 | 200260 | Cytochrome P450, family 3, |
| | | | | | | | subfamily A, polypeptide 7 |
| 19 | −2.14 | 0.040876 | 100 | 1.026 | 1.938 | 195843 | Hypothetical protein FLJ20366 |
| 20 | −2.08 | 0.04797 | 100 | 0.847 | 1.551 | 32305 | Ets variant gene 1 |
| 21 | −1.73 | 0.097283 | 100 | 0.742 | 1.33 | 41775 | TAR DNA binding protein |
| 22 | −1.7 | 0.097417 | 100 | 0.652 | 1.237 | 133863 | TYRO3 protein tyrosine kinase |
| 23 | −1.69 | 0.102136 | 100 | 0.19 | 0.404 | 125697 | Hypothetical protein FLJ10986 |
| 24 | 2.55 | 0.015372 | 100 | 0.696 | 0.429 | 51754 | Filamin A, alpha (actin binding |
| | | | | | | | protein 280) |
| 25 | 2.82 | 0.00812 | 100 | 0.464 | 0.288 | 213142 | Adenylate cyclase 3 |
| 26 | 2.84 | 0.008261 | 100 | 0.591 | 0.364 | 222563 | [symbol] |
| | | | | | | | (cDNA clone MGC: 28641 |
| | | | | | | | IMAGE: 4223847) |
| 27 | 2.95 | 0.005484 | 100 | 0.498 | 0.303 | 132341 | Keratin 7 |
| 28 | 2.99 | 0.005246 | 100 | 0.665 | 0.375 | 47178 | Chromosome 21 open reading |
| | | | | | | | frame 42 |
| 29 | 3.11 | 0.003875 | 100 | 0.691 | 0.4 | 151515 | Hypothetical protein MGC26963 |
| 30 | 3.18 | 0.00361 | 100 | 0.501 | 0.284 | 213747 | Myosin IB |
| 31 | 3.59 | 0.001056 | 100 | 0.541 | 0319 | 32221 | Rho GDP dissociation inhibitor |
| | | | | | | | (GDI) alpha |
| 32 | 3.67 | 0.000896 | 100 | 0.659 | 0.384 | 40134 | Major facilitator superfamily |
| | | | | | | | domain containing 3 |
| 33 | 3.94 | 0.000362 | 100 | 0.752 | 0.438 | 152863 | Phospholipid transfer protein |
| 34 | 3.94 | 0.000349 | 100 | 0.94 | 0.586 | 486465 | Transcribed locus |
| 35 | 4.22 | 0.000231 | 100 | 0.689 | 0.407 | 327676 | Keratin 13 |

| | | | | UG | Gene | Map |
|---|---|---|---|---|---|---|
| | Annotations | Clone | GB acc | cluster | symbol | Location |
| 1 | Info | 29947 | | Hs.415766 | ZIC4 | 3q24 |
| 2 | Info | 37458 | R35197 | Hs.524513 | RODH | 12q13 |
| 3 | Info | 193713 | H47769 | Hs.496459 | LAP1B | 1q24.2 |
| 4 | Info | 195917 | R92625 | Hs.512235 | ITPR2 | 12p11 |
| 5 | Info | 47408 | H10463 | Hs.511816 | GRPEL2 | 5q32 |
| 6 | Info | 238885 | | Data not | | |
| | | | | found | | |
| 7 | Info | 31893 | R17865 | Hs.522172 | FAM11A | Xq28 |
| 8 | Info | 27217 | | Hs.410148 | | |
| 9 | Info | 49443 | H15482 | Hs.22930 | | |
| 10 | Info | 28361 | R13372 | Hs.485938 | RRAGD | 6q15-q16 |
| 11 | Info | 151231 | H02425 | Hs.531189 | UNQ9341 | 12p13.31-p13.2 |
| 12 | Info | 29331 | R12880 | Hs.21187 | TRUB1 | 10q25.3 |
| 13 | Info | 172069 | H18802 | Hs.158530 | RAPGEFL1 | 17q21.1-q21.2 |
| 14 | Info | 27870 | R13205 | Hs.510504 | MEOX2 | 7p22.1-p21.3 |
| 15 | Info | 123229 | R00425 | Hs.105633 | WINS1 | 15q26.3 |
| 16 | Info | 192364 | H3g125 | Hs.4892 | | |
| 17 | Info | 200882 | R98881 | Hs.109655 | SCML1 | Xp22.2-p22.1 |
| 18 | Info | 200260 | R96774 | Hs.111944 | CYP3A7 | 7q21-q22.1 |
| 19 | Info | 195843 | R92305 | Hs.390738 | FLJ20366 | 8q23.2 |
| 20 | Info | 39786 | R53267 | Hs.22634 | ETV1 | 7p22 |
| 21 | Info | 41775 | Unresolved | Hs.300624 | TARDBP | 1p36.22 |
| 22 | Info | 133863 | R28238 | Hs.381282 | TYRO3 | 15q15.1-q21.1 |
| 23 | Info | 125697 | R07554 | Hs.444301 | FLJ10986 | 1p32.1 |
| 24 | Info | 51754 | N/A | Hs.195464 | FLNA | Xq28 |
| 25 | Info | 213142 | H69584 | Hs.546428 | MGC11266 | 2p23.3 |
| 26 | Info | 5702324 | BM994858 | Mm.182340 | Oxa1I | 14 C1 |
| 27 | Info | 132341 | R26301 | Hs.411501 | KRT7 | 12q12-q13 |
| 28 | Info | 3223091 | BE672389 | Hs.234016 | C21orf42 | 21q21.3 |
| 29 | Info | 151515 | H03751 | Hs.48343 | MGC26963 | 4q25 |
| 30 | Info | 213747 | H71756 | Hs.439620 | MYO1B | 2q12-q34 |
| 31 | Info | 5180471 | BI821973 | Hs.159161 | ARHGDIA | 17q25.3 |
| 32 | Info | 40134 | R53347 | Hs.7678 | LOC113655 | 8q24.3 |
| 33 | Info | 152863 | | Hs.439312 | PLTP | 20q12-q13.1 |
| 34 | Info | 486465 | BM686432 | Hs.553244 | | |
| 35 | Info | 327676 | W35201 | Hs.463032 | KRT13 | 17q12-q21.2 |

TABLE 6
Predictors of Delivery - Pre-Term Delivery Less Than 37 Weeks Gestation
Performance of the Support Vector Machine Classifier: Accuracy 88%

| Class | Sensitivity | Specificity | PPV | NPV |
|---|---|---|---|---|
| No | 78.90% | 95.20% | 93.80% | 83.30% |
| Yes | 95.20% | 78.90% | 83.30% | 93.80% |

Composition of classifier:
Table - Sorted by t-value:

| | | | | Geom | Geom | | |
|---|---|---|---|---|---|---|---|
| | | | | mean of | mean of | | |
| | | Parametric | % CV | ratios in | ratios in | | |
| | t-value | p-value | support | class 1: | class 2: | Unique id | Description |
| 1 | −3.87 | 0.000656 | 98 | 0.927 | 1.478 | 195917 | Family with sequence similarity |
| | | | | | | | 20, member C |
| 2 | −3.69 | 0.001054 | 98 | 1.041 | 1.635 | 270849 | Jumonji, AT rich interactive |
| | | | | | | | domain 2 |
| 3 | −3.28 | 0.002359 | 100 | 1.713 | 2.912 | 183120 | transcriptional activation, subunit |
| | | | | | | | 6, 77 kDa |
| 4 | −3.21 | 0.003554 | 100 | 0.645 | 1.109 | 42773 | Similar to kinase suppressor of |
| | | | | | | | ras |
| 5 | −3.13 | 0.003917 | 98 | 1.074 | 1.717 | 238885 | |
| 6 | −3.03 | 0.004785 | 95 | 0.879 | 1.377 | 30591 | monooxygenase/tryptophan 5- |
| | | | | | | | monooxygenase activation |
| 7 | −2.77 | 0.010683 | 100 | 0.647 | 1.059 | 26823 | ATG5 autophagy related 5 |
| | | | | | | | homolog (S. cerevisiae) |
| 8 | −2.69 | 0.012499 | 98 | 0.858 | 1.362 | 47396 | Family with sequence similarity |
| | | | | | | | 35, member A |
| 9 | −2.58 | 0.017524 | 100 | 0.717 | 1.638 | 200260 | Cytochrome P450, family 3, |
| | | | | | | | subfamily A, polypeptide 7 |
| 10 | −2.51 | 0.017381 | 82 | 0.627 | 0.946 | 34308 | Small nuclear RNA activating |
| | | | | | | | complex, polypeptide 3, 50 kDa |
| 11 | −2.49 | 0.019193 | 100 | 0.489 | 0.889 | 27870 | (growth arrest-specific homeo |
| | | | | | | | box) |
| 12 | −2.4 | 0.025071 | 100 | 0.63 | 1.195 | 29331 | TruB pseudouridine (psi) |
| | | | | | | | synthase homolog 1 (E. coli) |
| 13 | −2.34 | 0.027438 | 82 | 0.591 | 0.908 | 27210 | Catenin (cadherin-associated |
| | | | | | | | protein), alpha 2 |
| 14 | −2.34 | 0.027511 | 98 | 0.576 | 0.918 | 28140 | Dual specificity phosphatase 7 |
| 15 | −2.22 | 0.034106 | 100 | 0.748 | 1.272 | 200882 | Sex comb on midleg-like 1 |
| | | | | | | | (Drosophila) |
| 16 | −2.2 | 0.036317 | 50 | 0.822 | 1.399 | 192364 | Clone 24841 mRNA sequence |
| 17 | −2.15 | 0.041815 | 98 | 0.9 | 1.511 | 123706 | |
| 18 | −2.08 | 0.044956 | 98 | 0.784 | 1.22 | 66623 | Hepatocyte nuclear factor 4, |
| | | | | | | | alpha |
| 19 | −2.02 | 0.053696 | 98 | 0.892 | 1.543 | 183714 | WW domain binding protein 4 |
| | | | | | | | (formin binding protein 21) |
| 20 | −2.01 | 0.056232 | 98 | 0.774 | 1.252 | 505063 | Chromosome 21 open reading |
| | | | | | | | frame 108 |
| 21 | −1.98 | 0.058956 | 98 | 0.535 | 0.864 | 108220 | Early B-cell factor 2 |
| 22 | 2.21 | 0.033109 | 85 | 0.484 | 0.316 | 174543 | BAI1-associated protein 2 |
| 23 | 2.33 | 0.025475 | 100 | 0.406 | 0.239 | 113740 | Hypothetical protein FLJ20297 |
| 24 | 2.39 | 0.023539 | 100 | 1.131 | 0.528 | 124209 | WD repeat domain 37 |
| 25 | 2.46 | 0.018735 | 90 | 0.308 | 0.199 | 242007 | Uracil-DNA glycosylase |
| 26 | 2.62 | 0.012623 | 88 | 0.64 | 0.413 | 501718 | |
| 27 | 2.68 | 0.011144 | 100 | 0.22 | 0.132 | 502153 | Membrane-associated ring finger |
| | | | | | | | (C3HC4) 9 |
| 28 | 2.73 | 0.010533 | 92 | 0.464 | 0.297 | 162287 | Lipase, hormone-sensitive |
| 29 | 2.76 | 0.0103 | 90 | 0.727 | 0.471 | 145076 | Serpin peptidase Inhibitor, clade |
| | | | | | | | B (ovalbumin), member 6 |
| 30 | 2.89 | 0.006305 | 92 | 0.524 | 0.336 | 265267 | Heat shock 70 kDa protein 1A |
| 31 | 2.97 | 0.007276 | 98 | 0.746 | 0.474 | 503670 | Zinc finger protein 403 |
| 32 | 3.02 | 0.004464 | 100 | 0.256 | 0.136 | 341888 | Tripartite motif-containing 8 |
| 33 | 3.11 | 0.003511 | 75 | 0.991 | 0.656 | 265267 | Heat shock 70 kDa protein 1A |
| 34 | 3.51 | 0.001256 | 95 | 0.75 | 0.481 | 485088 | Transducin-like enhancer of split |
| | | | | | | | 2 (E(sp1) homolog, Drosophila) |
| 35 | 3.52 | 0.001148 | 78 | 0.994 | 0.654 | 486465 | Transcribed locus |
| 36 | 3.55 | 0.001101 | 100 | 0.366 | 0.212 | 501683 | Chromosome 20 open reading |
| | | | | | | | frame 24 |
| 37 | 3.65 | 0.000795 | 100 | 0.235 | 0.1 | 154581 | Zinc finger, HIT type 2 |
| 38 | 4.03 | 3.00E−04 | 100 | 1.483 | 0.922 | 160237 | Caveolin 2 |

| | | | | UG | Gene | Map |
|---|---|---|---|---|---|---|
| | Annotations | Clone | GB acc | cluster | symbol | Location |
| 1 | Info | 195917 | R92625 | Hs.512235 | ITPR2 | 12p11 |
| 2 | Info | 270849 | N42634 | Hs.269059 | JARID2 | 6p24-p23 |
| 3 | Info | 183120 | AI820821 | Hs.444931 | CRSP6 | 11q14 |
| 4 | Info | 42773 | R59802 | Hs.535123 | | 18p11.21 |
| 5 | Info | 238885 | | Data not | | |
| | | | | found | | |
| 6 | Info | 5740157 | BM557897 | Hs.74405 | YWHAQ | 2p25.1 |
| 7 | Info | 26823 | R14C42 | Hs.486063 | APG5L | 6q21 |
| 8 | Info | 47396 | H10439 | Hs.500419 | FAM35A | 10q23.2 |
| 9 | Info | 200260 | R96774 | Hs.111944 | CYP3A7 | 7q21-q22.1 |
| 10 | Info | 5634649 | BM730022 | Hs.546299 | SNAPC3 | 9p22.3 |
| 11 | Info | 27870 | R13205 | Hs.510504 | MEOX2 | 7p22.1-p21.3 |
| 12 | Info | 29331 | R12880 | Hs.21187 | TRUB1 | 10q25.3 |
| 13 | Info | 5262145 | BI553762 | Hs.167368 | CTNNA2 | 2p12-p11.1 |
| 14 | Info | 28140 | Unresolved | Hs.3843 | DUSP7 | 3p21 |
| 15 | Info | 200882 | R98881 | Hs.109655 | SCML1 | Xp22.2-p22.1 |
| 16 | Info | 192364 | H39125 | Hs.4892 | | |
| 17 | Info | 123706 | R02651 | Data not | | |
| | | | | found | | |
| 18 | Info | 66647 | T67216 | Hs.116462 | HNF4A | 20q12-q13.1 |
| 19 | Info | 183714 | H44535 | Hs.411300 | WBP4 | 13q14.11 |
| 20 | Info | 505063 | AA150920 | Hs.473611 | C21orf61 | 21q22.1 |
| 21 | Info | 108220 | T70765 | Hs.491292 | EBF2 | 8p21.2 |
| 22 | Info | 174543 | H22058 | Hs.128316 | BAIAP2 | 17q25 |
| 23 | Info | 5924479 | BQ062929 | Hs.516450 | FLJ20297 | 2q21.1 |
| 24 | Info | 124209 | R02417 | Hs.188495 | WDR37 | 10p15.3 |
| 25 | Info | 242007 | H94206 | Hs.191334 | UNG | 12q23-q24.1 |
| 26 | Info | 501718 | | Hs.458492 | | |
| 27 | Info | 502153 | AA126675 | Hs.65377 | MARCH- | 12q14.1 |
| | | | | | IX | |
| 28 | Info | 5744636 | H28089 | Hs.95351 | LIPE | 19q13.2 |
| 29 | Info | 145076 | R77378 | Hs.519523 | SERPIN86 | 6p25 |
| 30 | Info | 265267 | N27681 | Hs.520028 | HSPA1A | 6p21.3 |
| 31 | Info | 503670 | AV662024 | Hs.514116 | ZNF403 | 17q12 |
| 32 | Info | 341888 | | Hs.336810 | TRIM8 | 10q24.3 |
| 33 | Info | 265267 | N27681 | Hs.520028 | HSPA1A | 6p21.3 |
| 34 | Info | 485088 | AA039246 | Hs.332173 | TLE2 | 19p13.3 |
| 35 | Info | 486465 | BM686432 | Hs.553244 | | |
| 36 | Info | 501683 | Unresolved | Hs.184062 | C20orf24 | 20q11.23 |
| 37 | Info | 154581 | R55485 | Hs.121025 | ZNHIT2 | 11q13 |
| 38 | Info | 160237 | | Hs.212332 | CAV2 | 7q31.1 |

1. A method for detecting one or more pre-term labor marker polypeptide or pre-term labor polynucleotides in a subject comprising:
(a) obtaining a sample from a subject;
(b) detecting in polypeptides or polynucleotides extracted from the sample one or more pre-term labor polypeptide or pre-term labor polynucleotide that are associated with pre-term labor; and
(c) comparing the detected amount with an amount detected for a standard.

2. A method of detecting pre-term labor in a subject, the method comprising comparing:
(a) levels of one or more pre-term labor polypeptides or pre-term labor polynucleotides markers that are extracted from a sample from the subject; and
(b) normal levels of expression of the markers in a control sample, wherein a significant difference in levels of markers, relative to the corresponding normal levels, is indicative of pre-term labor.

3. A method as claimed in claim 1 comprising:
(a) contacting a biological sample obtained from a subject with one or more binding agent that specifically binds to pre-term labor polypeptide markers or parts thereof; and
(b) detecting in the sample amounts of polypeptides that bind to the binding agents, relative to a predetermined standard or cut-off value, and therefrom determining the presence or absence of pre-term labor in the subject.

4. A method as claimed in claim 3 wherein the binding agent is an antibody.

5. A method for screening a subject for pre-term labor comprising (a) obtaining a biological sample from a subject; (b) detecting in polypeptides extracted from the sample the amount of one or more pre-term labor polypeptide markers; and (c) comparing the amount of markers detected to a predetermined standard, where detection of a level of markers different than that of a standard is indicative of pre-term labor.

6. A method as claimed in claim 5 which further comprises detecting multiple pre-term labor polypeptide markers.

7. A method for determining the presence or absence of one or more pre-term labor marker in a subject comprising detecting one or more pre-term labor polynucleotide in a sample from the subject and relating the detected amount to the presence of pre-term labor.

8. A method as claimed in claim 7 wherein the polynucleotide detected is mRNA.

9. A method of claim 8 wherein the polynucleotide is detected by
(a) contacting the sample with oligonucleotides that hybridize to the polynucleotides; and
(b) detecting in the sample levels of nucleic acids that hybridize to the polynucleotides relative to a predetermined standard or cut-off value, and therefrom determining the presence or absence of pre-term labor in the subject.

10. A method as claimed in claim 9 wherein the mRNA is detected using an amplification reaction.

11. A method as claimed in claim 10 wherein the amplification reaction is a polymerase chain reaction employing oligonucleotide primers that hybridize to the polynucleotides, or complements of such polynucleotides.

12. A method as claimed in claim 9 wherein the mRNA is detected using a hybridization technique employing oligonucleotide probes that hybridize to the polynucleotides or complements thereof, wherein the mRNA is detected by (a) isolating mRNA from the sample and combining the mRNA with reagents to convert it to cDNA; (b) treating the converted cDNA with amplification reaction reagents and primers that hybridize to the polynucleotides, to produce amplification products; (d) analyzing the amplification products to detect an amount of mRNA encoding one or more markers; and (e) comparing the amount of mRNA to an amount detected against a panel of expected values for normal tissue derived using similar primers.

13. A method for diagnosing and monitoring pre-term labor in a subject comprising isolating polynucleotides in a sample from the subject; and detecting polynucleotides encoding pre-term labor polypeptide markers in the sample wherein the presence of higher or lower levels of polynucleotides encoding pre-term labor polypeptide markers in the sample compared to a standard or control is indicative of pre-term labor.

14. A method for monitoring the progression of pre-term labor in a subject, the method comprising: (a) detecting in a sample from the subject at a first time point, one or more pre-term labor polypeptide or polynucleotide markers; (b) repeating step (a) at a subsequent point in time; and (c) comparing levels detected in steps (a) and (b), and thereby monitoring the progression of pre-term labor.

15. A diagnostic composition comprising an agent that binds to a pre-term labor polypeptide marker or hybridizes to a polynucleotide encoding a pre-term labor polypeptide marker.

16. A method for assessing the potential efficacy of a test agent for preventing, inhibiting, or reducing pre-term labor in a subject, the method comprising comparing: (a) levels of one or more pre-term labor polypeptide or polynucleotide markers, in a first sample obtained from a subject and exposed to the test agent, and (b) levels of the markers in a second sample obtained from the subject, wherein the sample is not exposed to the test agent, wherein a significant difference in the levels of expression of the markers in the first sample, relative to the second sample, is an indication that the test agent is potentially efficacious for preventing, inhibiting or reducing pre-term labor in the subject.

17. A method of assessing the efficacy of a therapy for preventing, inhibiting, or reducing pre-term labor in a subject, the method comprising comparing: (a) levels of one or more pre-term labor polypeptide or polynucleotide markers in a first sample obtained from the subject; and (b) levels of the markers in a second sample obtained from the subject following therapy, wherein a significant difference in the levels of expression of the markers in the second sample, relative to the first sample, is an indication that the therapy is efficacious for preventing, inhibiting, or reducing pre-term labor in the subject.

18. A method of selecting an agent for preventing, inhibiting or reducing pre-term labor in a subject the method comprising (a) obtaining a sample containing one or more pre-term labor polypeptide or polynucleotides from the subject; (b) separately exposing aliquots of the sample in the presence of a plurality of test agents; (c) comparing levels of one or more pre-term labor polypeptide or polynucleotide markers in each of the aliquots; and (d) selecting one of the test agents which alters the levels of markers in the aliquot containing that test agent, relative to other test agents.

19. A method of preventing, inhibiting, or reducing pre-term labor in a subject, the method comprising (a) obtaining a sample containing one or more pre-term labor polypeptide or polynucleotides from the subject; (b) separately maintaining aliquots of the sample in the presence of a plurality of test agents; (c) comparing levels of one or more pre-term labor polypeptide or polynucleotide markers in each of the aliquots; and (d) administering to the subject at least one of the test agents which alters the levels of markers in the aliquot containing that test agent, relative to other test agents.

20. A method of assessing the potential of a test compound to cause pre-term labor, the method comprising: (a) maintaining separate aliquots of samples containing one or more pre-term labor polypeptide or polynucleotides in the presence and absence of the test compound; and (b) comparing expression of one or more pre-term labor polypeptide or polynucleotide markers, in each of the aliquots, and wherein a significant difference in levels of markers in the aliquot maintained in the presence of the test compound, relative to the aliquot maintained in the absence of the test compound, is an indication that the test compound potentially causes pre-term labor.

21. A method of claim 1 wherein the markers are one or more of the polynucleotides or polypeptides encoded by the polynucleotides in Table 1, 2, 3, 4, 5, and/or 6.

22. A method of claim 2 wherein the pre-term labour is less than 48 hours of clinical presentation and wherein the markers are one or more of the polynucleotides or polypeptides encoded by the polynucleotides in Table 1.

23. A method of claim 2 wherein the pre-term labour is less than 14 days of clinical presentation and wherein the markers are one or more of the polynucleotides or polypeptides encoded by the polynucleotides in Table 2.

24. A method of claim 2 wherein the pre-term labour is less than 34 weeks of gestation and wherein markers are one or more of the polynucleotides or polypeptides encoded by the polynucleotides in Table 3.

25. A method of claim 2 wherein the pre-term labour is less than 37 weeks of gestation and wherein the markers are one or more of the polynucleotides or polypeptides encoded by the polynucleotides in Table 4.

26. A method of claim 2 wherein the pre-term labour is less than 48 hours of clinical presentation and wherein the markers are one or more of the polynucleotides or polypeptides encoded by the polynucleotides in Table 5.

27. A method of claim 2 wherein less than 34 weeks of gestation the markers are one or more of the polynucleotides or polypeptides encoded by the polynucleotides in Table 6.

28. A method of claim 1 wherein the sample is maternal peripheral blood cells, more particularly mononuclear leukocytes.
